(19) United States
(12) Patent Application Publication
(10) Pub. No.: US 2025/0312585 A1
(43) Pub. Date: Oct. 9, 2025

(54) SYSTEMS AND METHODS FOR IMPROVED TRANSPORT AND DISTRIBUTION OF AGENTS IN AND CHARACTERIZATION OF BIOLOGICAL TISSUE

(71) Applicant: Board of Regents, The University of Texas System, Austin, TX (US)

(72) Inventor: Baohong YUAN, Arlington, TX (US)

(21) Appl. No.: 18/863,510

(22) PCT Filed: May 10, 2023
(86) PCT No.: PCT/US2023/066817

§ 371 Date: Nov. 6, 2024

Related U.S. Application Data
(60) Provisional application No. 63/340,171, filed on May 10, 2022.

(51) Int. Cl. A61M 37/00; A61B 5/00; A61K 49/22

(52) U.S. Cl. CPC A61M 37/0092; A61B 5/0071; A61K 49/22; A61K 2123/00

(57) ABSTRACT

In one aspect, methods of treating and/or imaging biological tissue or a biological compartment are described herein. In some embodiments, a method comprises disposing a population of agents, such as a therapeutic agent, imaging agent, theranostic agent, or other agent in the biological compartment. The method further comprises applying a focused ultrasound beam to a first target region of the biological compartment to compress at least a portion of the first target region, and subsequently removing the focused ultrasound beam from the first target region to end the compression or compressive force applied to the first target region. In some cases, the focused ultrasound beam has a duty cycle greater than 5% and/or a frequency of 1-30 MHz. In some instances, applying the ultrasound beam creates a first order ultrasound oscillation wave within the biological compartment having a pressure gradient in one or more dimensions of 0.1 to 10 MPa/mm.

### CROSS-REFERENCE TO RELATED APPLICATIONS

This application claims the priority benefit pursuant to 35 U.S.C. § 119 (e) to U.S. Provisional Patent Application No. 63/340,171 filed on May 10, 2022, the contents of which are herein incorporated by reference in their entirety.

### FIELD

The technology described herein generally relates to ultrasound systems and, more particularly, to systems and methods employing ultrasound to improve transport and distribution of agents in biological tissue, including for treatment and/or imaging applications. Systems and method described herein further generally relate to characterizing biological tissue.

### BACKGROUND

Currently, the transport and distribution of therapeutic or diagnostic agents (such as drugs, drug carriers, or imaging contrast agents) in a tumor or other tissues are uncontrollable. Extremely low mobility in tissue interstitial space is one of the major barriers to agent delivery. Two natural transport mechanisms in interstitial space are (1) diffusion due to concentration gradient and (2) convection via natural interstitial fluid flow. Both depend on natural and random motions. Diffusion is a slow process, especially for relatively large-sized agents (>10 nm). Convection is usually disrupted due to pathologic conditions, such as elevated interstitial fluid pressure (IFP) in tumors. This disruption may lead to a heterogeneous distribution of an agent and failure of treatment or diagnosis.

For example, in chemotherapy, drugs are usually distributed non-uniformly. Some areas may accumulate a much lower concentration of a drug than other areas. Drug concentration can often be unintentionally distributed in a tumor's peripheral areas at a higher concentration than in central areas. Thus, an insufficient drug dosage can occur in the central areas, leading to a high risk of tumor regrowth and metastases. Similar issues can also occur in other methods in which agents are locally injected into a tumor. Therefore, the ability to externally accelerate agent transport and control its distribution, including in a tumor or other biological compartment or tissue, is highly desirable.

### SUMMARY

Ultrasound-based methods can be used to increase tissue permeability and thus agent diffusivity in tissue (ultrasound-enhanced delivery). Described herein are methods of using a focused ultrasound beam to accelerate agent transport. This approach can be referred to as “squeezing interstitial fluid via transfer of ultrasound momentum” (SIF-TUM). In some embodiments of this approach, as described further herein, an ultrasound beam gently “squeezes” the tissue in a small focal volume from all the directions and generates a macroscopic streaming of interstitial fluid. Not intending to be bound by theory, and for purposes of aiding understanding and visualization, the SIF-TUM approach disclosed herein can be imagined as analogous to three-dimensionally compressing and relaxing a small internal portion of a water-filled sponge. This approach (SIF-TUM) provides a unique ability to externally accelerate agent transport and control its distribution three-dimensionally, as described further herein.

In one aspect, methods of treating and/or imaging biological tissue or a biological compartment are described herein. Also described are methods of inducing movement of an agent within interstitial fluid of biological tissue. In some embodiments, a method described herein comprises disposing a population of agents, such as a therapeutic agent, imaging agent, theranostic agent, or other agent, in the biological compartment or biological tissue. The method further comprises applying a focused ultrasound beam to a first target region of the biological compartment to compress at least a portion of the first target region, and subsequently removing the focused ultrasound beam from the first target region. Removing the beam can end the compression or compressive force applied to the first target region.

Moreover, in some embodiments, the focused ultrasound beam has a duty cycle greater than 5%. In some cases, the duty cycle is greater than 10%, greater than 20%, greater than 30%, greater than 40%, greater than 50%, greater than 60%, greater than 70%, greater than 80%, or greater than 90%. In some embodiments, the duty cycle is 20-100%, 20-95%, 20-90%, 20-80%, 20-70%, 30-100%, 30-100%, 30-95%, 30-90%, 30-80%, 30-70%, 40-100%, 40-95%, 40-90%, 40-80%, 40-70%, 50-100%, 50-95%, 50-90%, 50-80%, 50-70%, 60-100%, 60-95%, 60-90%, 60-80%, 70-100%, 70-99%, 70-95%, 70-90%, 80-100%, 80-99%, 80-95%, 80-90%, 90-100%, 90-99%, or 90-95%.

Moreover, the first target region can be a region of the biological compartment or tissue in which the agent has a relatively high concentration compared to other regions of the biological compartment. The first target region, for instance, can be an injection site of the population of agents. It is further to be understood that the biological compartment can be any biological compartment not inconsistent with the technical objectives of the present disclosure, such as a specific organ or tissue. In some instances, the biological compartment is soft tissue. The biological compartment can also be a tumor or cancer cells. Additionally, in some implementations, the biological compartment comprises solid matrix material and fluid material. The solid matrix material, in some embodiments, comprises cells or tissue. In some instances, the fluid material comprises interstitial fluid.

Further, in some cases, the focused ultrasound beam of a method described herein has a relatively small focal volume, such as an ultrasound focal volume of less than 10 mm³ or less than 1 mm³. In some cases, the ultrasound focal volume is 0.1-10 mm³, 0.1-0.9 mm³, 0.3-0.9 mm³, or 0.5-0.9 mm³. However, in other embodiments, the focal volume of the focused ultrasound beam of a method described herein is not particularly limited. In some cases, for example, the focused ultrasound beam has a focal volume of up to 10,000 mm³, up to 5,000 mm³, up to 1,000 mm³, or up to 500 mm³. Other sizes are also possible. Moreover, in some instances, the focal volume can be selected such that the increase in the biological compartment (or inside the focal volume) does not reach an upper limit of a pre-set value during the course of carrying out the method. In some cases, the focal volume of the focused ultrasound beam is based on a biologically relevant thermal dose (e.g., a thermal dose equivalent to a specific time period of heating at 43° C.).

Moreover, in some embodiments of a method described herein, applying the focused ultrasound beam to the first target region forces at least a portion of the population of agents (or fluid comprising the agents) out of the first target region (or out of the focal volume of the ultrasound beam). For example, in some cases, applying the focused ultrasound beam creates a pressure gradient, wherein the gradient comprises a higher pressure within the ultrasound focal volume or first target region, and a lower pressure outside of the ultrasound focal volume or first target region. It is to be understood that such a gradient can be measured in one, two, or three dimensions individually, or in two dimensions or in all three dimensions simultaneously. In some embodiments, for example, applying a focused ultrasound beam in accordance with a method described herein provides or creates a first order ultrasound oscillation wave having a pressure gradient (∇P₁) in one, two, or three dimensions (e.g., in the x-direction, in the y-direction, and/or in the z-direction) of 0.1 MPa/mm to 10 MPa/mm. As described further herein, such a focused ultrasound beam can be provided by appropriate selection of various parameters of the ultrasound beam (e.g., frequency, pressure, duty cycle, power, etc.). Moreover, as described further herein, creating such a pressure gradient can provide improved movement of agents within a biological compartment, including in a non-random, controlled, directional, and/or externally stimulated manner. In some cases, such a pressure gradient in one, two, or three dimensions (or in two dimensions or in all three dimensions simultaneously) can be provided using a relatively high frequency (such as 1-30 MHz or 5-30 MHz) and a relatively low pressure, which may be particularly preferred in some instances.

Further, in some embodiments, the focal volume of the focused ultrasound beam has a peak pressure of 1 kPa to 10 MPa or 10 kPa to 1 MPa. However, the peak pressure used in a method described herein is not necessarily limited, and other values are also possible.

As described further herein, a method according to the present disclosure can include repeated exposures to ultrasound, in the same or different regions. In some embodiments, for example, a method described herein further comprises applying the focused ultrasound beam to a second target region of the biological compartment to compress at least a portion of the second target region, and removing the focused ultrasound beam from the second target region, wherein the second target region is different than the first target region. For example, the second target region can be adjacent to the first target region in some cases.

In this manner, at least a portion of the agent that previously moved out of the first target region and into the second target region due to the first application of ultrasound can be further moved or directed to yet another location or region within the biological compartment. Moreover, this process can be repeated any desired number of times (e.g., up to 10,000 times, up to 5,000 times, up to 1000 times, up to 500 times, up to 100 times, or up to 50 times) to direct or guide agents to any desired number of discrete locations or regions within the biological compartment. Thus, in some embodiments, a method described herein further comprises applying the focused ultrasound beam to n additional target regions of the biological compartment to compress at least a portion of the n additional target regions; and removing the focused ultrasound beam from the n additional target regions, wherein n is an integer up to 10,000. Moreover, in some cases, the focused ultrasound beam is applied to the n additional target regions in sequence or in a desired pattern to obtain a pre-selected distribution of the agent within the biological compartment.

Methods described herein can also be safe, including when considering individual and cumulative ultrasound exposures. In some embodiments, for example, exposure of the biological compartment to the focused ultrasound beam is associated with a mechanical index (MI) of less than 1.9.

Any type of agent not inconsistent with the technical objectives of the present disclosure may be used in a method described herein. For example, in some cases, the population of agents used in a method described herein changes size (e.g., average size) when exposed to the focused ultrasound beam and/or when exposed to a temperature change. In some cases, the population of agents comprises a USF contrast agent. Additionally, in some embodiments, the population of agents has an average size, prior to applying the focused ultrasound beam (or after applying the focused ultrasound beam), that is smaller than a pore size of tissue extracellular matrix of the biological compartment. In some instances, the population of agents has an average size, prior to applying the focused ultrasound beam (or after applying the focused ultrasound beam), of less than 300 nm (or less than 200 nm, less than 100 nm, or less than 80 nm). In some embodiments, the population of agents has an average size between 10 nm and 40 nm before and/or after applying the focused ultrasound beam, that is, in the absence or presence of the applied ultrasound.

Further, in some implementations, applying the focused ultrasound beam to the first target region (or to a second or nth additional target region) increases the temperature of the first target region by less than 5° C. or by less than 3° C., or by between 1 and 5° C. Such a temperature change can provide one or more advantages over other methods, as described further herein.

A method described herein can include steps in addition to ultrasound steps. For example, in some cases, a method described herein further comprises imaging the population of agents in the biological compartment, such as using USF imaging or other fluorescence imaging. Additionally, in some instances (e.g., methods of treating disease or diseased tissue such as tumor tissue), a method described herein further comprises releasing a payload from the population of agents, such as by the application of ultrasound, electromagnetic radiation, a magnetic field, or other external stimulus. A payload can also be released due to degradation of the agent over time in vivo or in the biological compartment (e.g., within a tumor), or due to the presence of a stimulus (e.g., pH) within the biological compartment.

According to some further embodiments, an ultrasound system is provided. An ultrasound system can comprise one or more ultrasound sources, a control system, an optional fluorophore excitation source, and an optional image recording device.

According to some even further embodiments, a method of characterizing a biological tissue is provided. A method can comprise disposing a population of ultrasound-switchable fluorophores in a first region of the biological tissue, applying a focused ultrasound beam to the first region to switch at least one fluorophore of the population from an off state to an on state, applying a beam of electromagnetic radiation to the first region to excite at least one fluorophore of the population in the on state, removing the focused ultrasound beam from the first region, and detecting a dynamic ultrasound fluorescence (USF) signal emitted by the population of fluorophores during a recovery period after removal of the focused ultrasound beam from the first region.

Additional objects, advantages, and novel features, and various embodiments of the technology will be set forth in part in the description that follows, and in part will become apparent to those skilled in the art upon examination of the following, or can be learned by practice of the invention.

### BRIEF DESCRIPTION OF THE DRAWINGS

Aspects of the present disclosure can be better understood with reference to the following drawings. The components and/or features in the drawings are not necessarily to scale, with emphasis instead being placed upon clearly illustrating the principles of the disclosure. Aspects of the technology presented herein are described in detail below with reference to the accompanying drawing figures, wherein:

FIG. 1 schematically illustrates an example ultrasound-switchable fluorescence (USF) imaging setup, in accordance with some aspects of the technology described herein;

FIG. 2A illustrates an example heat map of pressure, in accordance with some aspects of the technology described herein;

FIG. 2B illustrates an example heat map of pressure, in accordance with some aspects of the technology described herein;

FIG. 2C illustrates an example vector field model, in accordance with some aspects of the technology described herein;

FIG. 2D illustrates an example vector field model, in accordance with some aspects of the technology described herein;

FIG. 2E illustrates an example amplitude distribution, in accordance with some aspects of the technology described herein;

FIG. 2F illustrates an example amplitude distribution, in accordance with some aspects of the technology described herein;

FIG. 3A illustrates an example vector field model, in accordance with some aspects of the technology described herein;

FIG. 3B illustrates a speed model, in accordance with some aspects of the technology described herein;

FIG. 3C illustrates an example heat map of speed, in accordance with some aspects of the technology described herein;

FIG. 3D illustrates an example heat map of speed, in accordance with some aspects of the technology described herein;

FIG. 4A illustrates an example temperature change plot, in accordance with some aspects of the technology described herein;

FIG. 4B illustrates an example heat map of temperature, in accordance with some aspects of the technology described herein;

FIG. 5A illustrates an example concentration distribution of agents, in accordance with some aspects of the technology described herein;

FIG. 5B illustrates an example concentration distribution of agents, in accordance with some aspects of the technology described herein;

FIG. 5C illustrates an example concentration distribution of agents, in accordance with some aspects of the technology described herein;

FIG. 5D illustrates an example concentration distribution of agents, in accordance with some aspects of the technology described herein;

FIG. 5E illustrates an example concentration distribution plot of agents, in accordance with some aspects of the technology described herein;

FIG. 5F illustrates an example concentration distribution plot of agents, in accordance with some aspects of the technology described herein;

FIG. 5G illustrates an example concentration plot of agents, in accordance with some aspects of the technology described herein;

FIG. 5H illustrates an example concentration plot, in with some aspects of the technology described herein;

FIG. 5I illustrates an example concentration plot of agents, in accordance with some aspects of the technology described herein;

FIG. 5J illustrates an example concentration plot of agents, in accordance with some aspects of the technology described herein;

FIG. 5K illustrates an example concentration plot of agents, in accordance with some aspects of the technology described herein;

FIG. 6A illustrates an example heat map of a concentration distribution of agents, in accordance with some aspects of the technology described herein;

FIG. 6B illustrates an example heat map of a concentration distribution of agents, in accordance with some aspects of the technology described herein;

FIG. 6C illustrates an example heat map of a concentration distribution of agents, in accordance with some aspects of the technology described herein;

FIG. 7A illustrates an example concentration distribution of agents, in accordance with some aspects of the technology described herein;

FIG. 7B illustrates an example concentration distribution of agents, in accordance with some aspects of the technology described herein;

FIG. 7C illustrates an example concentration distribution of agents, in accordance with some aspects of the technology described herein;

FIG. 7D illustrates an example concentration distribution of agents, in accordance with some aspects of the technology described herein;

FIG. 7E illustrates an example concentration distribution plot of agents, in accordance with some aspects of the technology described herein;

FIG. 7F illustrates an example concentration distribution plot of agents, in accordance with some aspects of the technology described herein;

FIG. 7G illustrates an example concentration plot of agents, in accordance with some aspects of the technology described herein;

FIG. 7H illustrates an example concentration plot of agents, in accordance with some aspects of the technology described herein;

FIG. 7I illustrates an example concentration plot of agents, in accordance with some aspects of the technology described herein;

FIG. 7J illustrates an example concentration plot of agents, in accordance with some aspects of the technology described herein;

FIG. 8A illustrates an example temperature plot of an exposure, in accordance with some aspects of the technology described herein;

FIG. 8B is an example plot illustrating a relationship between concentration of an agent, pressure, and time, in accordance with some aspects of the technology described herein;

FIG. 8C is an example plot illustrating a relationship between concentration of an agent, pressure, and time, in accordance with some aspects of the technology described herein;

FIG. 9A is a CT image illustrating an example distribution of nanoparticles, in accordance with some aspects of the technology described herein;

FIG. 9B is a fluorescence image illustrating an example distribution of nanoparticles, in accordance with some aspects of the technology described herein;

FIG. 10A illustrates an example USF signal plot, in accordance with some aspects of the technology described herein;

FIG. 10B illustrates an example USF signal plot, in accordance with some aspects of the technology described herein;

FIG. 11A illustrates an example concentration plot, in accordance with some aspects of the technology described herein;

FIG. 11B graphically illustrates components of an example pressure gradient, in accordance with some aspects of the technology described herein;

FIG. 11C illustrates an example plot of a temperature increase as a function of time, in accordance with some aspects of the technology described herein;

FIG. 11D is an example plot illustrating a relationship between concentration, focal size, and exposure time, in accordance with some aspects of the technology described herein;

FIG. 12A illustrates a view of an example USF signal of a tumor, in accordance with some aspects of the technology described herein;

FIG. 12B illustrates a view of an example USF signal of a tumor, in accordance with some aspects of the technology described herein;

FIG. 13A is an example fluorescence image of a tumor, in accordance with some aspects of the technology described herein;

FIG. 13B is an example fluorescence image of a tumor, in accordance with some aspects of the technology described herein;

FIG. 13C is an example fluorescence image of a tumor, in accordance with some aspects of the technology described herein;

FIG. 13D is an example fluorescence image of a tumor, in accordance with some aspects of the technology described herein;

FIG. 14A illustrates an example USF-based squeezing interstitial fluid via transfer of ultrasound momentum (SIF-TUM) system, in accordance with some aspects of the technology described herein;

FIG. 14B illustrates an example USF-based squeezing interstitial fluid via transfer of ultrasound momentum (SIF-TUM) system, in accordance with some aspects of the technology described herein;

FIG. 15A illustrates example focusing arrangements for USF imaging and SIF-TUM imaging, in accordance with some aspects of the technology described herein;

FIG. 15B illustrates example imaging locations in relation to agent concentration, in accordance with some aspects of the technology described herein;

FIG. 15C illustrates an example method, in accordance with some aspects of the technology described herein;

FIG. 16A illustrates an example plot of agent concentration, in accordance with some aspects of the technology described herein;

FIG. 16B illustrates an example plot of exposure time at different or varying frequencies, in accordance with some aspects of the technology described herein;

FIG. 16C is an example plot illustrating a relationship between concentration, ultrasound frequency, and exposure time, in accordance with some aspects of the technology described herein;

FIG. 17A is a graphic describing the cellularity of example tumor tissue before a treatment, in accordance with some aspects of the technology described herein;

FIG. 17B is a graphic describing the cellularity of example tumor tissue after a treatment, in accordance with some aspects of the technology described herein;

FIG. 17C illustrates the cellularity of example tumor tissue before a treatment, in accordance with some aspects of the technology described herein;

FIG. 17D illustrates the cellularity of example tumor tissue after a treatment, in accordance with some aspects of the technology described herein;

FIG. 18A illustrates white light, planar fluorescence, and USF images of agents in example tissue, in accordance with some aspects of the technology described herein;

FIG. 18B illustrates a planar fluorescence image of agents in example tissue, in accordance with some aspects of the technology described herein;

FIG. 18C shows USF images illustrating a heterogeneous distribution of USF agents in example tissue, in accordance with some aspects of the technology described herein;

FIG. 18D shows image slices illustrating a distribution of USF agents as a function of depth, in accordance with some aspects of the technology described herein;

FIG. 19A is an image of an example USF signal, in accordance with some aspects of the technology described herein;

FIG. 19B is an image of an example USF signal, in accordance with some aspects of the technology described herein;

FIG. 20 is a plot of an example fluorescence signal as a function of time after an ultrasound is off, in accordance with some aspects of the technology described herein;

FIG. 21A illustrates an example spatial relationship between focal areas of SIF-TUM and USF exposures, in accordance with some aspects of the technology described herein;

FIG. 21B illustrates an example method, in accordance with some aspects of the technology described herein; and

FIG. 22 illustrates an example treatment cycle and monitoring plan, in accordance with some aspects of the technology described herein.

### DETAILED DECRIPTION

Embodiments described herein can be understood more readily by reference to the following detailed description, examples, and claims. Elements, apparatus and methods described herein, however, are not limited to the specific embodiments presented in the detailed description, examples, and claims. It should be recognized that these embodiments are merely illustrative of the principles of the present invention. Numerous modifications and adaptations will be readily apparent to those of skill in the art without departing from the spirit and scope of the invention.

Further, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

At a high level, described herein are high-resolution deep-tissue optical imaging systems and methods that in some instances utilize ultrasound-switchable fluorescence (USF) imaging. As will be appreciated, the use of USF imaging can overcome the poor spatial resolution (approximately 5 mm) of conventional fluorescence imaging in centimeters-deep tissue. USF has achieved a sub-millimeter resolution in centimeters-deep tissue. High resolution of USF can be achieved in some cases by using a tightly focused ultrasound beam and a temperature supersensitive and fluorophore-encapsulated nanoagent (or other USF contrast agent). In some such cases, the process comprises: (1) the focused ultrasound beam induces a slight temperature increase (e.g., a few degrees Celsius) in its small focal volume; (2) this increased temperature leads to a phase transition of the contrast agent and a significant reduction in size; (3) the encapsulated fluorophores experience a switch from a polar to a less polar nonpolar microenvironment; (4) this microenvironment change switches the environment-sensitive fluorophores on, and leads to a significant increase in fluorescence emission, which is referred to as USF signal and only comes from the small focal volume; and (5) by scanning the ultrasound focus and detecting the USF signal at each location, a high-resolution USF image can be acquired to indicate the distribution of the USF contrast agent. Thus, a USF image can indicate contrast agent distribution via fluorescence contrast in centimeters-deep tissues with high spatial resolution using nonionizing radiation and nonradioactive materials.

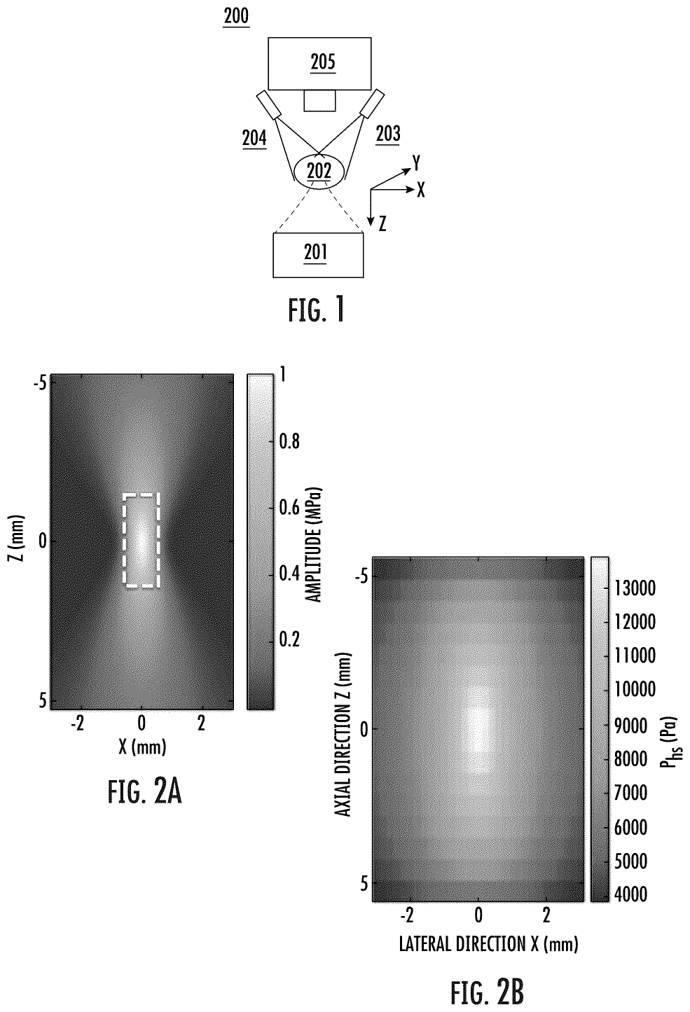

Briefly referring to FIG. 1 an example setup for use in USF imaging is illustrated. A tightly focused ultrasound transducer (FUST) can be used to deliver an ultrasound wave into a tumor (T). Two optical fiber bundles (LF) can used to deliver excitation light to illuminate the tumor, and a camera (EMCCD) can be used to detect the fluorescence emission from the USF contrast agents.

According to some aspects of the present technology, ultrasound is used to improve the movement and distribution of agents within biological tissue. As described herein, the presently disclosed SIF-TUM systems and methods can further be modeled via several equations. A simulation can be conducted to further describe this method and quantify the motions of agents under ultrasound illumination and their recovery after ultrasound exposure. The effects of various experimental parameters on the motions and transport efficiencies of agents are also described further below. As will be appreciated, SIF-TUM can provide a powerful approach for accelerating agent transport in deep tissue and controlling the distribution of the agent. Additionally, SIF-TUM can, in some cases, be combined with USF imaging.

According to some embodiments of the present technology a method of selectively transporting an agent within a porous material is provided. In some instances, the method can include disposing population of agents (e.g. such as a temperature-sensitive agent and/or a temperature insensitive agent), applying a focused ultrasound beam to a first target region of the porous material to compress at least a portion of the first target region, and removing the focused ultrasound beam from the first target region. In some instances, the focused ultrasound beam has a duty cycle greater than 5%, greater than 10%, greater than 15%, or greater than 20%. In applying the focused ultrasound beam, a first order ultrasound oscillation wave can be created that has a pressure gradient (∇P₁) in one, two, or three dimensions of 0.1 MPa/mm to 10 MPa/mm. Further, the focused ultrasound beam can have one or more other properties, for instance the focal volume of the focused ultrasound beam can have a peak pressure of about 1 kPa to about 10 MPa and/or the focused ultrasound beam can have a frequency of about 1 MHz to about 30 MHz.

According to some aspects of selectively transporting an agent within a porous material, a population of agents is disposed in the porous material and a focused ultrasound beam is applied to a target region of the porous material to compress at least a portion of the target region. In some aspects the target region can be a first target region, a second target region, and/or a nth additional target region. In some instances, the porous material comprises a solid matrix material, for example in some instances cells and/or tissue (e.g. biological tissue), and a fluid material, for example in some instances interstitial fluid. Further, the porous material can include one or more biological compartments. In some other instances, the porous material can be a non-biological material such as an inorganic material (e.g., a porous ceramic material) or a non-biological organic material such as a polymeric material (e.g., a polyurethane foam or other polymeric material). In some further instances, a porous material or porous media can be fiber or cellulose based material, a hydrogel based material, a zeolite, and/or a silica based material. Additionally, any combinations of these materials are also contemplated. In some instances, a porous material can have a pore size from about 1 μm to about 10 μm. In some other instances, a porous material can have a pore size of 2 μm to about 20 μm, a pore size of about 20 μm to about 1500 μm, a pore size of about 40 μm to about 200 μm, and/or a pore size of about 50 μm to about 200 μm.

When applying the focused ultrasound beam to a target region of the porous material (e.g. a first target region) the beam can force at least a portion of the population of agents out of the target region (i.e. out of the first target region). The method can further comprise applying the focused ultrasound beam to a second target region of the porous material to compress at least a portion of the second target region, and removing the focused ultrasound beam from the second target region. In some instances, the second target region is different than the first target region. In some other instances, the second target region is the same as the first target region. Additionally, the method can further comprise applying the focused ultrasound beam to n additional target regions of the porous material to compress at least a portion of the n additional target regions, and removing the focused ultrasound beam from the n additional target regions. In some instances, n can be an integer up to 10,000, up to 5,000, up to 1000, up to 500, up to 100, or up to 50. It is further to be understood that n can be any desired integer. As the focused ultrasound beam is applied to the target region of the porous material, the exposure of the porous material to the focused ultrasound beam can be associated or correspond with a mechanical index (MI) less than 1.9. In some instances, the focused ultrasound beam is applied using an ultrasound transducer array.

Looking more particularly at the agents and the activity thereof. In some instances, an agent or population of agents can comprise a USF contrast agent or another nanoagent. Further, an agent (or any number of agents in the population of agents) can change size when exposed to the focused ultrasound beam and/or when exposed to a temperature change. As will be appreciated, according to some aspects, an agent or the population of agents can have an average size that is smaller than a pore size of the matrix material prior to applying the focused ultrasound beam. In some other embodiments, the porous material can comprise a biological compartment and an agent or the population of agents can have an average size that is smaller than a pore size of a tissue extracellular matrix of a biological compartment of the porous material. In some instances, an agent or a population of agents has an average size of less than 300 nm prior to applying the focused ultrasound beam, and in some other instances, an agent or a population of agents has an average size of between about 10 nm and about 40 nm. According to some aspects, an agent or population of agents can be imaged. Further, in some a method further comprises releasing a payload from the population of agents, such as by the application of ultrasound, electromagnetic radiation, a magnetic field, or other external stimulus. A payload can also be released due to degradation of the agent over time in vivo or in the biological compartment (e.g., within a tumor), or due to the presence of a stimulus (e.g., pH) within the biological compartment.

The method of transporting an agent within a porous material can further comprise applying a focused ultrasound beam to a target region (e.g. first target region, second target region, nth target region) and can increase the temperature of that target region by less than 10° C., more specifically less than 5° C.

According to some other embodiments of the technology, an ultrasound system is provided, for example for use in a method of selectively transporting an agent within a porous material and/or a method of characterizing biological tissue. In some aspects an ultrasound system can include one or more ultrasound sources, a control system, a fluorophore excitation device and an image recording device.

According to some even further embodiments of the present technology, a method of characterizing a biological tissue is provided. In some aspects a method can be directed to cellularity changes in biological tissue. In some aspects a method comprises disposing a population of ultrasound-switchable fluorophores in a first region of the biological tissue, applying a focused ultrasound beam to the first region to switch at least one fluorophore of the population from an off state to an on state, applying a beam of electromagnetic radiation to the first region to excite at least one fluorophore of the population in the on state, removing the focused ultrasound beam from the first region, and detecting a dynamic ultrasound fluorescence (USF) signal emitted by the population of fluorophores during a recovery period after removal of the focused ultrasound beam from the first region. Additionally, a method can in some instances incorporate measuring or determining one or more additional metrics, for instance at least one of a recovery time constant (τ) for the first region and/or a transportability coefficient (M) for the first region. Subsequently, a method can further include disposing a population of ultrasound-switchable fluorophores in a second region of the biological tissue, applying a focused ultrasound beam to the second region to switch at least one fluorophore of the population from an off state to an on state, applying a beam of electromagnetic radiation to the second region to excite at least one fluorophore of the population in the on state, removing the focused ultrasound beam from the second region, and detecting a dynamic ultrasound fluorescence (USF) signal emitted by the population of fluorophores during a recovery period after removal of the focused ultrasound beam from the second region. As will be appreciated, in some instances the first region and the second region are different spatial regions of the biological tissue, in some other instances, the first region and the second region are the same spatial regions, and/or are overlapping spatial regions. In some instances, based on the applying the focused ultrasound beam and the beam of electromagnetic radiation, removing one or more of the beams, and the detected USF signal, the method can further include measuring or determining a recovery time constant (τ) for the second region and/or a transportability coefficient (M) for the second region.

In some further instances, a method can further subsequently include disposing a population of ultrasound-switchable fluorophores in each of n additional regions of the biological tissue (with n being, for example an integer from 1 to 10,000), applying a focused ultrasound beam to each of the n additional regions to switch at least one fluorophore of the population from an off state to an on state, applying a beam of electromagnetic radiation to each of the n additional regions to excite at least one fluorophore of the population in the on state, removing the focused ultrasound beam from each of the n additional regions, and detecting a dynamic ultrasound fluorescence (USF) signal emitted by the population of fluorophores during a recovery period after removal of the focused ultrasound beam from each of the n additional regions. In some instances, the n additional regions are each different spatial regions of the biological tissue than each other. In some other instances, the n additional regions are each different spatial regions of the biological tissue than the first region and the second region. Based at least on the detected USF signal during a recovery period, the method can further include measuring or determining a recovery time constant (τ) and/or a transportability coefficient (M) for the n additional regions, for example as an ultrasound beam is raster scanned.

Based on one or more measured or determined recovery time constant(s) (t) and/or transportability coefficient(s) (M), the recovery time constants and/or transportability coefficients for the first region, second region, and/or n additional regions can be averaged to provide, respectively, an average recovery time constant or an average transportability coefficient for the biological tissue at a clinical time point (e.g. at a first clinical time point, second clinical time point, etc.). Further, the average recovery time constant and/or the average transportability coefficient for the biological tissue can be correlated with an average characteristic of the biological tissue at the clinical time point (e.g. the first clinical time point, the second clinical time point, etc.). In some aspects a first clinical time point can be understood to be a given time point, such as a time point that is clinically relevant. For example a first clinical time point could be before chemotherapy, radiation therapy, or other therapy is applied to the biological tissue, which in some instances may be a tumor.

Accordingly, the recovery time constant can be measured at a first region, and/or a second region, and/or each of the n additional regions and the average characteristic of the biological tissue or material based on the correlating can correspond to an average cellularity of the biological tissue. As will be appreciated, cellularity can be understood to be or correspond to a cell density or number of cells per unit volume of tissue. Additionally, a change in cellularity can be understood as an increase or decrease in cell density or number of cells per unit volume of tissue as a function of time, such that a decrease in cellularity corresponds to cell death over time. As such, in some aspects the biological tissue can be a diseased biological tissue and a clinical time point (e.g. the first clinical time point) is a time point before a treatment is applied to the diseased biological tissue.

Accordingly, a method can further comprise measuring an average recovery time constant for the biological tissue at a second clinical time point and correlating the average recovery time constant for the biological tissue with an average cellularity of the biological tissue at the second clinical time point. As will be appreciated the first clinical time point can be different than the second clinical time point, and further the second clinical time point can be a time point after a treatment is applied to the diseased biological tissue. As will be appreciated, an average metric (e.g. recovery time constant and/or transportability coefficient) at the second clinical time point can be determined in the same or an analogous manner as for the first clinical time point. That is, the steps of the relevant method for measuring a metric can be repeated at a later time point (e.g. after treatment).

In some aspects, methods can further include comparing the average recovery time constant and/or average cellularity at the first clinical time point with the average recovery time constant and/or average cellularity at the second clinical time point, thereby identifying a change in cellularity of the biological tissue from the first clinical time point to the second clinical time point. Referring back to determining a recovery time constant (τ) for a first region and/or a second region and/or a nth region, the recovery time constant can in some instances be measured in accordance with Equation (1): y=−A*exp(−t/τ)+y₀; where y is the dynamic USF signal detected in the region (e.g. first region) during the recovery period, t is the recovery period in the region (e.g. first region), A is a constant of the region (e.g. first region), and y₀ is a constant of the region (e.g. first region). In some aspects, A and y₀ will be different in each region. As will be appreciated, Eq. (1) is one possible pathway to measuring or determining a time constant metric, and other equations such as multiple exponential term functions and decay functions may be used. However, as will be appreciated, generally the same determination function should be used for each region.

Continuing, in some further aspects, the method can include determining and/or measuring the transportability coefficient (M). The transportability coefficient (M) can be measured at the first region and each of the n additional regions and the average characteristic of the biological tissue is, or corresponds to, an average transportability of the biological tissue. In some aspects, transportability of a biological tissue can generally be understood as a metric intrinsic to the biological tissue that can quantify how readily an agent (such as a USF fluorophore or another agent disposed within biological tissue or interstitial fluid of biological tissue) moves within the biological tissue, such as by diffusion or some other action applicable to movement of a chemical and/or biological species within a tissue. In some instances, the transportability coefficient (M) is measured in accordance with Equation (2): M=R_{f}(k/μ)H, wherein k is tissue permeability, μ is tissue fluid viscosity, R_{f} is a retardation factor between interstitial fluid and the agent, and His the tissue apparent modulus.

The various embodiments of the present technology will now be discussed in more particular detail with regards to the following non-limiting examples. Further, various portions of the examples and the foregoing discussion of the technology methods that can be carried out. In some instances, methods include steps and/or blocks however these do not necessarily have to be carried out in a prescribed order and can further include additional steps and/or blocks or substeps. In some instances, a method does not necessarily have to require a given step.

### EXAMPLE 1

### Modelling of US-Induced Streaming of Interstitial Fluid

### A. General

The following Example describes the modelling of ultrasound-induced streaming of interstitial fluid in a millimeter focal zone in deep tissue. The interaction between ultrasound and tissue was simulated by considering both motions of solid and fluid in tissue. SIF-TUM gently squeezes the tissue in a small ultrasound focal volume from all directions and to generate a macroscopic streaming of interstitial fluid in a millimeter focal size. SIF-TUM provides a unique opportunity to externally accelerate agent transport and to control its distribution three-dimensionally in deep tissue. The success of this technology will contribute to the delivery efficiency and the related therapeutic and/or diagnostic efficiency of delivered or administered agents.

### B. Introduction

In some modeling, tissue can be treated as a single-phase material (e.g., either a pure fluid or solid, or a single-phase mixture), and the relative motion between the fluid and solid can be ignored. However, most biological soft tissues include both interstitial fluid and solid matrix, and their relative motion can occur when tissue interacts with an externally applied force or radiation. One visualization that can help illustrate this motion is the following. A groove can be observed on the skin after being pressed by a sharp object or long-term use of a rubber band. The mechanism of this groove formation, not intending to be bound by theory, is that the tissue fluid is pushed away from the applied area, and the solid matrix of tissue is deformed under the pressure. After completing the pressing, the skin will gradually recover (i.e., the groove will disappear), which typically takes a much longer time than the pressing time. This is because the fluid recovery speed only depends on the tissue properties during the recovery period, whereas the time to generate the deformation depends on both tissue property and the pressure property.

Therefore, when modeling SIF-TUM, both fluid and solid matrix and their relative motion are considered in the present disclosure. SIF-TUM gently squeezes the tissue in a small ultrasound focal volume from all directions (e.g., approximately 0.7 mm³ in this Example). Based on Darcy's law, the interstitial fluid can move relative to the solid matrix depending upon the hydrostatic pressure gradient. Conversely, when the fluid moves away from the original location, the space should be occupied by the solid matrix if no void space exists (i.e., no cavitation), which indicates the solid matrix is deformed. Therefore, SIF-TUM-induced fluid motion and solid matrix deformation can be differentiated from acoustic radiation force (ARF)-induced tissue displacement. The former can be described as a type of compression and expansion motion, whereas ARF-induced tissue displacement can be considered a translational motion, typically along a primary direction that is usually the force direction. In practice, these two motions may be superposed. One can imagine these two phenomena as follows (again not intending to be bound by theory). When an ultrasound beam is focused in tissue, in the focal volume, the ARF will push the tissue forward along the wave axial direction (z) and generate a small tissue displacement. Meanwhile, the ultrasound wave will also pass a portion of its momentum to the fluid, which will lead to a “splash” of the fluid moving out of the focal volume and thus the deformation of the solid matrix. Because the tissue hydraulic conductivity is usually small, it can function as a resistance to the motion of the fluid. Thus, the “splashed” fluid will be accumulated somewhere around the area, and it further leads to the elevation of the hydrostatic pressure in the focal volume. Mathematically, the spatial nonuniform momentum transfer at different locations in the focal volume will induce a nonuniform increase of fluid hydrostatic pressure (see Equation 1 in Table 1). Based on Darcy's law, the gradient of this hydrostatic pressure will drive the motion of the fluid, which is called an exudation flow, and will create a type of macroscopic streaming at a size slightly larger than the ultrasound focal size. After the ultrasound is off, the fluid can backflow into the original volume because the source of the hydrostatic pressure is lost, and the solid matrix will recover because of its elasticity. However, as mentioned above, the speed of this recovery only depends on the tissue properties, which may take a much longer time than the time to generate this deformation.

There are three types of motions that can be differentiated. First, the ultrasound pressure wave can induce tissue local oscillations at the ultrasound frequency and its harmonic frequencies, usually at the MHz level. When investigating this type of motion, tissue is considered a single-phase mixture of fluid and solid. It is not necessary to differentiate the fluid from the solid because the frequency is so high that both fluid and solid will oscillate at the same velocity.

Second, the ultrasound pressure wave can also induce much slower motions compared with the MHz oscillations, such as the ARF-induced tissue displacement, a translational motion, or a shear wave motion induced by a pulsed or oscillated ARF, which is a low-frequency oscillation. In this type of motion, tissue is also considered a single-phase mixture of fluid and solid because both components move at the same velocity under a net ARF.

Third, the ultrasound pressure can also induce a squeezing-and-recovering (or a compression-and-expansion) motion at a much lower speed than the speed of the MHz oscillation, which generates a relative motion between tissue fluid and solid matrix (i.e., a motion generated and utilized by SIF-TUM methods). In this type of motion, the speed of the tissue fluid motion can be differentiated from that of the solid matrix. In this Example, assuming no void space can be generated, the exudation fluid velocity from the focal volume (W₂) will be assumed to be equal to the negative velocity of the solid matrix (Vₛ), which means W₂=−Vₛ.

All the final equations have been summarized in Table 1, and the related variables, operators, their physical meanings, and the adopted values in the simulations have been listed in Table 2. Each equation is explained in the following paragraphs. Due to the lengthy derivation procedures, the final equations are directly provided herein. The adopted mathematic methods may be found in the references provided in each paragraph.

TABLE 1
Equations.

| | | Ultra- |
|---|---|---|
| | | sound |
| Eq # | Equation | Status |
| 1 | ${∇}^{2} {P}_{hs} \approx \frac{( {ϕ}_{f} / {ϕ}_{s} )}{{ρ}_{0} K} ∇ \cdot 〈 {ρ}_{1} {V}_{1} 〉 \approx - \frac{α ( {ϕ}_{f} / {ϕ}_{s} )}{{c}_{0} K {ω}^{2} {ρ}_{{0}^{2}}} [ ∇ {P}_{1} \cdot ∇ {P}_{1}^{*} ]$ | On |
| 2a | ${W}_{2} = - K ∇ {P}_{h s} ; \qquad {W}_{2} = - {V}_{s} ; \qquad {u}_{s} = {\int }_{0}^{{t}_{0}} {V}_{s} d t$ | On |
| 2b | $KH {∇}^{2} {u}_{s} = \frac{\partial {u}_{s}}{\partial t} = {V}_{s} ; {W}_{2} = - {V}_{s}$ | Off |
| 3a | ${ρ}_{0} {c}_{t} \frac{\partial ( Δ T )}{\partial t} = {k}_{t} {∇}^{2} ( Δ T ) - {ω}_{b} {ρ}_{b} {c}_{b} ( Δ T ) + \frac{α {\vert {P}_{1} \vert}^{2}}{{ρ}_{0} {c}_{0}}$ | On |
| 3b | ${ρ}_{0} {c}_{t} \frac{\partial ( Δ T )}{\partial t} = {k}_{t} {∇}^{2} ( Δ T ) - {ω}_{b} {ρ}_{b} {c}_{b} ( Δ T )$ | Off |
| 4 | $\frac{\partial c}{\partial t} + ∇ \cdot J = {σ}_{s} - {σ}_{i} ;$ | On & Off |
| | J = − D_{BD}∇C − CD_{TP}∇(ΔT) + CR_{f}W₂ | |

TABLE 2
Major variables, operators, physical meanings and adopted values in simulations.

| Quantity & | | |
|---|---|---|
| Operator | Physical Meaning | Typical value (unit) |
| ∇² | Laplace operator | N/A |
| Pₕₛ | Ultrasound-induced increase of the hydrostatic | Dependent variable |
| | pressure of the interstitial fluid in tissue (2^{nd}-order | (Pa) |
| | small quantity) | |
| ϕ_{f} | Volume fraction of interstitial fluid in tissue | 0.2 (no unit) |
| ϕₛ | Volume fraction of solid matrix in tissue (=1 − ρ_{f}) | 0.8 (no unit) |
| ρ₀ | Average tissue density (0-order component of the | 1,064 (kg/m³) |
| | density) | |
| ρ_{b} | Average blood density (0-order component of the | 1,060 (kg/m³) |
| | density) | |
| K | Tissue (or tumor) hydraulic conductivity (related to | 4 × 10⁻¹⁴ (m⁴/N/s) |
| | tissue permeability and fluid viscosity) | (Tumor) |
| ∇• | Divergence operator | N/A |
| ρ₁ | 1^{st}-order oscillation of tissue density (caused by the | Dependent variable |
| | ultrasound 1^{st}-order pressure oscillation) | (kg/m³) |
| V₁ | Velocity (caused by the ultrasound 1^{st}-order pressure | Dependent variable |
| | oscillation) | (m/s) |
| <ρ₁V₁> | Time average in one oscillation cycle of the | Dependent variables |
| | momentum in unit volume (ρ₁V₁) caused by | ρ₁: kg/m³ |
| | ultrasound | V₁: m/s |
| α | Tissue ultrasound absorption coefficient. | 0.58 (db/MHz/cm) |
| | | or 0.58/8.686 |
| | | (Np/MHz/cm) |
| | | (such that when the |
| | | frequency is 1 MHz, |
| | | the value is 0.58 |
| | | (db/cm)) |
| c₀ | Sound speed in tissue | 1540 (m/s) |
| ω | Angular frequency of the adopted ultrasound | 2π × 2.5 (MHz) |
| P₁ | 1^{st}-order complex amplitude of the ultrasound | Dependent variable |
| | pressure | (Pa) |
| ∇P₁ | Gradient of P₁ (a vector) | Dependent variable |
| | | (Pa/m) |
| ∇P*₁ | Complex conjugate of ∇P₁ (a vector) | Dependent variables |
| | | (Pa/m) |
| • | Vector dot product | N/A |
| cₜ | Specific heat capacity of tissue | 4200 (J/kg/K) |
| c_{b} | Specific heat capacity of blood | 3780 (J/kg/K) |
| kₜ | Tissue thermal conductivity | 0.6 (W/m/K) |
| ω_{b} | Blood perfusion rate | 0.189 (1/s) |

### C. Equations

### Equation 1—Ultrasound Induces Interstitial Fluid Hydrostatic Pressure Increase (P_{Hs}) Via Ultrasound Momentum Transfer

Briefly, the ultrasound pressure wave P(r, t), ultrasound-induced tissue density ρ(r, t), and tissue oscillation velocity V(r, t) can be expressed as follows:

$$P ( r , t ) = {P}_{0} ( r ) + {P}_{1} ( r ) exp ( - i ω t ) + {P}_{2} ( r ) exp ( - i ω t ) + c . c . \qquad ( 0 a )$$

$$a . ρ ( r , t ) = {ρ}_{0} ( r ) + {ρ}_{1} ( r ) exp ( - i ω t ) + {ρ}_{2} ( r ) exp ( - i ω t ) + c . c . \qquad ( 0 b )$$

$$b . V ( r , t ) = {V}_{1} ( r ) exp ( - i ω t ) + {V}_{2} ( r ) exp ( - i ω t ) + c . c . \qquad ( 0 c )$$

Here, r and t represent the location and time; the subscripts of 0, 1, and 2 indicate the 0th, 1st, and 2nd order of the quantities, respectively; ω=2πf is the ultrasound angular frequency, and f=2.5 MHz in this study; c.c. is a complex conjugate. The above representation is commonly used in the literature to separate the time and space variables for sinusoidal oscillations. All the symbols of r in the parentheses on the right hand side of the three equations will not be discussed in the following sections, but these are functions of r but are independent of time. Any quantities or their multiplications with an order higher than 2nd were ignored.

Thus, inserting these variables into the mass conservation (i.e. continuity equation) and Navier-Stokes equation (eventually reduced to Darcy's law equation), the first key equation was derived:

$${∇}^{2} {P}_{h s} \approx \frac{( {ϕ}_{f} / {ϕ}_{s} )}{{ρ}_{0} K} ∇ \cdot 〈 {ρ}_{1} {V}_{1} 〉 \approx - \frac{α ( {ϕ}_{f} / {ϕ}_{s} )}{{c}_{0} K {ω}^{2} {ρ}_{0}^{2}} [ ∇ {P}_{1} \cdot ∇ {P}_{1}^{*} ]$$

(i.e., Equation 1 in Table 1). Pₕₛ is a 2nd-order small quantity and represents the ultrasound-induced increase of local hydrostatic pressure of the interstitial fluid at a specific location in the tissue.

This equation is an important result. It indicates that Pₕₚ is generated by the divergence of the temporal average of the 1st-order momentum density of the ultrasound wave (i.e., ∇·(ρ₁V₁)). Here, ρ₁V₁ is the 1st-order momentum density of the ultrasound wave at a specific location and time. ( ) represents the temporal average in one ultrasound oscillation cycle. Physically, because the ultrasound wave loses momentum and transfers it to tissue, ∇. (ρ₁V₁) should be a negative value. This momentum transfer from ultrasound to tissue fluid leads to a local hydrostatic pressure rise (Pₕₛ) of the tissue fluid because of a finite value of the hydraulic conductivity of tissue (K). If K was an infinite large value, Pₕₚ would be zero because K is in the denominator.

Further, the term of

$$\frac{( {ϕ}_{f} / {ϕ}_{s} )}{{ρ}_{0} K} ∇ \cdot 〈 {ρ}_{1} {V}_{1} 〉$$

can be expressed as

$$- \frac{α ( {ϕ}_{f} / {ϕ}_{s} )}{{c}_{0} K {ω}^{2} {ρ}_{0}^{2}} [ ∇ {P}_{1} \cdot ∇ {P}_{1}^{*} ]$$

(see Equation 1) using a similar method. It involves the tissue ultrasound absorption coefficient (α), the volume fraction ratio of the tissue interstitial fluid and solid matrix (ϕ_{f}/ϕₛ), the sound speed (c₀), the hydraulic conductivity of tissue (K), the square of tissue's 0th order density (ρ₀²), the square of the ultrasound angular frequency (ω²), and ∇P₁ (the gradient of the 1^{st} order complex amplitude of the ultrasound pressure P₁). ∇P₁ is a vector. ∇P₁* is the complex conjugate of ∇P₁ and is also a vector. The symbol of · is the dot product between the two vectors. This result clearly shows that Pₕₛ is determined by [∇P₁·VP₁*] rather than the pressure itself. [∇P₁·∇P₁*] can be further represented as

$$[ {( \frac{\partial R}{\partial x} )}^{2} + {( \frac{\partial I}{\partial x} )}^{2} ] + [ {( \frac{\partial R}{\partial y} )}^{2} + {( \frac{\partial I}{\partial y} )}^{2} ] + [ {( \frac{\partial R}{\partial z} )}^{2} + {( \frac{\partial I}{\partial z} )}^{2} ] ,$$

where R and I are the real and imaginary parts of P₁, respectively. Because each term is squared, [∇P₁·∇P₁*] is positive. Therefore, the negative sign in front of the right-hand part of Equation 1 further indicates that ∇· [symbol] ρ₁V₁ [symbol] is negative.
Equation 2a—the Gradient of Elevated Hydrostatic Pressure (∇Pₕₛ) induces the exudation fluid velocity (W₂)

Once Pₚₛ is calculated from Equation 1 based on Darcy's law, the exuding velocity of the fluid from the focal volume can be calculated using the following equation: W₂=−K∇Pₛₚ=−Vₛ, where K is the tissue's hydraulic conductivity. Once W₂ is calculated, the velocity of the solid matrix (Vₛ) is known. The squeezing-caused displacement (i.e. compression) uₛ can be integrated from t=0 to t₀ (ultrasound exposure time) using the equation of

$${u}_{s} = {\int }_{0}^{{t}_{0}} {V}_{s} d t ,$$

as shown in Equation 2a. Equation 2a shows that the elevation of the hydrostatic pressure (Pₕₛ) in the ultrasound focal volume will lead to the exudation of the fluid from the focal volume with an outward velocity of W₂ (a positive value), and therefore, it will lead to the compression of the tissue solid matrix with an inward velocity Vₛ (a negative value). Depending on the value of [∇P₁·∇P₁*], a typical value of this flow velocity can reach above a few microns/second(s), which is much higher than the typical value of the natural flow velocity in a tumor (<1 micron/s). The velocity of this flow is controllable via [∇P₁·∇P₁*], which functions as a convection transport mechanism and brings the agents out of the focal volume. One can imagine it as a pumping procedure when ultrasound is turned on. This is much more efficient and controllable than altering tissue permeability and diffusivity as per the conventional methods.
Equation 2b—Turning Off Ultrasound Leads to a Backflow of the Fluid (W₂) Because of the Recovery of the Solid Matrix

When ultrasound is turned off, the driving force is removed (i.e., [∇P₁·∇P₁*]=0), and Pₕₛ decays to zero almost immediately because of the extremely fast decay speed of the hydrostatic pressure relaxation. The compressed solid matrix tends to expand and recover to its original position at a velocity of Vₛ because of its elasticity, as described by an apparent modulus H. This expansion will suck the fluid back into the focal volume because any expanded volume must be filled by fluid, assuming no void space occurs in tissue. The velocity of tissue solid matrix can be described via a bi-phase model, which can be expressed as

$$K H {∇}^{2} {u}_{s} = \frac{\partial {u}_{s}}{\partial t} = {V}_{s}$$

and listed as Equation 2b in Table 1. uₛ is the displacement of the solid matrix from its original position, and Vₛ is its velocity. The initial spatial distribution of uₛ can be calculated from the previous step in Equation 2a. Once uₛ is calculated, Vₛ can be calculated based on

$${V}_{s} = \frac{\partial {u}_{s}}{\partial t} .$$

The recovery velocity of Vₛ at the early stage is usually high because of tissue's high apparent modulus (H). Once Vₛ is calculated, W₂ is known based on W₂=−Vₛ in Equation 2b.

The simulation results described herein showed that a portion of the fluid can quickly flow back into the original focal volume, but the rest of fluid will flow back slowly. This is notable and favorable for controlling agent distribution. First, quickly recovering tissue fluid allows this procedure to be repeated without mechanically damaging tissue because of the significant loss of tissue fluid in the focal volume. Second, the slow backflow avoids a complete recovery of the agents, and therefore, it allows continuously pumping the agents out of the focal volume at a future time via an accumulation effect.

Equation 3—Ultrasound Induces a Tissue Temperature Rise (ΔT) Because of the Tissue Absorption of Acoustic Energy and ΔT Decays when Ultrasound is Off

For safety reasons, and if desired, the maximum temperature increase ΔT can be limited to a preset value of ΔT₀, which is eventually regulated by a thermal index. Also, the gradient of ΔT can affect agent distribution via thermophoresis. It is possible to calculate ΔT using a bio-heat transfer equation, which is Equation 3a in Table 1. When ultrasound is off, the decay of ΔT is described via Equation 3b by setting the amplitude of P₁ equal to zero (i.e., |P₁|=0). In Equation 3, cₜ and c_{b} are the specific heat capacity of tissue and blood, respectively. kₜ is the tissue thermal conductivity. ρ₀ and ρ_{b} are the density of tissue and blood, respectively. ω_{b} is the blood perfusion rate. c₀ is the speed of sound in tissue.

Equation 4—Ultrasound Induces Agent Concentration Change from Three Different Mechanisms

After W₂ and ΔT are calculated from Equations 2 and 3, the dynamic change of agent concentration can be calculated based on the continuity equation (i.e., Equation 4 in Table 1). C is the agent concentration. J is the total flux due to agent transport via three mechanisms, including diffusion because of concentration gradient (−D_{BD}∇C), thermophoresis because of ultrasound-induced temperature gradient (−CD_{TP}∇(ΔT)), and the SIF-TUM-induced flow of interstitial fluid (CR_{f}W₂). σₛ and σᵢ are the rate of source and sink of the agents.

In this Example, for simplicity, it is assumed σₛ=σᵢ=0. This means no extra sources and sinks can create or destroy the agents during the investigation time period. D_{BD} and D_{TP} are the agent diffusion coefficient and thermophoresis diffusion coefficient, respectively. The agent diffusion coefficient D_{BD} is calculated via the equation of

$${D}_{BD} = \frac{{k}_{B} T}{6 π μ R} ,$$

in which k_{B} is the Boltzmann constant, T is the temperature in Kelvin, μ is the viscosity of the intestinal fluid (1×10⁻³ Pa*s), and R is the radius of the agent. The thermophoresis diffusion coefficient D_{TP} is calculated via the equation of D_{TP}=S_{T}D_{BD}, in which S_{T} is the Soret coefficient (=0.01 Kelvin⁻¹). R_{f} is the retardation factor that has been defined as the ratio of the velocity of the solute (i.e., agent) to its solvent (i.e., interstitial fluid). In this Example, R_{f} was used to reflect the effect of agent size on transportability. Briefly, R_{f} was calculated by comparing the average size of the agents and the pore size of the tissue extracellular matrix. If agents have a size much smaller than the pore size, R_{f} is close to 1. If the agent size is close to or larger than the pore size, R_{f} will significantly reduce.

### D. Simulations

### Simulation of P₁ of Ultrasound Pressure Wave

In this Example, an analytical method was adopted to simulate the complex pressure amplitude P₁ (i.e., P₁(r) in Equation 0a), which is expressed as follows:

$${P}_{1} ( x , y , z ) = \frac{{P}_{a} {z}_{d}}{2 {sinh}^{2} ( {kz}_{d} )} [ {e}^{{kz}_{d}} \frac{sin ( k \sqrt{{D}_{-}} )}{\sqrt{{D}_{-}}} - {e}^{- {kz}_{d}} \frac{sin ( k \sqrt{{D}_{+}} )}{\sqrt{{D}_{+}}} ] \qquad ( 5 )$$

where Pₐ is a scalar used to control the spatial peak amplitude value of P₁(x, y, z) at the focal center,

$${z}_{d} = k {a}^{2} / 2 , k = \frac{2 π}{λ} ,$$

λ is the ultrasound pressure wavelength, and a is a parameter affecting the lateral size of the beam in the plane z=0. In this Example, a was assigned a value equal to the lateral full-width-at-half-maximum (FWHM) of the ultrasound focus: D₋=x²+y²+(z−jz_{d})², D₊=x²+y²+(z+jz_{d})², and j=\sqrt{−1}. In this Example, the ultrasound frequency is 2.5 MHZ, and the FWHM of the focus along the lateral (x and y) and axial direction (z, i.e., the wave propagation direction) is σₓ=σ_{y}=0.5 and σ_{z}=3.5 mm, respectively.

### Numerical Simulations

Some of the equations in Table 1 involve a diffusion term (∇²). Equations 1 can be described as Poisson's equation. Equation 2b and 3 are diffusion equations, and Equation 4 is a continuity equation eventually involving two diffusion terms. This type of equation can be numerically solved by using a finite difference method with appropriate initial and boundary conditions. When time is involved in the simulation, the stability condition has been well identified as

$$Δ t < \frac{{( Δ l )}^{2}}{2 D} .$$

Here, Δt is the step size in time, and Δl is the step size in space. If a Cartesian coordinate system is adopted, Δl represents Δx, Δy, or Δz. D represents the diffusion coefficient, such as kₜ/ρ₀cₜ in Equation 3, when the diffusion equation has a format of

$$\frac{\partial F}{\partial t} = D {∇}^{2} F$$

(here r is a spatial and temporal variable). In this Example, a Cartesian coordinate system was adopted. To show the data in the focal volume, the origin of the coordinate system was selected at the center of the ultrasound focus. When boundary conditions were needed, zero-gradient boundary conditions were adopted (i.e.,

$$\frac{\partial F}{\partial l} = 0 ,$$

where l can be x, y, or z). This is because the boundaries are far away from the ultrasound focus by adopting a simulation volume that is much bigger than the focal volume. In Equation 2a, the initial condition for

$${u}_{s} = {\int }_{0}^{{t}_{0}} {V}_{s} dt$$

was uₛ|ₜ₌₀=0, and its final value of uₛ|ₜ₌ₜ_{₀}=uₛ₀ was used as the initial condition for

$$\frac{\partial {u}_{s}}{\partial t} = KH {∇}^{2} {u}_{s}$$

in Equation 2b. Other initial conditions include ΔT=0|ₜ₌₀ and C=C₀|ₜ₌₀ (usually the concentration C is normalized by C₀).

### E. Results and Discussion

### “Squeezing” Tissue's Interstitial Fluid Out of the Focal Volume Via Ultrasound Momentum Transfer

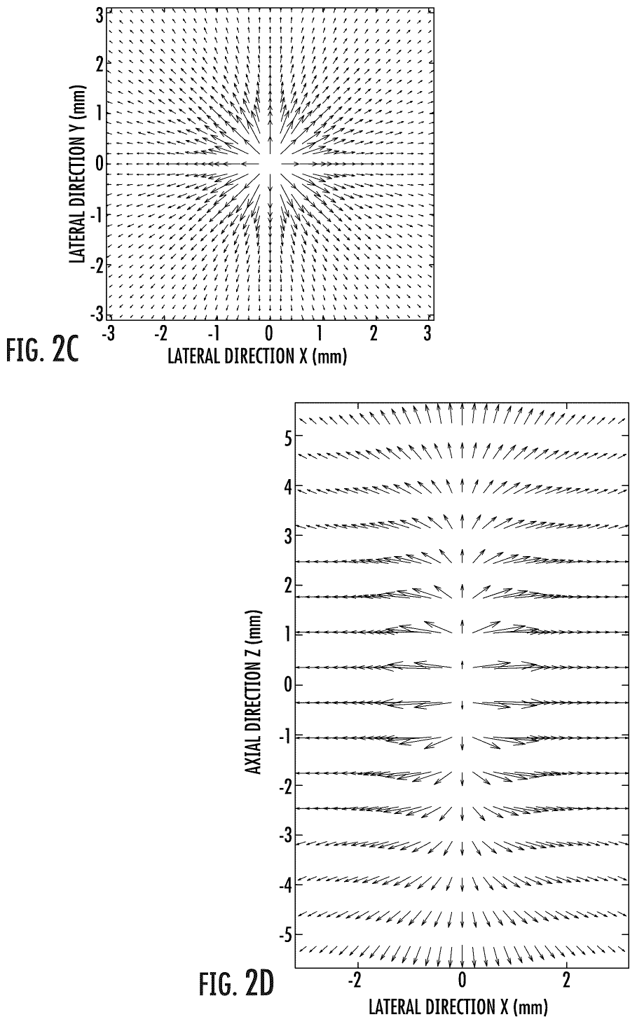

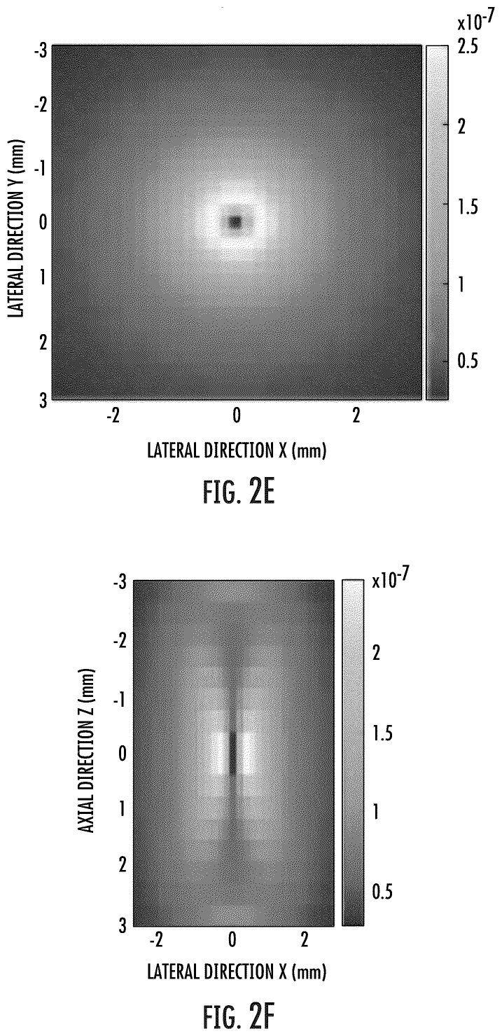

FIG. 2A shows the 2D distribution of the amplitude of the 1st-order ultrasound pressure wave (i.e., |P₁|=\sqrt{R²+I²}) on the xz (y=0) plane with a central peak value of Pₐ=1 MPa. The dashed box in FIG. 2A indicates the focal area. Again not intending to be bound by theory, it is believed the momentum transferred from the ultrasound wave to the tissue leads to the interstitial fluid being “splashed” away from the focal volume (FIG. 2C-D). It further leads to an elevation of the hydrostatic pressure (Pₕₛ) of the interstitial fluid. Meanwhile, the tissue solid matrix will be volumetrically compressed (data not shown). FIG. 2B shows the distribution of the elevated fluid pressure Pₕₛ on the xz plane (y=0) induced by the ultrasound beam in FIG. 2A. Based on Darcy's law, Pₕₛ will generate an exudation flow W₂ (FIG. 2C-D). Thus, a small amount of tissue interstitial fluid will be squeezed out of the focal volume, which will also move the agents (i.e., the solutes in the fluid) out of the focal volume. FIG. 2C shows the x and y components of W₂ on the xz plane (z=0). FIG. 2D shows its x and z components on the xz plane (y=0). Each arrow points to the flow direction, and its length is proportional to the flow speed. FIG. 2E-F display the magnitude of W₂ (i.e., [W₂]) on the xy (z=0) and xz (y=0) planes, respectively. The velocity can reach as high as 0.25 microns/s in this Example at the positions surrounding the center point, where the gradients of P₁ and Pₕₛ (i.e., ∇P₁ and ∇Pₕₛ) achieve the maxima. Pₕₛ is diffused in space because it is governed by a Poisson-like equation (i.e., Equation 1 in Table 1). Thus, the distribution of Pₕₛ will be spatially expanded compared with that of |P₁|. This can be seen by comparing the lateral and axial FWHMs of Pₕₛ in FIG. 2B (3.6 and 10.5 mm) with those of | P₁| in FIG. 2A (0.5 and 3.5 mm), which may be favorable for transporting agents in a relatively large volume.

Backflows when Ultrasound is Off

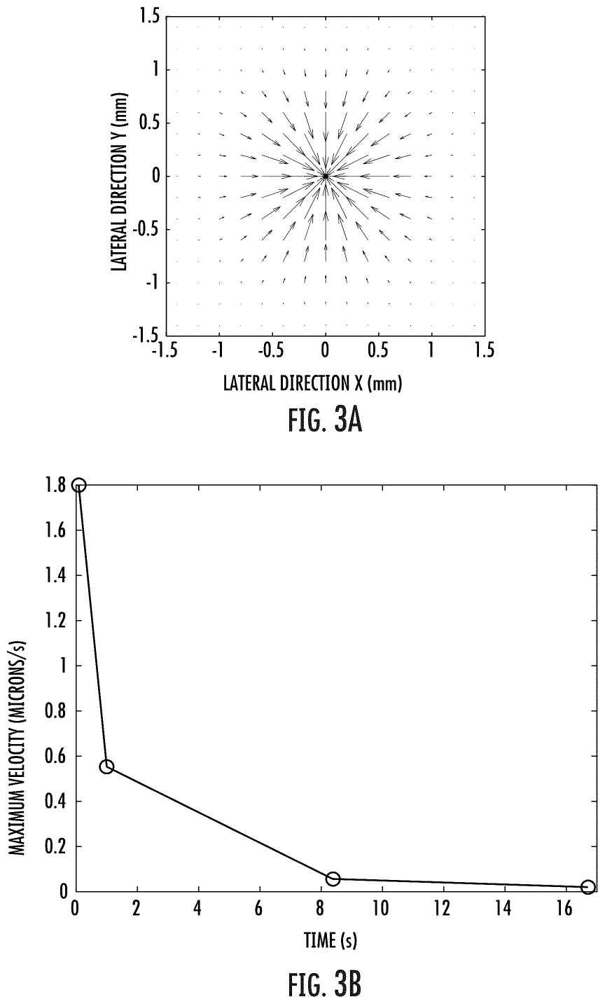

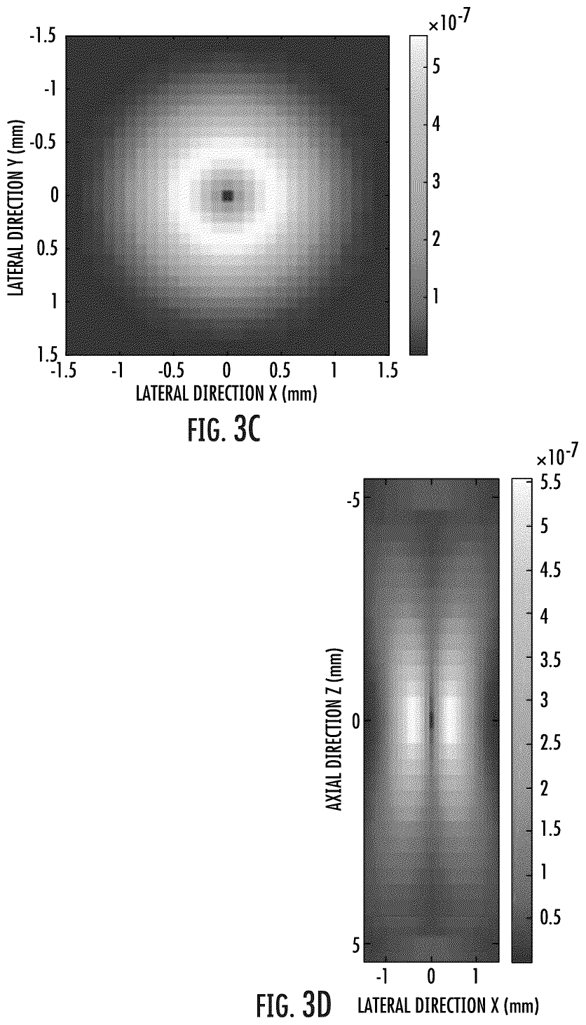

FIG. 3A shows the backflow velocity (W₂) on the xy plane (z=0) at the time of 1 s after the ultrasound is off. The fluid is flowing back into the focal volume. FIG. 3C-D show the corresponding spatial distribution of the magnitude of the backflow velocity (|W₂|) on the xy (z=0) and xz (y=0) planes at t=1 s, respectively. FIG. 3A displays the maximum backflow velocity as a function of time after the ultrasound is off. The initial maximum velocity can reach as high as 1.8 microns/s and quickly reduces to 0.06 microns/s at t of 8.4 s. Then, the velocity decays slowly and remains a small value for a long time.

For safety, the mechanical index (MI) of the ultrasound can preferably remain below the FDA-required safety threshold (MI<1.9) and the thermal indexes (TI) below the AIUM-required safety threshold (TI<6; American Institute of Ultrasound in Medicine). Thus, this procedure can be safely repeated multiple times at each location when needed. The total amount of the squeezed-out agents will be accumulated. Because the size of the ultrasound focus is small, the spatial distribution of agents can be controlled by 3-dimensionally scanning a single focus or multiple foci generated via an array transducer.

### Temperature Rise Induced by Ultrasound

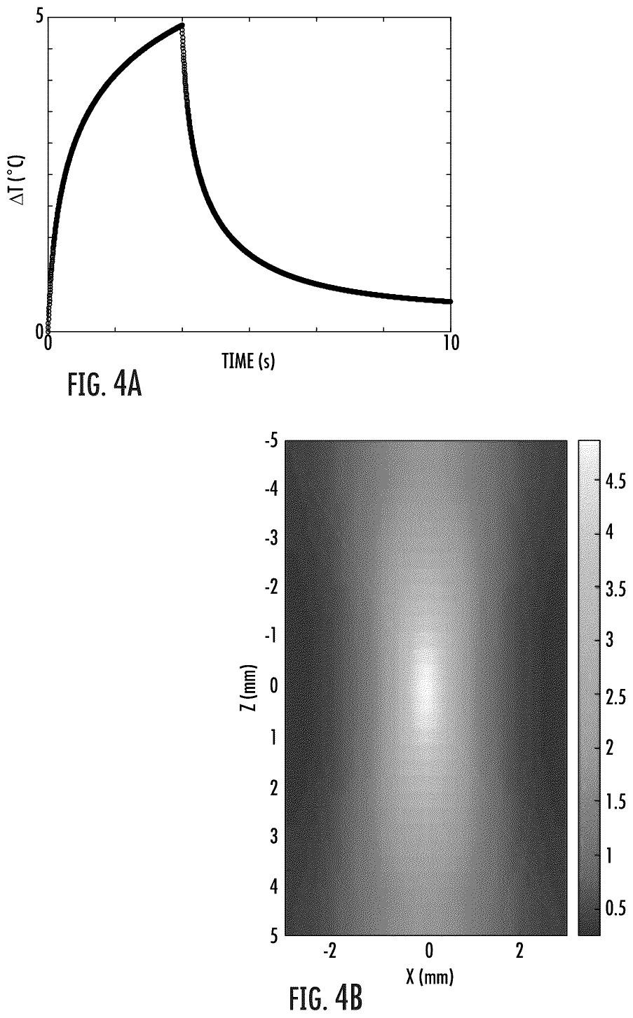

As an example, FIG. 4A shows the change of ΔT as a function of time at the center of the ultrasound focus with an exposure time of t₀=10 seconds and an ultrasound spatial peak amplitude Pₐ=1 MPa and α=0.58 db/MHz/cm. Clearly, during the period between 0 and 10 s when the ultrasound is turned on, ΔT quickly rises from 0 to 4.87° C., but the increase rate is gradually reduced, e.g., because of thermal diffusion and blood perfusion (see the first term and the second term on the right hand side of Equation 3a in Table 1). FIG. 4B shows the 2D distribution of ΔT on the xz plane (y=0) when t=t₀=10 seconds. The lateral (x) and axial (z) FWHMs of ΔT are 1.4 and 6.5 mm, respectively, which are wider than those of |P₁| in FIG. 2A (0.5 and 3.5 mm, respectively), because of the existence of thermal diffusion (again, not intended to be bound by theory).

### Concentration Change Induced by Ultrasound

In this section, both temperature-insensitive and -sensitive agents are described. Here, for reference purposes, a temperature-insensitive agent means the temperature change does not affect the size or other transport-related parameters of the agent. Conversely, a temperature-sensitive agent, such as a USF contrast agent, indicates the diameter of the agent is significantly reduced when temperature is above its phase transition threshold, and the size recovers when the temperature falls below the threshold.

### Temperature-Insensitive Agents

For the simplicity of the simulation, both agents and tissue pores are assumed to be spherical, and their diameters are assumed to have Gaussian distributions. However, these characteristics are not required. The standard deviations of the diameter distributions are arbitrarily selected to be 25% of their average diameters, which are also not required characteristics. For example, if the average diameters of the agent and the tissue pore size are set as 40 and 80 nm, respectively, and the 25% standard deviation of their diameter distributions are 10 and 20 nm, respectively, then the majority of the agents will have a size smaller than that of the majority of the pores. Therefore, the agents should be able to pass through the pores without significant resistance. Whenever the following parameters are constant, their values will be set as follows: the spatial peak pressure value is Pₐ=0.8 MPa; the ultrasound exposure time is 40 s. Based on the above two parameters, the spatial peak temperature increases ΔT=4.15° C. if the tissue ultrasound absorption coefficient is α=0.58 db/MHz/cm; the hydraulic conductivity is K=4×10⁻¹⁴ m⁴/N/S; and the apparent module is H=1 MPa. As will be appreciated, tissue ultrasound absorption coefficient is a function of angular frequency and its unit can be 1/m in equations 1 and 3a after considering the frequency effect.

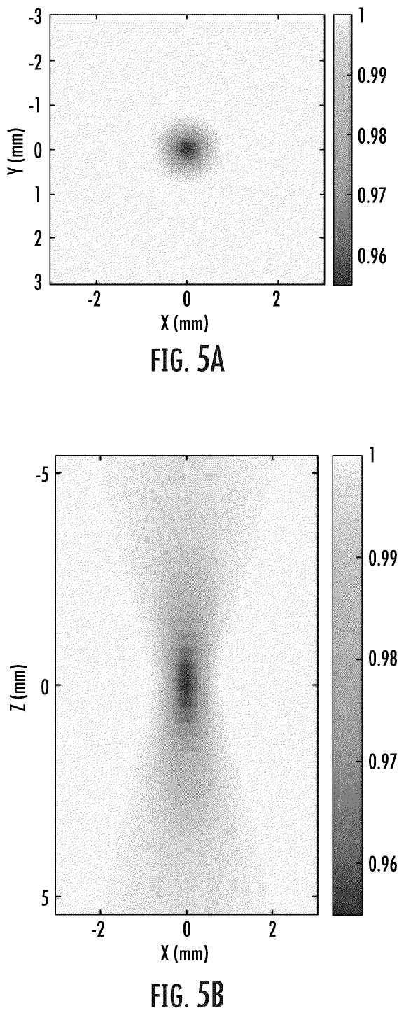

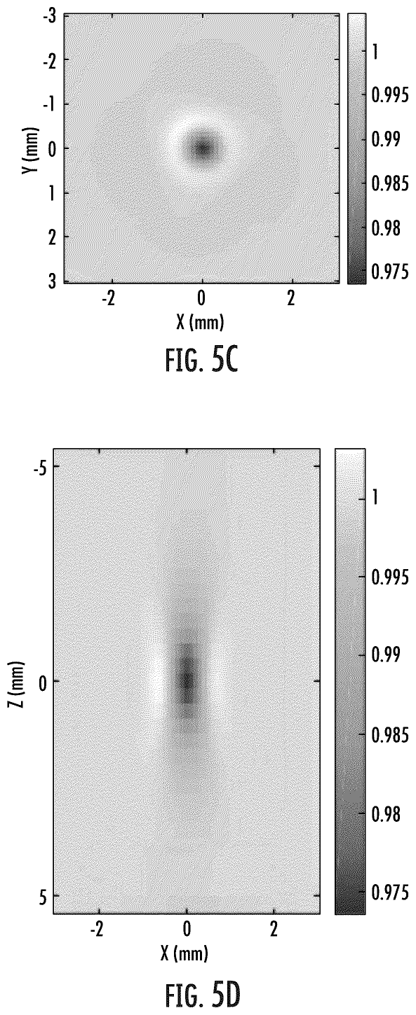

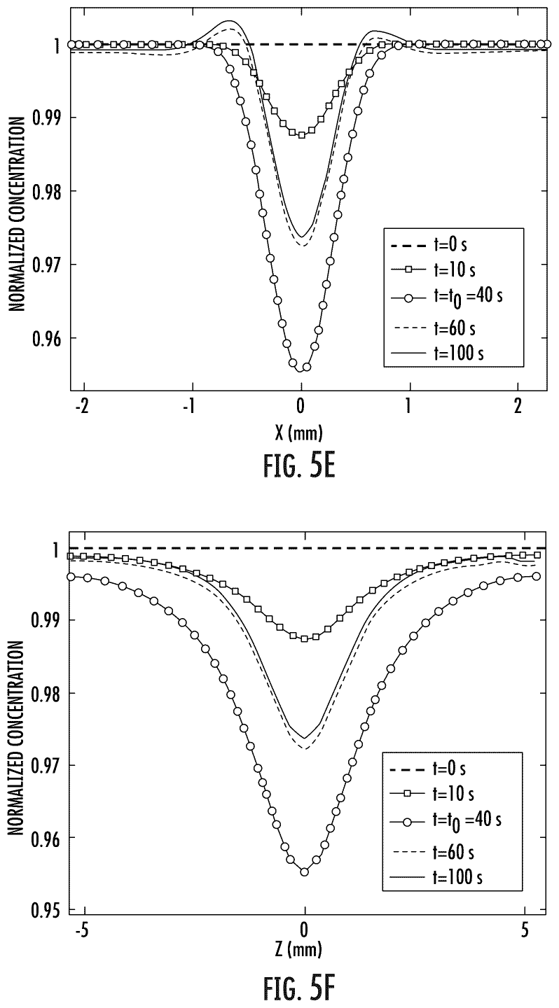

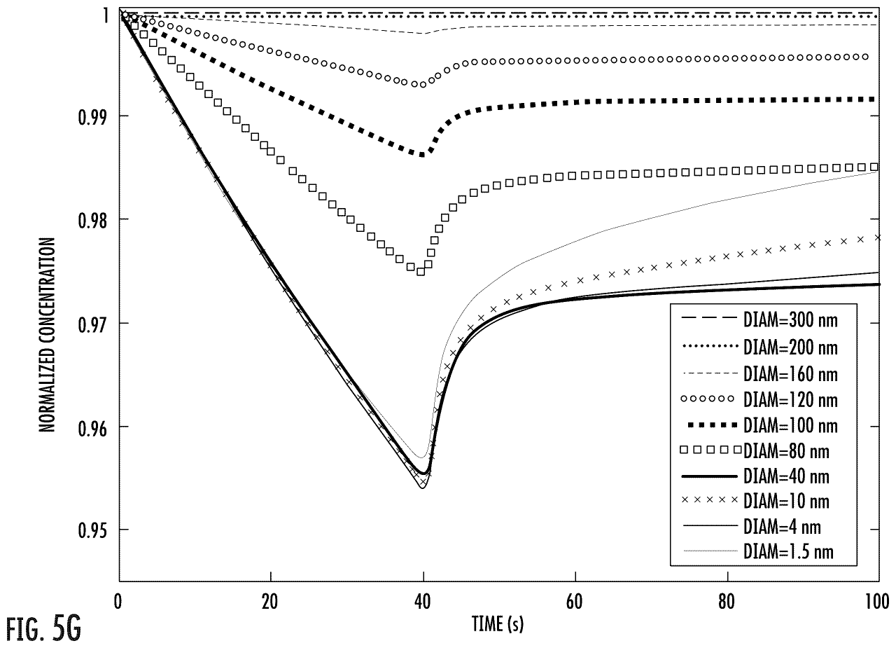

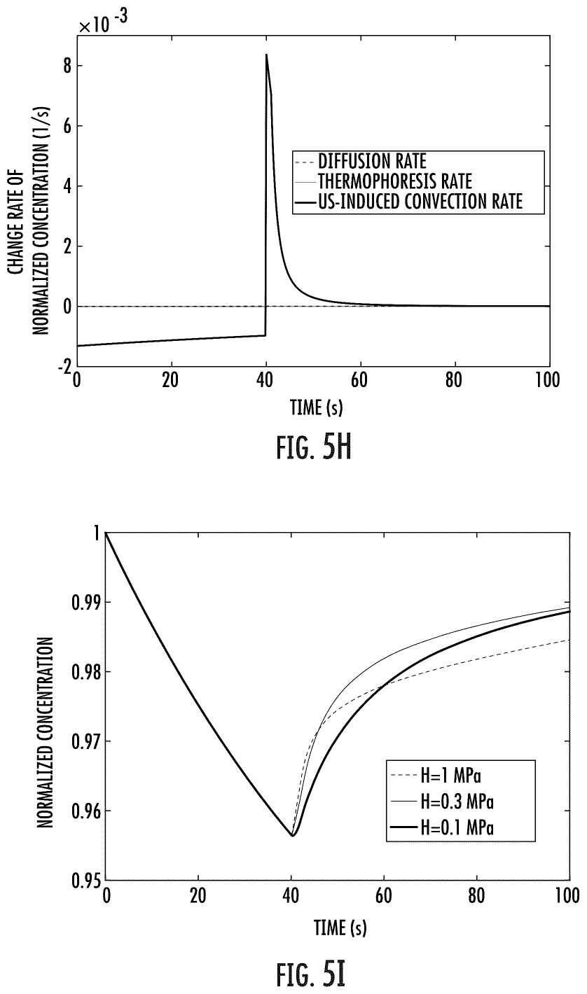

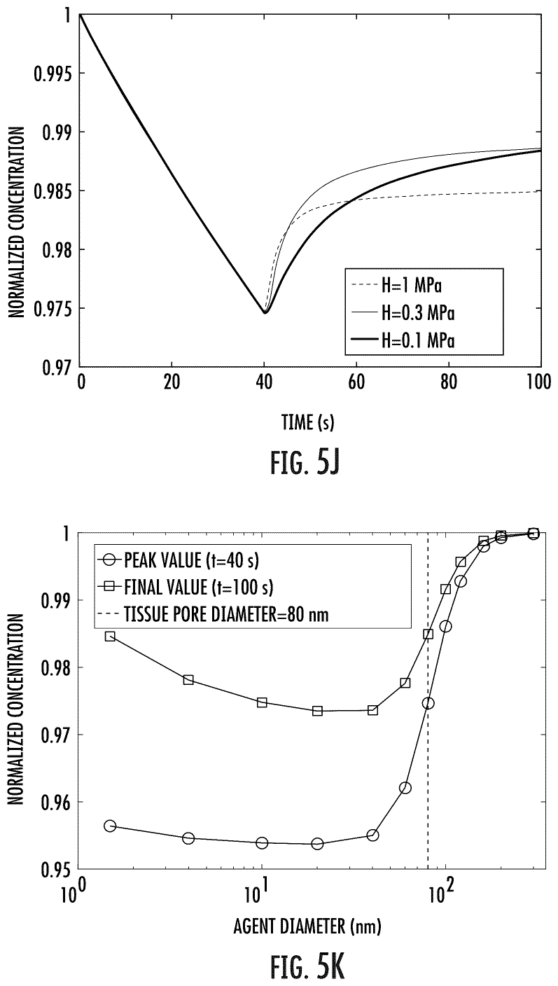

FIG. 5A-B show the change in the normalized concentration of the agent (C/C₀) in the xy plane (z=0) and the xz plane (y=0) at the time right after ultrasound is off, respectively (i.e. t=t₀=40 s). FIG. 5C-D show the results at the time of 60 seconds after ultrasound is turned off (i.e. t=t₀+60=100 s). FIG. 5E-F plot the normalized concentration changes along the x and z directions, respectively, at different times. FIG. 5G shows the dynamic change of the normalized concentration as a function of time at the ultrasound focal center with different agent average diameters. FIG. 5H displays the variation rates of the normalized concentration caused by different mechanisms: diffusion (i.e. ∇·[(D_{BD}∇C)]), thermophoresis (i.e., ∇·[CD_{TP}∇(ΔT)], and ultrasound-induced convection rate (i.e. −∇·[CR_{f}W₂]). FIG. 5I-J show the effect of the tissue apparent module (H) on the relaxation of the normalized concentration change at the center of the focus after ultrasound is off. The agent average diameter is 1.6 nm and 80 nm in FIG. 5I-J, respectively. FIG. 5K displays the effect of the agent average diameter on the normalized concentration at 40 and 100 s.

From the above results, it can be seen that during the period when the ultrasound is on (0≤t≤40 s), the agents are continuously squeezed out of the focal volume, as shown in FIG. 5A-G. The concentration in the surrounding areas should increase (i.e., C/C₀>1), but it may not be visible in FIG. 5A-B because of the large surrounding volume. The maximum concentration reduction at the center of the ultrasound focus can reach ˜4.5% (i.e., reduced from 100% to 95.5%) at 40 s for the agent with a diameter of 10 nm as shown in FIG. 5G. Other agents with a diameter smaller than the pore diameter of 80 nm, such as 40, 4, and 1.5 nm, can also achieve a similar concentration change. This can be understood by considering that these nano-scale agents have similar R_{f} because of their small size. In the group in which the agent size is smaller than tissue pore size, the agent with 1.5 nm has slightly smaller concentration reduction than those of other agents, and it also has a quicker recovery speed after ultrasound is off. Again, not intending to be bound by theory, it is believed this is mainly because of the relatively large diffusion caused by concentration gradient due to its much smaller size. However, when the agent size is much larger than the pore size, such as 120-300 nm, the maximum concentration change is less than 1% because of the high resistance of tissue to the large agents.

FIG. 5H shows that the SIF-TUM contribution (bold line) is much more significant than those from the diffusion and thermophoresis. Therefore, SIF-TUM is the dominant driving force of the agent transport. During the period when the ultrasound is on (0-40 s), the rate induced by SIF-TUM is negative, which means the concentration in the ultrasound focal volume will be reduced. Once the ultrasound exposure is stopped at 40 s, the rate becomes positive, which means the fluid flows back into the focal volume. Also, during the period when the ultrasound is on, the SIF-TUM rate is almost a constant, as shown in FIG. 5H, although it is slightly reduced because the remaining fluid in the focal volume is slowly reduced when fluid is continuously squeezed out. Therefore, the concentration is approximately reduced linearly as a function of time during this period as shown in FIG. 5G. After the ultrasound is off, the normalized concentration (C/C₀) quickly recovers, reaching a relatively stable value and then decaying extremely slowly, which is shown in FIG. 5G. This can be understood by examining the backflow rate on FIG. 5H. After the ultrasound is off, the backflow rate mainly depends on the properties of the tissue and becomes independent of the ultrasound parameter. The initial rate of the backflow is even greater than the flow rate induced by the ultrasound during the period of ultrasound illumination. However, it quickly decays to a small number, as shown in FIG. 5H, leading to a slow recovery of the concentration of the agents, as shown in FIG. 5G. Based on FIG. 5E-H, the high initial backflow speed (i.e., immediately after the ultrasound is off) leads to a small portion of concentration recovery in the focal volume. However, when t>50 s, the backflow speed becomes so small that the agent concentration recovery is almost unnoticeable during the time frame adopted in this Example, except for the small agents, such as 1.5 and 4 nm in diameter.

Another interesting result is that a ring shape of concentration is observed in FIGS. 5C-D and 5E-F, which means that more agents are accumulated surrounding the focal area after the ultrasound is turned off. Again, not intending to be bound by theory, this may be caused by the initially fast and finally slow backflows, and it may be further related to the spatial phase difference of the backflows between the central and surrounding area of the ultrasound focus.

FIG. 5I-J indicates the tissue relaxation is also slightly affected by the apparent modulus (H). The higher H provides the higher initial speed of backflows and reaches the final value more quickly. Again, the continuous concentration reduction at the late stage in FIG. 5I is believed to be mainly due to the diffusion of the small agent, which is much weaker for large agents as shown in FIG. 5J. FIG. 5K displays how the agent diameter affects the normalized concentration change (C/C₀) at the ultrasound focal center. The line with circles and the line with squares are the values at t=40 and t=100 seconds, respectively. Clearly, agents with a diameter between 10 and 40 nm achieve a preferred concentration reduction when the average diameter of the tissue pores is 80 nm. This means that within this diameter range, the agents not only freely pass through the tissue pores but also avoid significant natural diffusion due to their medium size. When the diameter is larger than 40 nm, especially when larger than 80 nm, the resistance of tissue to the agent transport becomes significant, and the concentration change becomes difficult. On the other hand, when the diameter is smaller than 10 nm, although these agents can easily pass through the pores, they can also diffuse back into the focal volume more efficiently than the larger ones, which reduces the efficiency of concentration change. However, even with this disadvantage of diffusion-caused backflow for small-sized agents, such as 1.5 and 4 nm, their concentration changes are still higher than those of the large-sized agents, such as >80 nm. This means the transportability is more important than the diffusivity in the current setup.

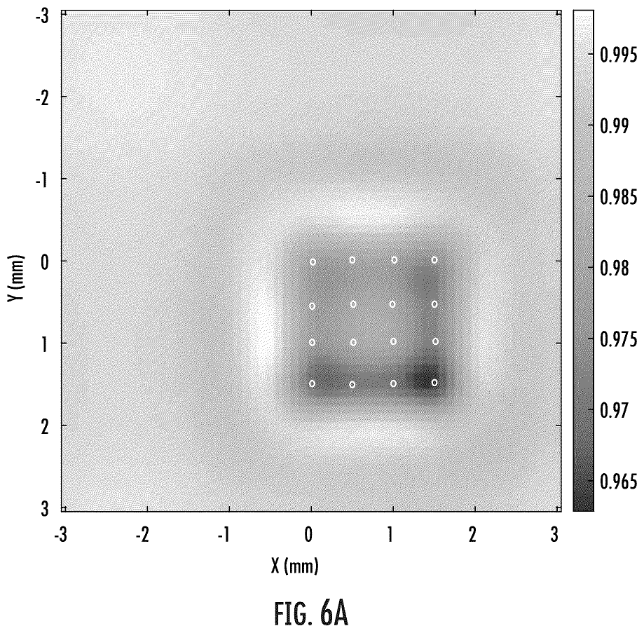

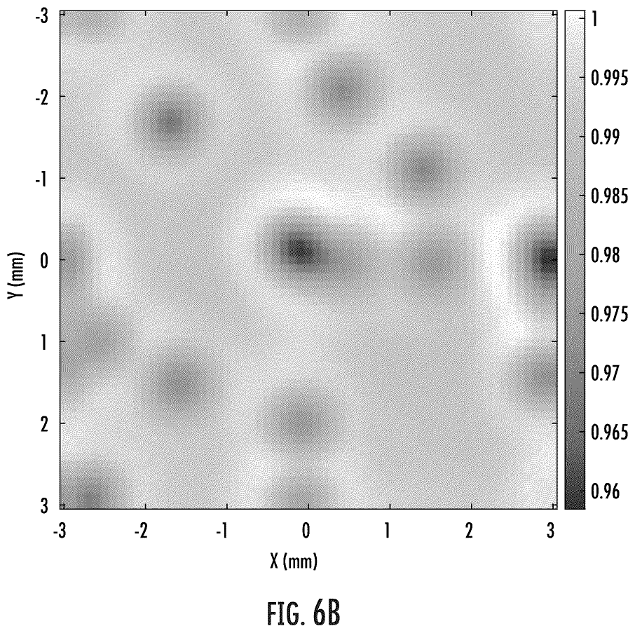

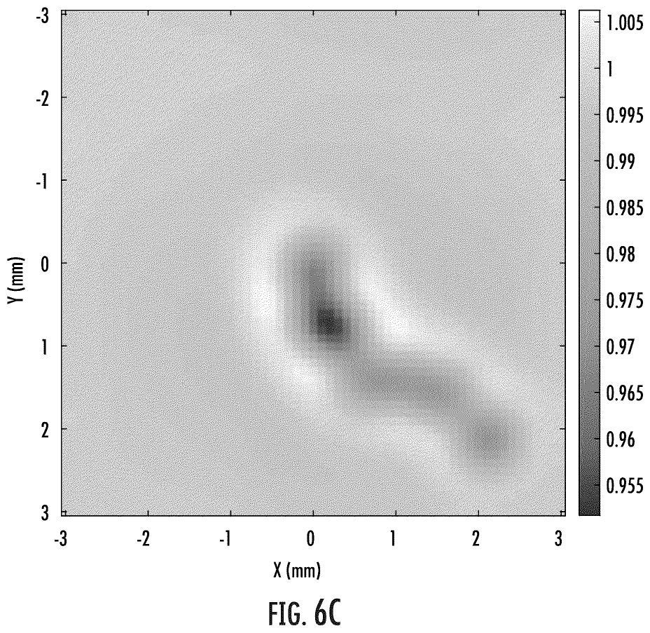

FIG. 6 shows three examples showing how the concentration is changed when multiple ultrasound foci exist. FIG. 6A displays the normalized concentration distribution on the xy plane (z=0) plane when a total of 16 ultrasound foci exist, which may be achieved via either a scanning or an array transducer simultaneously generating multiple foci. The dots indicate the focus locations. The concentration in the scanned area is reduced, and the agents are pushed outside the focal area. This may be used to control the agent distribution externally. FIG. 6B-C display similar results but with two random scans. The concentration of the agents can clearly be controlled by selecting the scanning area. The adopted parameters are similar to those in FIG. 5, including diameter=40 nm; Pₐ=0.8 MPa; t₀=40 s; H=1 MPa; ΔT=4.15° C.; K=4×10⁻¹⁴ m⁴/N/S; and FWHM=0.5, 0.5, and 3.5 mm along x, y, and z, respectively.

### Temperature-Sensitive Agents, Such as USF Imaging Contrast Agents

In some embodiments, the diameters of USF agents can reduce approximately 2 times when the environment temperature is increased above a temperature threshold of the agents (i.e., the lower critical solution temperature of the material, LCST, which is set as 38° C. in this Example, whereas the tissue background temperature is 37° C.). Thus, when the ultrasound is applied and tissue temperature in the focal volume is raised by a few degrees, the agents in the focal volume will shrink. The size reduction leads to the increase of R_{f}, which is favorable for the agents to be transported via the SIF-TUM-induced flow. After the agents are squeezed out, the agents will expand to their original size. This leads to the reduction of R_{f}, which is unfavorable for agents to flow back after the ultrasound is off. Compared with temperature-insensitive agents, this feature can be favorable for enhancing the transport efficiency and can enable the transport of agents with a size larger than the pore size.

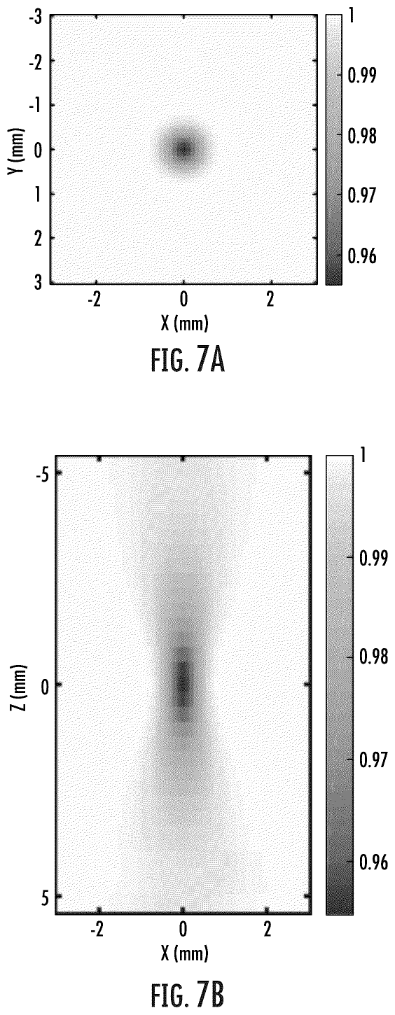

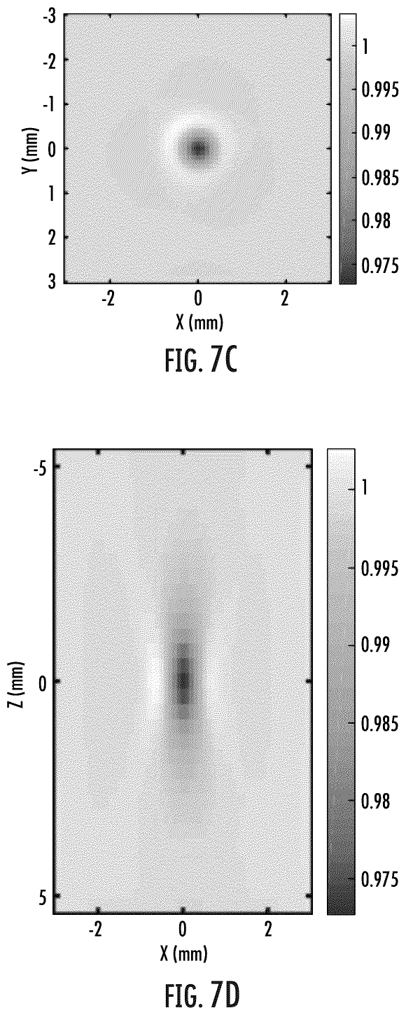

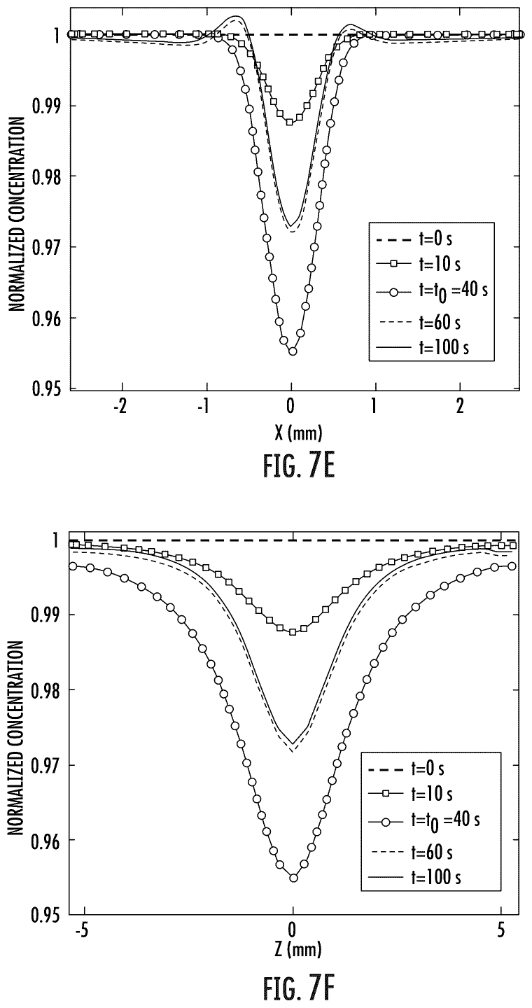

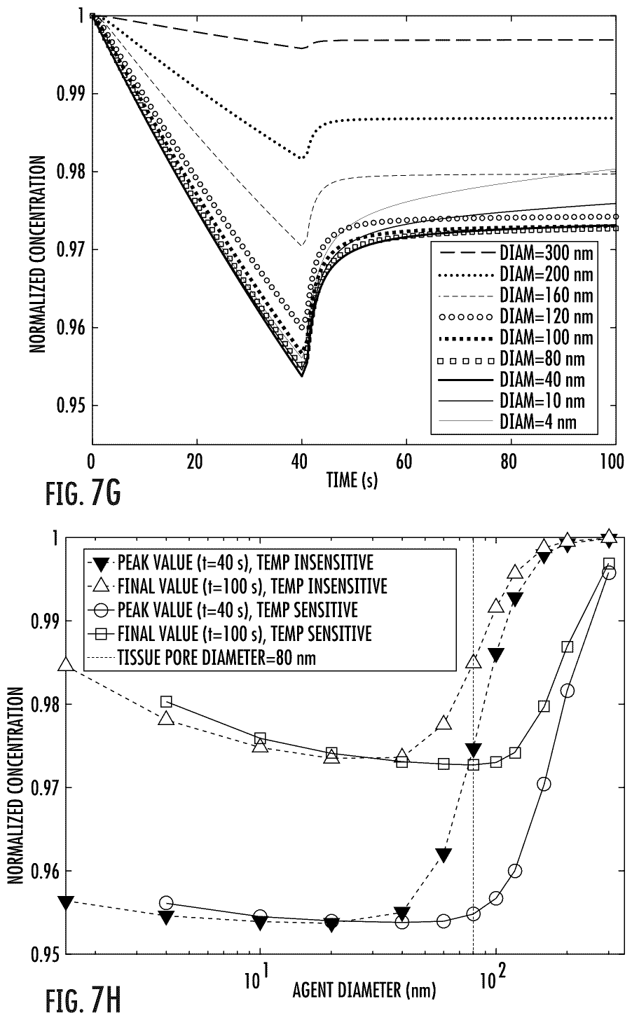

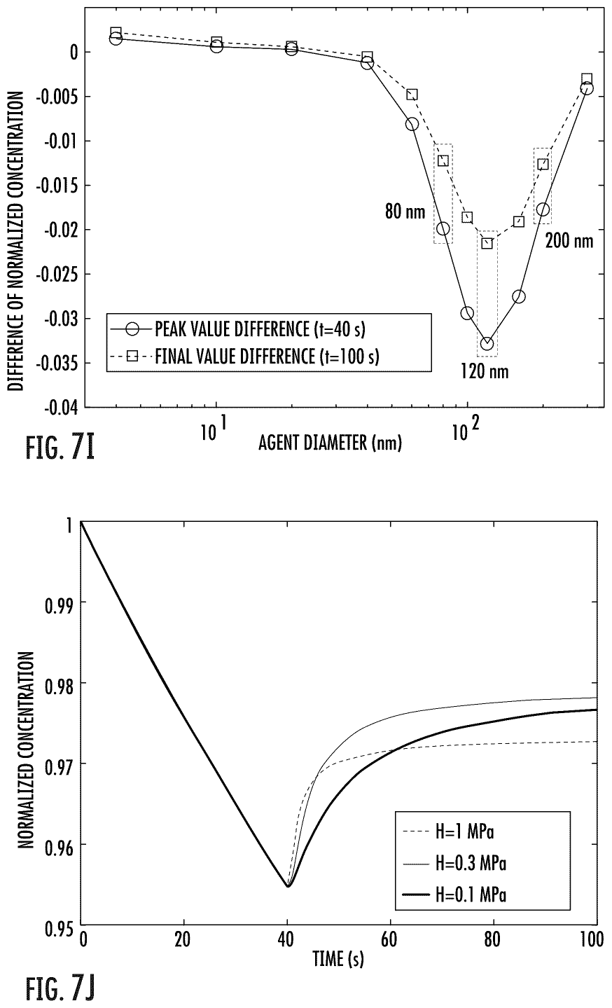

Similar to FIG. 5, FIG. 7 shows the corresponding results for temperature-sensitive agents. FIG. 7A-B show the 2D distribution of the normalized concentration on the xy planes (z=0) and xz plane (y=0), respectively, directly after ultrasound is off (t=t₀=40 s). FIG. 7C-D display similar results when t=100 s. FIG. 7E-F show the 1D distribution of normalized concentration along the x and z directions at different times, respectively. The major difference is that the average diameter of the agent in FIG. 7A-F is 80 nm, whereas it is 40 nm in FIG. 5A-F. Comparing FIG. 7A-F with FIG. 5A-F, concentration changes are evidently similar in the two situations. Therefore, using a temperature-sensitive agent allows using an agent with a larger size to achieve similar performance in concentration change compared with using a temperature-insensitive agent. This is beneficial for improving delivery efficiency because a larger-sized agent will have a bigger volume and payload capacity, such as for drugs, proteins, and other molecules.

FIG. 7G plots the dynamic variation of the normalized concentration at the focal center as a function of time for agents with different diameters. When comparing FIG. 7G with FIG. 5G, for the same agent diameter, the concentration change is higher for temperature-sensitive agents than for temperature-insensitive agents. For further quantitative comparison of the concentrations in FIG. 7G and FIG. 5G, the normalized concentrations were selected at two time points, t=40 and 100 s, and they were plotted as a function of the agent diameter in FIG. 7H. The curve for the temperature-sensitive agents acquired at t=40 s (the line with open circles) is shifted toward the right-hand side compared with that for the temperature-insensitive agents (the line with filled inverted triangles). Similar results can also be found for data acquired at t=100 s by comparing the line with squares with the line with open triangles. The vertical dotted line indicates that the tissue pore size is 80 nm. As discussed above, preferred agent size for temperature-insensitive agents can be between 10 and 40 nm for balancing transportability and diffusivity. Notably, the preferred range of agent size for temperature-sensitive agents is widely broadened and can reach a range between 10 and 120 nm. It is important to note that, in some embodiments, when a USF agent is shrunk in a high-temperature environment, it can expel water molecules out of the agent instead of expelling the payloads. This feature provides an opportunity to allow transporting large-sized agents, such as those measuring 40-120 nm, in tissue using this SIF-TUM technology combined with temperature-sensitive agents. Otherwise, it would be extremely difficult to transport such big agents. FIG. 7I further plots the difference of the normalized concentration between the temperature-sensitive and -insensitive agents as a function of agent diameter. When the diameter is ≤40 nm or ≥300 nm, both types of agents have similar concentration changes.

This can be understood as follows. When the agent has a size either much smaller or much bigger than the tissue pore size, the transportability of the agent is much less dependent on the size. However, when the agent has a size close to or slightly above the pore size, such as 80-200 nm in this example, the feature of thermally reducing size can help improve transportability. This can be seen from FIG. 7I, in which the difference of the concentration change between the two types of agents is shown for agents with a size between 80 and 200 nm. This result indicates that if the agent size is within the range of 80-200 nm, using a temperature-sensitive agent should have a higher transportability than using a temperature-insensitive agent. However, when temperature-sensitive agents have been selected using a size within the preferred range of 10-120 nm, it is a better choice compared with using an agent with a size >120 nm, because the absolute value of the concentration change is higher.

FIG. 7J shows the effect of the apparent module on the tissue recovery rate. The same result as FIG. 5J can be drawn that the higher H will lead to a faster initial recovery rate. In addition, FIG. 7J shows a higher concentration change than FIG. 5J. This further indicates that a temperature-sensitive agent has a higher transportability than a temperature-insensitive agent if the agent diameter is appropriate, as described herein.

### Selection of Parameters

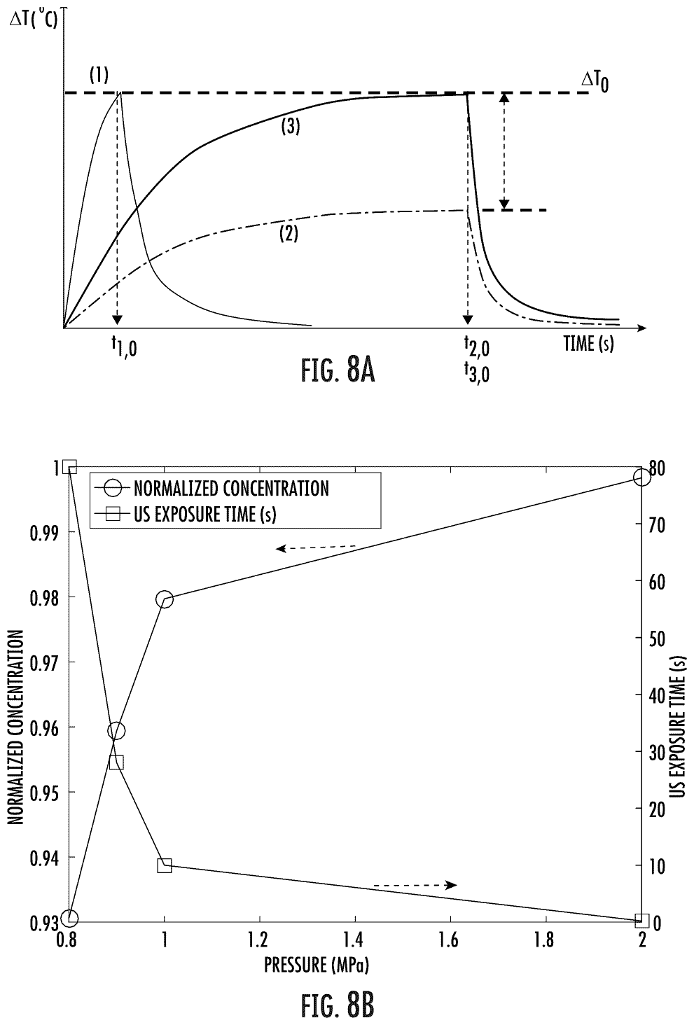

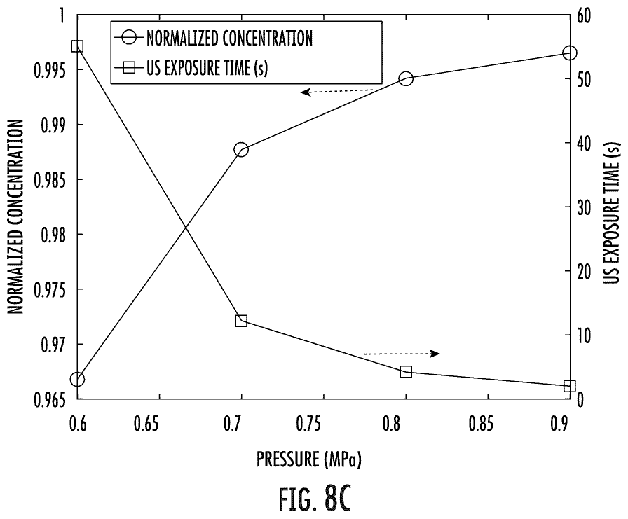

In practice, to achieve preferred or other selected transport efficiency, it is possible to adjust the ultrasound pressure and exposure time. For example, to maintain the spatial peak temperature ΔT₀ at a fixed value, the strength of the ultrasound pressure, described by |P₁| as a spatial function and further controlled by the spatial peak pressure amplitude by Pₐ in this Example, and the exposure time t₀ can be selected to achieve the highest (or some other preferred or desired) agent concentration change. In general, increasing the pressure strength can be paired with reducing the exposure time or vice versa. Similarly, in some embodiments, reducing Pₐ and increasing t₀ can enable an increase the agent concentration change. This is schematically illustrated in FIG. 8A. First, in some example embodiment, the ultrasound pressure (Pₐ) should not be too strong because a strong pressure will quickly increase ΔT to reach the upper limit of ΔT₀ in a short time of t_{1,0}, which will sacrifice the amount of the concentration change (see situation (1) in FIG. 8A; the thin line). Second, the strength is not so weak that ΔT will reach a stable final value that can never reach the upper limit of ΔT₀ because of the existence of thermal diffusion, which will waste the allowed space of ΔT₀ and provide a low amount of the concentration change (see situation (2) in FIG. 8A; the dot-dashed line). Third, the strength is a value that can increase the tissue temperature close to or equal to ΔT₀ (see situation (3) in FIG. 8A; the bold line). Thus, the exposure time t₀ can be extended for improving or otherwise modifying the amount of the concentration change compared with situations (1) and (2). For example, maintaining ΔT₀ at 4.8° C., FIG. 8B shows the simulated data of the normalized concentration of the agent (C/C₀) at the center of the ultrasound focus as a function of the spatial peak pressure (Pₐ) at a different exposure time of to. By reducing Pₐ from 2 to 0.8 MPa, the exposure time t₀ can be extended from 0.2 to 80 s (see the line with squares in FIG. 8B). Accordingly, the agent concentration change (ΔC/C₀=(C₀−C)/C₀) at the center of the ultrasound focus can increase from 0.17% to 7% (see the line with circles in FIG. 8B). Further, significantly reducing the pressure well below 0.8 MPa can continuously increase the exposure time (to), but the temperate rise will be difficult to reach the preset value of 4.8° C. because of the existence of thermal diffusion. In some preferred embodiments, instead of only using temperature, a thermal dose considering both temperature and time, such as 4.3° C. with equivalent minutes, may be used for thermal safety. Therefore, the eventual upper limit of the exposure time (to) can be constricted by the required thermal dose.

### How R_{f} was Calculated Based on the Size and Distribution of the Agents and Tissue Pore Size

Usually, biological soft tissues can be approximated as a porous media. The retardation factor (R_{f}) was calculated based on the distributions of tissue pore size and agent size following the algorithm described below. Each possible agent diameter (Dᵢ) was compared with all the possible pore diameters (Dⱼ). If Dᵢ<Dⱼ, the possibility of the particle with the diameter of Dᵢ passing through the pore with the diameter of Dⱼ is one (i.e., pᵢⱼ=1). Otherwise, when Dᵢ≥Dⱼ, pᵢⱼ is 0. Thus, by varying the indexes of i and j through all their possible values, a 2D matrix of pᵢⱼ can be established. Then, pᵢⱼ was summed along the j direction, which gives pᵢ representing the possibility of the particle with the diameter of Dᵢ passing through the tissue. If pᵢ was summed further along all possible i, a single number of p₀ can be found, which represents the possibility of the entire agent passing through the tissue. To normalize this possibility p₀, a unit matrix was formed by allowing all the element of pᵢⱼ equal to 1 and summing all of them together to obtain a single value of pₘ. This value represents the maximum possibility for the scenario when all the agent particles are smaller than the smallest pore size so that they can freely move through the pores in the tissue by following the flow of the interstitial fluid. Thus, the value of p₀/pₘ will be the normalized possibility of the agent passing through the tissue. Physically, if p₀/pₘ is equal to 1, all the agent particles are so small compared with the tissue pore size that they can freely move in the tissue by following the fluid. However, if p₀/pₘ is equal to 0, the particles are so large that no particles can pass through tissue pores, and thus, they cannot be transported via the flow. When the distribution of the agent size is partially overlapped with that of the tissue pore size, the value of p₀/pₘ will fall between 0 and 1. However, the Gaussian distribution assumptions are just for the convenience of calculations, and any other distributions can also be used without losing the generality of the models. In addition, other agent factors, such as surface charges and shapes, may also affect agent transport.

### F. Conclusion

In this Example, a focused ultrasound beam was modeled to induce interstitial fluid streaming in deep tissue via momentum transfer between the ultrasound wave and tissue interstitial fluid. Biological tissue is considered a bi-phase medium including both a fluid and a solid matrix. The interaction between the ultrasound and the two components (i.e., fluid and solid) was modeled with equations and demonstrated via numerical simulations. Both mechanical and thermal effects induced by the ultrasound have been considered in the models.

The results show that a macroscopic fluid streaming can be induced with a peak velocity at the level of microns/second in a millimeter focal zone. Not intending to be bound by theory, it is believed that the streaming mechanism is as follows. The ultrasound wave transfers its momentum to tissue fluid, which leads to a “splashing” of the fluid out of the focal volume. Because of the finite value of tissue hydraulic conductivity, the “splashed” fluid further leads to the elevation of the hydrostatic pressure in the focal volume. This elevated hydrostatic pressure can generate a macroscopic exudation flow. The entire procedure can be visualized as a scenario in which the ultrasound focus squeezes the tissue in the focal volume along all three dimensions (x, y, and z). The velocity of this exudation flow is found to be determined by a term of [∇P₁·∇P₁*], which is the dot product between the gradient of the 1st-order ultrasound pressure wave and its complex conjugate, rather than the ultrasound pressure itself. Furthermore, after ultrasound is turned off, the fluid can backflow into the focal volume because of the tissue elasticity of the solid matrix. The speed of the recovery of the fluid (or solid matrix) right after the ultrasound is off is usually high because of the high apparent modulus. This speed decays quickly as time goes on. Thus, it takes a much longer time for the rest of the fluid to flow back.

This method provides a unique opportunity to control the fluid and the agents that are dissolved in the fluid. SIF-TUM can be used to externally accelerate agent transport in deep tissue and further control the agent distribution, including by using method steps and other parameter as described herein. SIF-TUM will be useful for critical applications such as the efficient delivery of therapeutic and theranostic agents.

### G. References

Aglyamov S, Karpiouk A B, Linskii Y, Zabolotskaya E and Emelianov S 2007 Motion of a solid sphere in a viscoelastic medium in response to applied acoustic radiation force: Theoretical analysis and experimental verification The Journal of the Acoustical Society of America 122 1927-36
Anand A and Kaczkowski P J 2008 Noninvasive measurement of local thermal diffusivity using backscattered ultrasound and focused ultrasound heating Ultrasound in medicine & biology 34 1449-64
Baxter L T and Jain R K 1989 Transport of fluid and macromolecules in tumors. I. Role of interstitial pressure and convection Microvasc Res 37 77-104
Boucher Y, Brekken C, Netti P A, Baxter L T and Jain R K 1998 Intratumoral infusion of fluid: estimation of hydraulic conductivity and implications for the delivery of therapeutic agents British Journal of Cancer 78 1442-8
Bruus H 2011 Acoustofluidics 1: Governing equations in microfluidics Lab on a Chip 11 3742-51
Bruus H 2012a Acoustofluidics 2: Perturbation theory and ultrasound resonance modes Lab on a Chip 12 20-8
Bruus H 2012b Acoustofluidics 7: The acoustic radiation force on small particles Lab on a Chip 12 1014-21
Cheng B B, Bandi V, Wei M Y, Pei Y B, D'Souza F, Nguyen K T, Hong Y and Yuan B H 2016 High-Resolution Ultrasound-Switchable Fluorescence Imaging in Centimeter-Deep Tissue Phantoms with High Signal-To-Noise Ratio and High Sensitivity via Novel Contrast Agents Plos One 11
de Smet M, Heijman E, Langereis S, Hijnen N M and Grüll H 2011 Magnetic resonance imaging of high intensity focused ultrasound mediated drug delivery from temperature-sensitive liposomes: An in vivo proof-of-concept study Journal of Controlled Release 150 102-10
Deckers R and Moonen C T W 2010 Ultrasound triggered, image guided, local drug delivery Journal of Controlled Release 148 25-33
Doherty J R, Trahey G E, Nightingale K R and Palmeri M L 2013 Acoustic radiation force elasticity imaging in diagnostic ultrasound IEEE Transactions on Ultrasonics, Ferroelectrics, and Frequency Control 60 685-701
Dromi S, Frenkel V, Luk A, Traughber B, Angstadt M, Bur M, Poff J, Xie J, Libutti S K, Li K C P and Wood B J 2007 Pulsed-High Intensity Focused Ultrasound and Low Temperature-Sensitive Liposomes for Enhanced Targeted Drug Delivery and Antitumor Effect Clin Cancer Res 13 2722
Duhr S and Braun D 2006 Why molecules move along a temperature gradient Proceedings of the National Academy of Sciences 103 19678-82
Ehlers W and Markert B 2001 A Linear Viscoelastic Biphasic Model for Soft Tissues Based on the Theory of Porous Media Journal of Biomechanical Engineering 123 418-24
El Ghamrawy A, de Comtes F, Koruk H, Mohammed A, Jones J R and Choi J J 2019 Acoustic Streaming in a Soft Tissue Microenvironment Ultrasound in Medicine & Biology 45 208-17 Frenkel V 2008 Ultrasound mediated delivery of drugs and genes to solid tumors Adv Drug Deliver Rev 60 1193-208
Grüll H and Langereis S 2012 Hyperthermia-triggered drug delivery from temperature-sensitive liposomes using MRI-guided high intensity focused ultrasound Journal of Controlled Release 161 317-27
Hancock H, Smith L, Cuesta J, Durrani A, Angstadt M, Palmeri M, Kimmel E and Frenkel V 2009 Investigations into Pulsed High-Intensity Focused Ultrasound-Enhanced Delivery: Preliminary Evidence for a Novel Mechanism Ultrasound in Medicine & Biology 35 1722-36 Jain R K 1999 Transport of Molecules, Particles, and Cells in Solid Tumors Annu Rev Biomed Eng 1 241-63
Kwan M K, Michael Lai W and Van Mow C 1984 Fundamentals of fluid transport through cartilage in compression Annals of Biomedical Engineering 12 537-58
Lee E S, Lee J Y, Kim H, Choi Y, Park J, Han J K and Choi B I 2013 Pulsed high-intensity focused ultrasound enhances apoptosis of pancreatic cancer xenograft with gemcitabine Ultrasound in Medicine & Biology 39 1991-2000
Liu L J and Schlesinger M 2015 Interstitial hydraulic conductivity and interstitial fluid pressure for avascular or poorly vascularized tumors Journal of Theoretical Biology 380 1-8
Liu Y, Yao T, Cai W, Yu S, Yi H, Nguyen K and Yuan B 2020 A Biocompatible and Near-Infrared Liposome for In Vivo Ultrasound-Switchable Fluorescence Imaging Advanced Healthcare Materials 9 1901457
O'Neill B E, Vo H, Angstadt M, Li K, Quinn T and Frenkel V 2009 Pulsed High Intensity Focused Ultrasound Mediated Nanoparticle Delivery: Mechanisms and Efficacy in Murine Muscle Ultrasound in Medicine & Biology 35 416-24
Park S M, Kim M S, Park S-J, Park E S, Choi K-S, Kim Y-s and Kim H R 2013 Novel temperature-triggered liposome with high stability: Formulation, in vitro evaluation, and in vivo study combined with high-intensity focused ultrasound (HIFU) Journal of Controlled Release 170 373-9
Pei Y B, Wei M Y, Cheng B B, Liu Y, Xie Z W, Nguyen K and Yuan B H 2014 High resolution imaging beyond the acoustic diffraction limit in deep tissue via ultrasound-switchable NIR fluorescence Sci Rep-Uk 4 4690
Pitt W G 2008 Ultrasound in Drug and Gene Delivery Adv Drug Deliver Rev 60 1095-6 Prieur F and Sapozhnikov O A 2017 Modeling of the acoustic radiation force in elastography The Journal of the Acoustical Society of America 142 947-61
Raghavan R 2018 Theory for acoustic streaming in soft porous matter and its applications to ultrasound-enhanced convective delivery Journal of therapeutic ultrasound 6 6
Ramanujan S, Pluen A, McKee T D, Brown E B, Boucher Y and Jain R K 2002 Diffusion and Convection in Collagen Gels: Implications for Transport in the Tumor Interstitium Biophysical Journal 83 1650-60
Ranjan A, Jacobs G C, Woods D L, Negussie A H, Partanen A, Yarmolenko P S, Gacchina C E, Sharma K V, Frenkel V, Wood B J and Dreher M R 2012 Image-guided drug delivery with magnetic resonance guided high intensity focused ultrasound and temperature sensitive liposomes in a rabbit Vx2 tumor model Journal of Controlled Release 158 487-94
Sapozhnikov O A 2012 An exact solution to the Helmholtz equation for a quasi-Gaussian beam in the form of a superposition of two sources and sinks with complex coordinates Acoustical Physics 58 41-7
Sarvazyan A, Rudenko O and Nyborg W 2010 Biomedical Applications of Radiation Force of Ultrasound: Historical Roots and Physical Basis Ultrasound in Medicine & Biology 36 1379-94
Sarvazyan A, Rudenko O, Swanson S, Fowlkes J B and Emelianov S Y 1998 Shear wave elasticity imaging: a new ultrasonic technology of medical diagnostics Ultrasound in Medicine & Biology 24 1419-35
Siggers J H, Leungchavaphongse K, Ho C H and Repetto R 2014 Mathematical model of blood and interstitial flow and lymph production in the liver Biomechanics and Modeling in Mechanobiology 13 363-78
Swartz M A and Fleury M E 2007 Interstitial Flow and Its Effects in Soft Tissues Annu Rev Biomed Eng 9 229-56
Tebebi P A, Kim S J, Williams R A, Milo B, Frenkel V, Burks S R and Frank J A 2017 Improving the therapeutic efficacy of mesenchymal stromal cells to restore perfusion in critical limb ischemia through pulsed focused ultrasound Sci Rep-Uk 7 41550
Vyas R and Rustgi M L 1992 Green's function solution to the tissue bioheat equation Medical Physics 19 1319-24
Wongsuwarn S, Vigolo D, Cerbino R, Howe A M, Vailati A, Piazza R and Cicuta P 2012 Giant thermophoresis of poly(N-isopropylacrylamide) microgel particles Soft Matter 8 5857-63
Wu J 2018 Acoustic Streaming and Its Applications Fluids 3 108
Yao T, Liu Y, Ren L and Yuan B 2021 Improving sensitivity and imaging depth of ultrasound-switchable fluorescence via an EMCCD-gain-controlled system and a liposome-based contrast agent Quantitative imaging in medicine and surgery 11 957-68
Yao T, Yu S, Liu Y and Yuan B 2019a In vivo ultrasound-switchable fluorescence imaging Sci Rep 9 9855
Yao T, Yu S, Liu Y and Yuan B 2019b In vivo ultrasound-switchable fluorescence imaging Sci Rep-Uk 9 9855
Yao W, Shen Z and Ding G 2013 Simulation of Interstitial Fluid Flow in Ligaments: Comparison among Stokes, Brinkman and Darcy Models International Journal of Biological Sciences 9 1050-6
Yeh H-L and Juárez J J 2021 Ultrasound-enhanced diffusion and streaming of colloids in porous media Experimental Thermal and Fluid Science 121 110282
Yu S, Wang Z, Yao T and Yuan B 2020a Near-infrared temperature-switchable fluorescence nanoparticles Quantitative imaging in medicine and surgery 11 1010-22
Yu S, Yao T, Liu Y and Yuan B 2020b In vivo ultrasound-switchable fluorescence imaging using a camera-based system Biomed. Opt. Express 11 1517-38
Yu S, Yao T, Liu Y and Yuan B 2020c In vivo ultrasound-switchable fluorescence imaging using a camera-based system Biomed Opt Express 11 1517-38
Yuan B, Uchiyama S, Liu Y, K. N T and Alexandrakis G 2012 High-resolution imaging in a deep turbid medium based on an ultrasound-switchable fluorescence technique Appl Phys Lett 101 033703
Yuh E L, Shulman S G, Mehta S A, Xie J, Chen L, Frenkel V, Bednarski M D and Li K C P 2005 Delivery of Systemic Chemotherapeutic Agent to Tumors by Using Focused Ultrasound: Study in a Murine Model Radiology 234 431-7
Zhang Y, Czerwonka L, Tao C and Jiang J J 2008 A biphasic theory for the viscoelastic behaviors of vocal fold lamina propria in stress relaxation The Journal of the Acoustical Society of America 123 1627-36
Ziadloo A, Xie J and Frenkel V 2013 Pulsed focused ultrasound exposures enhance locally administered gene therapy in a murine solid tumor model The Journal of the Acoustical Society of America 133 1827-34

### EXAMPLE 2

### Monitoring of the Distribution of Agents Using SIF-TUM

### A. General

It is highly desirable to develop a non-invasive technology to externally control the agent distribution in a tumor to improve the treatment or diagnosis efficiency. To date, the transport and distribution of therapeutic or diagnostic agents in the interstitial space of a tumor are uncontrollable and mainly dependent on natural and random motions. The two natural transport mechanisms are diffusion due to concentration gradient and convection via natural interstitial fluid flow, which are further dependent on a tissue's microenvironments. While diffusion is a very slow process (especially for large sized agents, such as large molecules, proteins, nanoparticles, etc.), the elevated interstitial fluid pressure (IFP) in a tumor significantly reduces the convection transport efficiency. This leads to a severe problem that the distribution of therapeutic or diagnostic agents is heterogeneous.

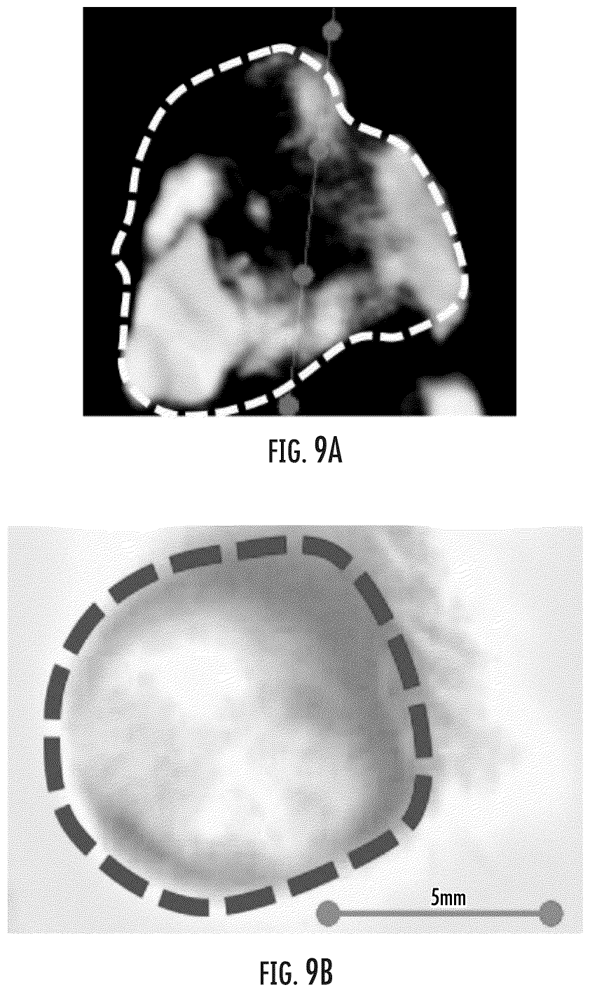

FIG. 9 shows two examples in which imaging agents accumulated more in the peripheral areas than in central areas because of elevated IFP and/or central necrosis. FIG. 9A shows a micro-CT image of the non-uniform distribution of nanoparticles in spontaneous live breast tumor tissue outlined in dashes in a live mouse, and FIG. 9B displays a fluorescence image of the non-uniform distribution of nanoparticles in a pancreatic tumor outlined in dashes in a live mouse. This is commonly seen in tumor chemotherapy. Thus, an insufficient drug dosage can occur at these areas, which can lead to a high risk of tumor regrowth and metastases. Similar issues also occur in other methods when agents are locally injected into a tumor. For example, it is common to intratumorally or intramuscularly inject various agents, such as cells, antibodies, proteins, gene- or virus-encapsulated nanoparticles or imaging contrast agents, for different treatment and diagnosis purposes in immunotherapy, viral therapy, photodynamic therapy, photo-thermal therapy, etc. After injections, the agent distribution is critical. Preferably they should cover the target areas. However, without a tool to control agent distribution, distributions mainly rely on natural and random motions.

To be able to control agent distribution, the external driving force should preferably be strong enough to overcome the natural recovery forces in the tumor, usually caused by agent diffusion and natural fluid convection, and the external driving force should preferably be controllable in strength, space, and time to accurately control agent motion in a tumor. After re-distribution, agents should preferably stay at the desired locations for a long enough time to avoid undesired recovery caused by the natural recovering forces.

As compared to uncontrolled enhancement of agent distribution, SIF-TUM provides control of agent distribution. Controlling agent distribution is more challenging compared with the uncontrolled enhancement of agent delivery, and it is also more desirable. SIF-TUM gently “squeezes” and “relaxes” tissue to generate fluid flows. In uncontrolled enhancement, it is possible to physically “open” cell-cell junctions or “expand” tissue's space, and ultrasound pressure itself can be used to provide the desired effect. In controlled SIF-TUM, the spatial gradient of ultrasound pressure, instead of pressure itself, can be used to direct agent transport. Increasing the pressure gradient is preferred in some embodiments of SIF-TUM, rather than merely increasing the pressure itself. Additionally, in some uncontrolled enhancement methods, to avoid a large temperature rise, the duty cycle of the ultrasound exposure is limited no more than 1-5%. In some embodiments of controlled SIF-TUM, a low pressure with a high gradient and a large duty cycle (100%) can provide a much higher transport efficiency. Further, after ultrasound is turned off, agents can also naturally backflow into their original locations. This backflow can make the change in concentration not observable or inefficient. However, this backflow can be reduced, minimized, or avoided using SIF-TUM.

The Example herein presents results for the monitoring of the distribution of agents, the ability to assess the re-distribution of agent, and the feedback and guidance for the control of agents using SIF-TUM. This work is helpful for cancer therapeutic and diagnostic efficacy and efficiency by externally controlling agent distribution in tumors.

### B. Introduction

### Process of SIF-TUM Method

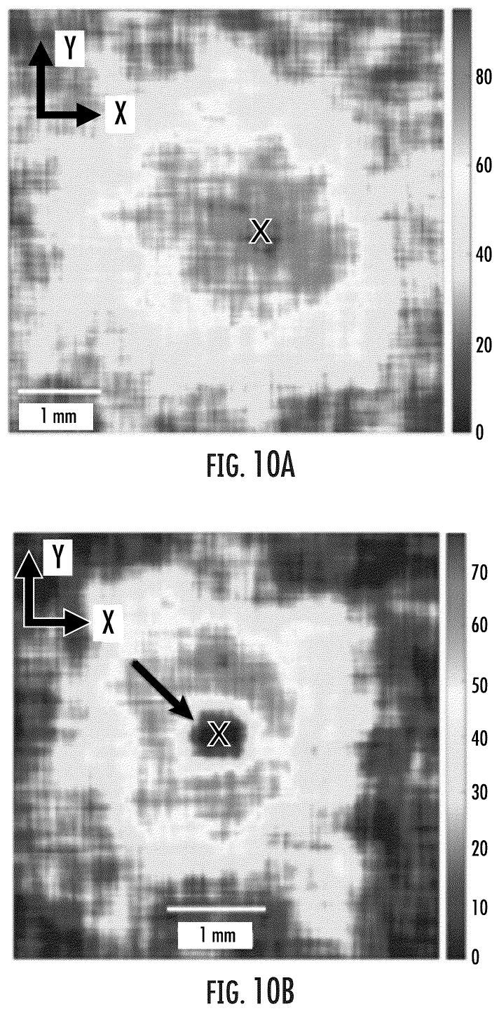

SIF-TUM technology gently “squeezes” the tissue in a small ultrasound focal volume (˜0.7 mm³) from all the directions. For example, FIG. 10A-B respectively show a normal and an abnormal ultrasound fluorescence USF signal from a BXPC3 pancreatic tumor. Clearly, in FIG. 10B, the USF signal is lost at the ultrasound focal area, indicated by the cross at the center of the image. The loss of USF signal is because the USF contrast agents (i.e. nanoparticles of ˜65 nm) can be “pushed” out of the focal volume under using a highly focused ultrasound beam. SIF-TUM can be imagined as a scenario of “3-dimensionally compressing and relaxing an internal small portion of a water-filled sponge”. The squeezing and relaxation can be modeled into equations that are listed in Table 1 in Example 1.

The “squeezing” of tissue's interstitial fluid out of the focal volume occurs via the transfer of ultrasound momentum. FIG. 2A shows the 2D distribution of the amplitude of the 1^{st}-order ultrasound pressure wave (|P̅₁|) on the xz plane with a peak value of 1 MPa. P is the complex amplitude of the ultrasound oscillation wave. In this Example, the ultrasound beam has the following parameters. The frequency is 2.5 MHz. The full-width-at-half-maximum (FWHM) of the focus along the lateral (x and y) and axial (z) direction is σₓ=σ_{y}=0.5 and σ_{z}=3.5 mm, respectively. Tissue absorbs a portion of the momentum and the energy of the ultrasound wave in its focal volume (box in FIG. 2A). The absorption of the energy will lead to tissue temperature increase, which will be discussed in next sections. The absorption of the momentum will lead to the interstitial fluid being “splashed” away from the focal volume (outwards, FIGS. 2C and D) and the solid matrix being volumetrically compressed (inwards, data not shown). This is also called momentum transfer from the ultrasound wave to the tissue. This can be described as an ultrasound-induced elevation of the hydrostatic pressure (P₂) of the interstitial fluid in the focal volume (FIG. 2B in which the peak value of P₂ reaches ˜14,000 Pₐ in this Example).

Based on Darcy's law, the elevated fluid pressure (P₂) will generate an exudation flow (W₂, FIG. 2C-D). Thus, a small amount of tissue's interstitial fluid will be “squeezed” out of the focal volume. The flow (W₂) will form the SIF-TUM convection transport mechanism to move the agents out of the focal volume (FIG. 2C-D). Because the exudation velocity, W₂ is a vector. FIG. 2C shows its x and y components on the xy plane. FIG. 2D shows its x and z components on xz plane. Each arrow points the flow direction, and its length is proportional to the flow speed. Clearly, during the period of ultrasound is applied, the fluid flows out of the focal volume along all the three directions (x, y, and z). FIG. 2E-F display the magnitude of W₂ (i.e. |W₂|) on the xy and xz plane, respectively. The velocity can reach as high as 0.25 microns/s in this example at the positions surrounding the center point (but not the center point), where the gradients of P̅₁ and P₂ (i.e., ∇P̅₁ and ∇P₂) achieve the maxima.

P₂ is diffused in space because it is governed by a Poisson-like equation (i.e., equation 1 in Table 1). Thus, the distribution of P₂ will be spatially expanded compared with that of |P̅₁|. This can be seen by comparing the lateral and axial FWHMs of P₂ in FIG. 2B (3.6 and 10.5 mm, respectively) with those of |P̅₁| in FIG. 2A (0.5 and 3.5 mm, respectively). This diffusion effect of P₂ will be favorable for the purpose of controlling agent distribution. This is because the agent distribution is controlled by P₂-induced flow, and therefore, a larger space of P₂ (compared with the space of |P̅₁|) will more efficiently affect agent distribution.

The Interstitial Fluid of Tissue Flows Back when Ultrasound is Off

After ultrasound is turned off, a portion of the “pumped-out” fluid will quickly flow back into the original focal volume. The rest of the fluid will flow back very slowly. Thus, it provides an opportunity for a portion of the fluid/agent to remain outside of the focal volume for a time period long enough to be further transported by another ultrasound exposure. The mechanical index (MI) of the ultrasound exposure can be maintained below the FDA-required safety threshold of MI<1.9, and the thermal indexes (TI) below the AIUM-required safety threshold TI<6 (American Institute of Ultrasound in Medicine). Thus, this procedure can be safely repeated many times. Therefore, the total amount of agents that are “pumped” out can be accumulated by controlling the ultrasound exposure time at each location.

Because the size of the ultrasound focus is small, the spatial distribution of agents can be controlled by 3D scanning the ultrasound focus point-by-point though the interested volume. FIG. 3A shows the fluid is flow back into the focal volume at 1 s after the ultrasound is turned off. FIG. 3B-C show the corresponding spatial distribution of the magnitude of the backflow velocity on xy and xz plane, respectively. FIG. 3D displays the reduction of the maximum backflow velocity as a function of time after the ultrasound is turned off. Clearly, the initial maximum velocity can reach as high as 1.8 microns/s and quickly reduces to 0.06 microns/s at 8.4 s. Then, the velocity decays very slowly and remains a small value for a long time.

### Agent Accumulation in the Regions Outside the Focal Volume

The ultrasound-induced flow W₂ describes the motion of the interstitial fluid in tissue. Agents are typically considered solutes that are dissolved in the fluid. Thus, there is a relationship between the motion of the agents (i.e., solutes) and the motion of the fluid. A retardation factor, R_{f}, has been defined as the ratio of the agent velocity to the interstitial fluid velocity. It is reasonable to have the following assumptions. When an agent is small enough, such as <1-2 nm, R_{f} is equal or close to 1. Thus, the agent will move with a velocity that is the same as or similar to the fluid velocity W₂. When the size of the agent is too large to be able to pass through the pore of tissue's extracellular matrix (ECM), R_{f} is equal or close to zero. This means the agent will not be able to move, even though the fluid is moving with a velocity of W₂. The following two situations are examples and describe the agent accumulation in the regions outside the ultrasound focal volume.

### Temperature-Insensitive Agents

The feature of the agents in this category is that these agents are not sensitive to temperature. Thus, their physical sizes and other properties, such as fluorescence, do not change or do not change substantially as a function of the temperature, at least in the temperature range from tissue background temperature to the peak temperature increased by the ultrasound. Therefore, their motions are determined by the flow of the interstitial fluid induced by ultrasound. The relationship between the velocity of the agents and that of the fluid is quantified by the retardation factor R_{f}, a size-dependent factor. However, because they are temperature insensitive, R_{f} will be independent of the temperature and remain the same during the periods of ultrasound is on and off. In this category, it may include agents such as small and large molecules, nanoparticles, liposomes, micelles, quantum-dots, polymers, proteins, DNAs, antibodies, genes/virus and various carriers, etc. As mentioned above, the agent may move with a velocity that is the same as or similar to or smaller than the fluid velocity W₂. For this situation, during the period of ultrasound is on, the agents will be pushed out of the focal volume by following the fluid flow. On the other hand, a portion of the agents will also flow back into the original volume quickly after turning off the ultrasound by following the fast backflow of the fluid. The rest of “pumped-out” agents will very slowly flow back by following the slow backflow of the fluid as discussed above. Thus, it provides an opportunity for a portion of the fluid and the agent to remain outside the focal volume for a time period long enough to be further transported by another ultrasound exposure. This uses the slow relaxation speed (or recovery speed) of soft tissue after being compressed. The initial fast relaxation speed (or recovery speed) will bring a portion of agents back into the original volume and reduce the transport efficiency. However, it can also help to avoid tissue being overcompressed and lacking fluid. Therefore, if needed, this procedure can be safely repeated so that the total amount of agents that are “pumped” out can be accumulated by controlling the ultrasound exposure time at each location. The simulated results for this situation can be found in FIG. 5.

### Extremely Temperature-Sensitive Agents

Agents that are extremely sensitive to temperature experience significant and/or sharp changes in their physical sizes and other properties, such as fluorescence signal if available, as a function of the temperature in the temperature range from tissue background temperature to the peak temperature increased by the ultrasound. Therefore, the retardation factor R_{f}, a size-dependent factor, will be dependent on the temperature and will be different during the periods of ultrasound is on and off. USF contrast agents belong to this type. For each USF agent, usually a temperature threshold (T_{L}) exists and is slightly above the body temperature of 37° C. (i.e. T_{L}>37° C. such as ˜38-39° C.) for in vivo uses. When the tissue in the ultrasound focal volume is heated by the ultrasound above this threshold, the size of the nanoparticles will be quickly (milliseconds) and dramatically reduced. On the other hand, when temperature is cooled down below the threshold, the size of nanoparticles will quickly recover back to the original size. Temperature-induced size changes provide an opportunity for accumulating agents out of the ultrasound focal volume and improving the efficiency of the ultrasound-induced agent transport.

Before applying the ultrasound, the nanoparticle or agent has an initial averaged size L₀, which corresponds to a retardation factor R_{f0}. When ultrasound is applied, the tissue temperature in the focal volume is increased above the temperature threshold of the agent (FIG. 4, i.e. T_{BG}+ΔT>T_{L}, where T_{BG} is tissue background temperature, ΔT is the ultrasound-induced temperature increase, and T_{L} is the temperature threshold of the agent). Thus, the agent in this region will shrink, and the averaged size is reduced to L₁, which corresponds to a larger retardation factor (R_{f}). Because the size is reduced (i.e. L₁<L₀), it can be expected that the retardation factor is significantly increased (i.e. R_{f1}>R_{f0}). Physically, this means that the agent will be easier to move by following the fluid flow, since the size is reduced. Thus, during the ultrasound exposure period, the agent will move out of the focal volume relatively easily. The temperature outside the focal volume is equal to the tissue background temperature, which is lower than the temperature threshold of the agent (i.e. T_{BG}≈37° C.<T_{L}). Thus, after the agents move out of the focal volume, the agents will recover their size, back to the original size of L₀, and the retardation factor back to R_{f0}. Physically, this means that once the agents are “pumped” out of the focal volume (i.e., out of the hot area), they will be difficult to flow back after ultrasound is off because their sizes are increased and their mobility with the flow is reduced because of the lower environmental temperature.

In some such embodiments, when ultrasound is turned off, the speed of the temperature recovery back to the background temperature is much faster than the backflow velocity of the fluid. In this case, the temperature is already back to background temperature before the fluid is back. Thus, the “pumped” out agents will remain their original large size L₀, even though a small portion of them can flow back. With the original size L₀, their mobility is weaker compared with that with the shrunken size. Accordingly, it can minimize the backflow of agents into the original focal volume. This situation occurs for most biological soft tissues. Usually, the temperature diffusion speed is higher than the backflow velocity of the interstitial fluid. Both phenomena are described by diffusion equations described by the equations 2b and 3b in Table 1. Therefore, both velocities are determined by their diffusion coefficients (i.e. KH in equation 2b and kₜ/ρ₀cₜ in equation 3b). Based on literature data, the thermal diffusion coefficient kₜ/ρ₀cₜ is ˜1.34×10⁻⁷ (m²/s), and KH is 4×10⁻⁸ (m²/s) (with K=4×10⁻¹⁴ m⁴/N/S and H=1 MPa). The simulated results for this situation can be found in FIG. 7.

### Other Driving Forces

In addition to the ultrasound-induced flows of interstitial fluid of SIF-TUM (CR_{f}W₂), two other mechanisms that can also drive agent re-distribution are diffusion due to the concentration gradient (−D_{BD}∇C) and thermophoresis due to the ultrasound-induced temperature gradient (−CD_{TP}∇(ΔT)). Equation 4 listed in Table 1 quantitatively describes these three mechanisms. C is the agent concentration, and ΔT is the ultrasound-induced temperature change. D_{BD} and D_{TP} are agent diffusion coefficient and thermophoresis diffusion coefficient. R_{f} is the retardation factor, defined as the ratio of the agent velocity to the interstitial fluid velocity. V represents the gradient operator.

### Ultrasound Induces Interstitial Fluid Hydrostatic Pressure Increase (P₂) Via Ultrasound Momentum Transfer

Starting from mass conservation (i.e., continuity equation), momentum conservation and the Navier-Stokes equation (eventually reduced to Darcy's law equation), equation 1 in Table 1 can be derived. It indicates that the increase of the hydrostatic pressure of the interstitial fluid (P₂) is due to a non-zero value of the divergence of the ultrasound momentum density (∇·(ρ₁V₁)). Further, ∇·(ρ₁V₁) is a negative value because ultrasound momentum is lost and transferred to tissue's motions. Physically, it means that the momentum of the ultrasound wave is partially transferred to (or absorbed by) tissue and is converted into a local hydrostatic pressure rise of the tissue fluid. Mathematically, it can be further expressed as the right-hand side part of the equation 1. Clearly, P₂ depends on α, a tissue's ultrasound absorption coefficient, and a term of [∇P̅₁·∇P̅₁*] that is the dot product between the gradient of P̅₁ and its conjugate. Additionally, P̅₁ is the 1^{st}-order complex amplitude of the ultrasound pressure and ∇P̅₁ is the gradient of P̅₁, and ∇P̅₁* is the complex conjugate of ∇P̅₁. Thus, P₂ is proportional to [∇P̅₁·∇P̅₁*], rather than pressure itself.

Elevated Hydrostatic Pressure Increase (P₂) induces the exudation fluid velocity (W₂)

Once P₂ is calculated from the equation 1, based on Darcy's law, equation 2a indicates that the velocity (W₂) of the exudation fluid relative to the solid matrix is determined by tissue's hydraulic conductivity (K) and the gradient of P₂ (i.e. ∇P₂). A typical value of W₂ can be close to or higher than the typical value of the natural flow velocity in the peripheral area of a tumor (0.1-1 micron/s). This exudation fluid generates a controllable flow, which functions as a convection transport mechanism and bring agents out of the focal volume. It is much more efficient and controllable than merely altering tissue permeability and diffusivity.

### Turning Off Ultrasound Leads to a Backflow Velocity (W₂) of the Fluid Because of the Recovery of the Solid Tissue's Deformation Generated in Previous Step

When ultrasound is turned off, the driving force is removed (i.e. [∇P̅₁·∇P̅₁*]=0), and P₂ decays to zero almost immediately because of the extremely fast decay speed of the hydrostatic pressure relaxation. The compressed solid matrix tends to expand and recover to its original position at a velocity Vₛ because of its elasticity (i.e. the apparent modulus H). This expansion sucks the fluid back into the focal volume because any expanded volume must be filled by fluid, assuming no void space occurs in tissue. The initial recovery velocity Vₛ is usually high because of tissue's high apparent modulus (H) and can be calculated via the bi-phase model of the equation 2b in Table 1. \vec{u}ₛ is the displacement of the solid matrix from their original position and Vₛ is its velocity. Once Vₛ is calculated, W₂ is known based on equation 2b. The simulation results showed that a portion of the fluid can quickly flow back into the original focal volume, but the rest of the fluid will very slowly flow back (FIG. 3D and FIG. 5E-G, I-K). The quick recovery of tissue fluid will allow for the repetition of this procedure without mechanical tissue damage from the significant loss of tissue fluid in the focal volume. Additionally, the slow backflow limits the recovery of the agents and therefore allows for the continuous pumping of the agents out of the focal volume via an accumulation effect.

Ultrasound Induces Tissue Temperature Rise (ΔT) from Energy Absorption

For safety precautions, the maximum temperature increase ΔT can be limited to a pre-set value of ΔT₀, which is usually regulated by a thermal index threshold. Also, the gradient of ΔT can affect agent distribution via thermophoresis. Therefore, ΔT can be calculated using the bio-heat transfer equation, which is equation 3a in Table 1. When ultrasound is off, the decay of ΔT is described via the equation 3b by simply setting |P̅₁|=0. As an example, FIG. 4A shows the change of ΔT as a function of time at the center of the ultrasound focus with an exposure time of t₀=10 s and an ultrasound amplitude of |P̅₁|=1 MPa. Clearly, during the period between 0 and 10 s, when the ultrasound is turned on, ΔT quickly rises from 0 to 4.87° C., but the increase rate is gradually reduced because of the thermal loss caused by thermal diffusion and blood perfusion, as indicated by the first and the second terms on the right hand side of the equation 3b in Table 1, respectively.

FIG. 4B shows the 2D distribution of ΔT on the xz plane when t=t₀=10 s. The lateral (x) and axial (z) FWHMs of ΔT are 1.4 and 6.5 mm, respectively, which are wider than those of |P̅₁| in FIG. 2A (0.5 and 3.5 mm, respectively) because of the existing thermal diffusion. This expansion in space would be favorable for the purpose of controlling agent distribution if the thermophoresis was one of the dominant mechanisms. This is because a larger space with ΔT compared with the space of |P̅₁| will be more efficient at affecting agent distribution. In equation 3, cₜ and c_{b} are the specific heat capacities of tissue and blood, respectively. kₜ is tissue's thermal conductivity. ρ₀ and ρ_{b} are the density of tissue and blood, respectively. ω_{b} is the blood perfusion rate. c₀ is the sound speed in tissue.

### Ultrasound Induces Changes in Agent Concentration Via Three Mechanisms

Once W₂ and ΔT are calculated from equation 2 and 3, the dynamic change of agent concentration can be calculated based on the continuity equation (equation 4 in Table 1). C is the agent concentration. J is the total flux due to agent transport via the three mechanisms, including diffusion due to the concentration gradient (−D_{BD}∇C), thermophoresis due to ultrasound-induced temperature gradient (−CD_{TP}∇(ΔT)), and ultrasound-induced flow of interstitial fluid (CR_{f}W₂). σₛ and σᵢ are the rate of source and sink of the agents. For simplicity, it was assumed σₛ=σᵢ=0. This indicates no extra sources and sinks that can create and absorb the agents. D_{BD} and D_{TP} are the agent diffusion coefficient and the thermophoresis diffusion coefficient, respectively. R_{f} is the retardation factor, defined as the ratio of the agent velocity to the interstitial fluid velocity.

### C. Simulated Results of Agent Re-Distribution Via SIF-TUM

### Temperature-Insensitive Agents

In this study, for simplicity, the agent diameter and the tissue pore diameter have Gaussian distributions. Their standard deviations diameters are 25% of their average diameters. Whenever the following parameters are constant, their values will be set as following: the spatial peak value |P̅₁| of 0.8 MPa; the ultrasound exposure time t of 40 s (therefore, the spatial peak temperature increase ΔT of 4.15° C.); the hydraulic conductivity K of 4×10⁻¹⁴ m⁴/N/S; the apparent module H of 1 MPa; the agent average diameter of 40 nm with a 25% standard deviation (i.e., 10 nm) to represent a typical nanoparticle size; the tissue pore average diameter of 80 nm with a 25% standard deviation (i.e., 20 nm). Thus, the majority of the agents is smaller than the majority of the pores, and they should be able to pass through the pores without significant resistance. The lateral FWHM is 0.5 mm along both x- and y-directions. The axial FWHM is 3.5 mm along the z-direction.

FIG. 5A-B respectively show the change in the normalized concentration of the agent (C/C₀) on the xy and xz planes at the time right after the ultrasound is off (i.e. t=t₀=40 s). FIG. 5C-D show the results at the time of 60 s after ultrasound is turned off (i.e. t=t₀+60=100 s). FIG. 5E-F respectively plot the normalized concentration changes along x- and z-direction at different times. FIG. 5G shows the dynamic change of the normalized concentration as a function of time at the ultrasound focal center with different nanoparticle average diameters. FIG. 5K displays the effect of the nanoparticle average diameter on the normalized concentration at 40 s and 100 s. FIG. 5I-J show the effect of the tissue apparent module (H) on the relaxation of the normalized concentration change at the center of the focus after ultrasound is off. The nanoparticle average diameter is 1.6 nm and 80 nm in FIG. 5I-J, respectively. FIG. 5H displays the change rates of the normalized concentration caused by different mechanisms: diffusion, thermophoresis, and ultrasound-induced convection rate.

From the above results, it can be seen that during the period when the ultrasound is on (0≤t≤40 s), the agent nanoparticles are continuously “squeezed” out of the focal volume (FIG. 5A-G). The concentration in the surrounding areas should increase (i.e., C/C₀>1) but may not be visible within the figures because of the large surrounding volume. The maximum concentration reduction at the center of the ultrasound focus can reach approximately 4.5% (i.e., reduced from 100% to 95.5%) at 40 s. Since the ultrasound-induced convection rate is significantly higher than the diffusion and thermophoresis rates (FIG. 5K), not intending to be bound by theory, the ultrasound-induced convection is the major driving force during this period. From FIG. 5H, it can be seen that when the ultrasound is on, the ultrasound-induced convection rate is almost a constant. However, it is slightly reduced, which is due to the remaining fluid in the focal volume being slowly reduced when fluid is continuously “squeezed” out. Therefore, the concentration is approximately reduced linearly as a function of time during this period (FIG. 5G). After the ultrasound is turned off, the normalized concentration (C/C₀) quickly recovers, reaches a relatively stable value, and then very slowly decays (FIG. 5G). Based on FIG. 5E-H, it can be seen that the initial backflow speed is high (i.e., right after the ultrasound is off), which leads to a small portion of concentration recovery in the focal volume. However, when t>50 s, it becomes so slow that the agent concentration recovery is almost negligible during this time frame (except for the small agents, with diameters of 1.5 and 4 nm, and their concentration changes are not only caused by backflow but also by diffusion). Also, it is notable that a ring shape of concentration is observed in FIGS. 5C-D and 5E-F, which means that more agents have accumulated surrounding the focal area after ultrasound is turned off.

FIG. 5K indicates that the nanoparticles with a diameter between 10 and 40 nm achieve a preferred concentration reduction when the average diameter of the tissue pores is 80 nm. This means that within this diameter range, the nanoparticles not only freely pass through the tissue pores but also avoid significant natural diffusion because of their medium size. When the diameter is larger than 40 nm, especially larger than 80 nm, the resistance of tissue to the transport of the nanoparticles become significant, and then the concentration change becomes difficult. On the other hand, when the diameter is smaller than 10 nm, although these agents can easily pass through the pores, they can also diffuse back into the focal volume more efficiently than the larger ones, which reduces the efficiency of concentration change. However, even with this disadvantage of diffusion-caused backflow for small sized agents, such as 1.5 and 4 nm, their concentration changes are still higher than those of the large sized agents, such as >80 nm.

FIG. 5I-J indicates that the tissue relaxation is also slightly affected by the apparent modulus (H). The higher value of H provides the higher initial speed of backflows and therefore reaches the final value quicker. Again, the continuous concentration reduction at the late stage in FIG. 5I is mainly due to the diffusion of the small agent compared with the large agent in FIG. 5J. FIG. 5H indicates the contribution of each mechanism. Clearly, the ultrasound-induced convection rate is much larger than the diffusion rate and thermophoresis rate during both periods of the ultrasound being on and off.

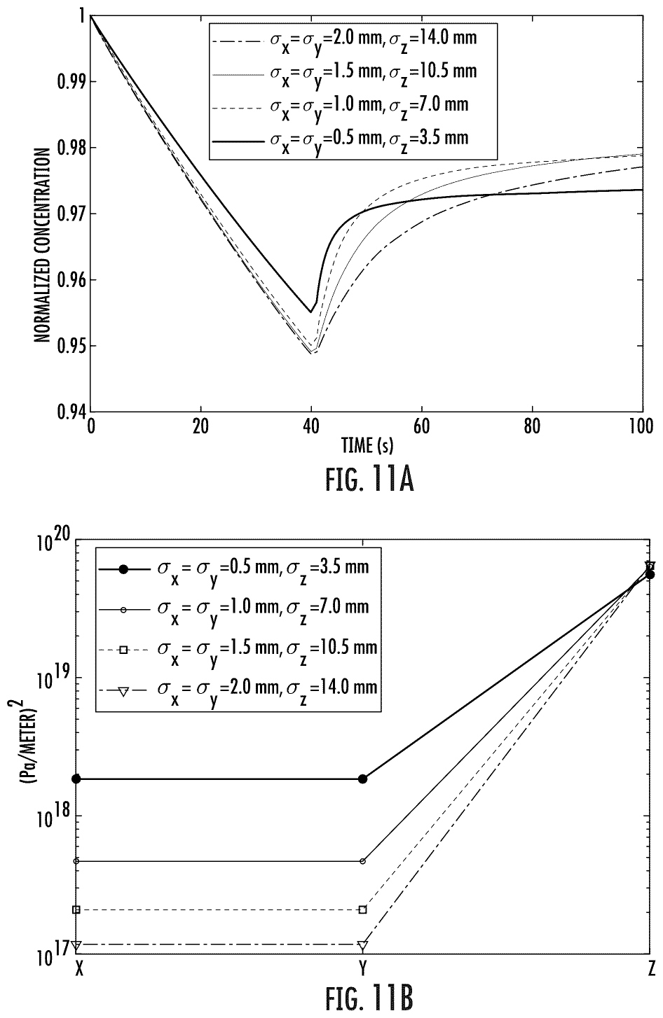

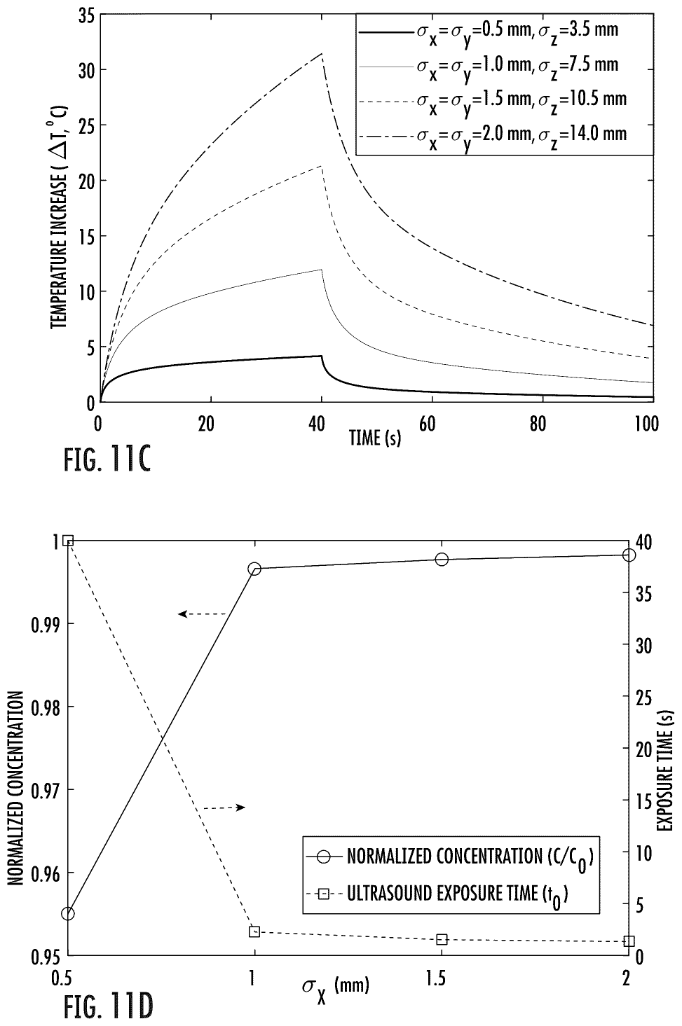

How the ultrasound focal size affects the concentration change (C/C₀) was also assessed. The σₓ(=σ_{y}) was increased from 0.5 to 2.0 mm with a step size of 0.5 mm, and the Oz was increased from 3.5 to 14 mm with a step size of 3.5 mm. The concentration change was calculated using the same methods discussed above while keeping all the other parameters were the same. FIG. 11A shows the concentration change at the focal center compared to time. It was found that the increase of FWHMs only slightly affected the concentration change. The gradient of pressure is expressed [∇P̅₁·∇P̅₁*]. To understand this, [∇P̅₁·∇P̅₁*] can be expressed as

$$[ {( \frac{\partial R}{\partial x} )}^{2} + {( \frac{\partial I}{\partial x} )}^{2} ] + [ {( \frac{\partial R}{\partial y} )}^{2} + {( \frac{\partial I}{\partial y} )}^{2} ] + [ {( \frac{\partial R}{\partial z} )}^{2} + {( \frac{\partial I}{\partial z} )}^{2} ] ,$$

where R and I are the real and imaginary parts of P̅₁, respectively. The part in each square bracket, i.e.

$$[ {( \frac{\partial R}{\partial x} )}^{2} + {( \frac{\partial 1}{\partial x} )}^{2} ] , [ {( \frac{\partial R}{\partial y} )}^{2} + {( \frac{\partial 1}{\partial y} )}^{2} ] and [ {( \frac{\partial R}{\partial z} )}^{2} + {( \frac{\partial 1}{\partial z} )}^{2} ] ,$$

corresponds to the component that contributes to the total value of [∇P̅₁·∇P̅₁*] along the x-, y- and z-directions, respectively. FIG. 11B shows the values of these three components for different FWHMs. Clearly, it is shown that the contributions from the components along the x- and y-directions to the total value of [∇P̅₁·∇P̅₁*] are much smaller than that from the component along z direction, even though the σₓ and σ_{y} are as small as 0.5 mm.

To further understand this, how long of a distance is needed for the pressure amplitude is reduced from the peak to its half (in calculation, it should be the real and imaginary parts of P̅₁ or P̅₁* to be calculated) can be evaluated and compared. In general, the distance is a ⅛ of an ultrasound wavelength along the z direction (i.e.

$$\frac{λ}{8} = 0.6 \; 16 / 8 = 0.0 \; 77 mm$$

when using a 2.5-MHz ultrasound wave with a sound speed of 1,540 meters per second in tissue). On the other hand, the needed distance is a half of the FWHM along the x- or y-direction (i.e. σₓ/2=σ_{y}/2=0.25, 0.5, 0.75, and 1 mm). Clearly, the needed distance along the z-direction is much shorter. Therefore, the component along z direction contributes more to [∇P̅₁·∇P̅₁*]. In addition, the square operation in each component will further amplify the contribution difference between the z- and x- or y-direction. Therefore, it seems that increasing the lateral focal size of the ultrasound beam does not hurt the driving force and the concentration change.

If there are no other concerns, a large ultrasound beam can be used because it can transport agents in a large volume at each location in the same ultrasound exposure time and therefore improve the efficiency and reduce the total operation time. Also, a large focal volume can reduce the backflow velocity to improve the transport efficiency (FIG. 11A). This is because the recovery rate of the tissue solid frame is smaller in a larger volume, since the recovery is a diffusion-based process (FIG. 11A). However, a large focal volume will lead to a thermal risk concern because the ultrasound-induced temperature ΔT will be significantly increased compared with that from a small focal volume when all the other parameters are the same. FIG. 11C shows that the peak ΔT can reach 4.15° C., 11.95° C., 21.31° C. and 31.42° C. at the focal center at t=t₀=40 s when Ox increases from 0.5 to 1, 1.5, and 2 mm, respectively. This is because the thermal diffusion rate is lower in a large focal volume compared to that in a small focal volume. This is understandable because that the thermal energy is more difficult to spread out from a large volume compared with from a small volume. Therefore, if the peak ΔT is limited at a fixed and safe value, such as 4.15° C., either the amplitude of the ultrasound pressure (P̅₁) or the exposure time (t₀) or both can be reduced, which will sacrifice the driving force and therefore the transport efficiency. Thus, a balance between the focal size and P̅₁ or to can be used when maintaining the ΔT fixed.

As an example, here, ΔT was limited to 4.15° C. by shortening the exposure time to while fixing |P̅₁| at 0.8 MPa, which was the same value as before). FIG. 11D shows the results. Clearly, when σₓ increases from 0.5 mm to 1 mm, 1.5 mm, and 2 mm, the exposure time t₀ has to be reduced from 40 s to 2.272 s, 1.527 s, and 1.341 s, respectively, to maintain ΔT at 4.15° C. Meanwhile, the corresponding normalized concentration is 0.955, 0.9966, 0.9977 and 0.9983, respectively.

In conclusion, if thermal risk is not a concern, such as in a thermal ablation application, a large focal size will be beneficial for improving the transport efficiency. However, if thermal risk is a concern and the peak ΔT has to be limited below a certain value, a small focal size will be preferred for balancing between efficiency and safety. The specific focal size needs can be selected based on the limit of ΔT. Lastly, another benefit of using a small focal size is that it can help to achieve a higher spatial resolution when scanning a targeted volume. This feature is notable for treating some special areas where the high spatial accuracy is required, such as in the brain where the surrounding functional areas should be avoided or in a location where a large blood vessel or nerve should be avoided.

FIG. 6 shows three examples showing how the concentration changes when applying multiple ultrasound foci or scanning on the xy plane when z=0. FIG. 6A displays the normalized concentration distribution on the xy plane when the ultrasound foci are distributed in a square from 0 to 1.5 mm with a step size of 0.5 mm along both the x and y directions. The dots indicate the focus locations. It can be seen that the concentration in the area is reduced and the agents are pushed outside the scanned area. Thus, there is the capability to control the agent distribution externally. FIG. 6B-C display similar results but with two random distributions of the ultrasound foci. It can be clearly seen that the concentration of the agents can be controlled by controlling the ultrasound focus locations, which can be done via mechanical or electronic scanning or simultaneously generate multiple foci. Other parameters are similar to those in FIG. 5, including a diameter of 40 nm, |P₁| of 0.8 MPa, to of 40 seconds, H of 1 MPa, ΔT of 4.15° C., K of 4×10⁻¹⁴ m⁴/N/S, FWHM of 0.5, 0.5 and 3.5 mm along the x-, y- and z-directions, respectively.

### Temperature-Sensitive Agents

USF imaging contrast agents are temperature-sensitive particles. Usually, their diameters are reduced around 2 or more than 2 times when the environment temperature is increased slightly above a temperature threshold, such as the lower critical solution temperature of the material (LCST). Compared with temperature-insensitive nanoparticles, this uniqueness can be favorable for enhancing the transport efficiency for large particles.

As mentioned in previous section, when the average size of the agent is close to or bigger than the average size of tissue pores, the transportability of the agent in tissue is significantly reduced, which is unfavorable for controlling the concentration. However, when using temperature-sensitive agents, this issue can be solved. This is because ultrasound will heat the tissue in the focal volume a few degrees such that ΔT is a few degrees, the environment temperature in this focal volume is slightly above the threshold (i.e. LCST), and the agent significantly shrinks. Thus, the transportability of the agent in the focal volume will be improved, and the more agents can be “squeezed” out of the focal volume. After these agents are “squeezed” out of the focal volume, their size will recover to the original size because the environment temperature outside the focus is equal or close to the background temperature that is lower than the threshold. Thus, if the original size is bigger than the pore size, the agents will not be able to flow back into the focal volume after ultrasound is turned off.

After ultrasound is turned off the temperature in the focal volume will be more quickly decayed back to the background temperature before a significant amount of interstitial fluid backflows into the focal volume. This is because the thermal diffusion rate is usually higher than the backflow velocity. Thus, the environment temperature in the focal volume after ultrasound is off will be lower than the threshold (i.e. LCST), and the agent will remain the original size and may not be able to freely backflow into the focal volume via the convection mechanism.

FIGS. 7A and 7B shows the 2D distribution of the normalized concentration on xy and xz planes, respectively, right after ultrasound is turned off (t=t₀=40 s). FIG. 7C-D display the similar results when t=100 s. FIG. 7E-F shows the 1D distribution of normalized concentration along the x and z directions at different times, respectively. The major difference compared to the temperature-insensitive agents is that the average diameter of the agent in FIG. 7A-F is 80 nm, while it is 40 nm in FIG. 5A-F. When comparing FIG. 7A-F with FIG. 5A-F, it can be found that the concentration changes are similar in two situations. Therefore, a qualitative conclusion can be drawn that using a temperature-sensitive agent, such as an USF agent, allows a larger size to achieve similar performances in concentration change compared with using a temperature-insensitive agent. This can be beneficial for improving delivery efficiency because a larger sized agent will have a bigger volume and therefore a larger payload capacity for larger molecules.

Similar to FIG. 5G, FIG. 7G plots the dynamic variation of the normalized concentration at the focal center as a function of time for agents with different diameters. During the period between 0 and 40 s, ultrasound is on and then off after 40 s. Compared FIG. 7G with FIG. 5G, it can be seen that for the same agent diameter, the concentration change is higher for temperature-sensitive agents than for temperature-insensitive agents.

To further quantitatively compare the concentrations in FIG. 7G and FIG. 5G, two time points, 40 s and 100 s, were selected, and the normalized concentration was plotted as a function of the agent diameter in FIG. 7H. Clearly, at 40 s, the curve of the concentration for the temperature-sensitive agents (the line with circles) is shifted toward the right-hand side compared with that for the temperature-insensitive agents (the line with inverted triangles). Similar results can also be found for 100 s (comparing the line with squares with the line with open triangles). The vertical dotted line indicates the tissue pore size is 80 nm. As discussed previously, a preferred agent size for temperature-insensitive agents can be between 10 and 40 nm for balancing between the transportability and diffusivity. A preferred agent size for temperature-sensitive agents is much widely broadened and can reach a range between 10 and 120 nm, in some embodiments.

It is worthy to note that when the agent shrinks in a high temperature area, it usually expels water molecules instead of payloads. This unique feature provides a great opportunity to transport large sized agents, such as 40-120 nm, in tissue using this SIF-TUM technology combined with temperature-sensitive agents. FIG. 7I further plots the difference of the normalized concentration between the temperature-sensitive and -insensitive agents as a function of agent diameter. It can be seen that when the diameter is ≤40 nm or ≥300 nm, both types of agents have similar concentration changes. This is understandable because when the agent has a size either much smaller or bigger than tissue pore size, the transportability of the agent is much less dependent on the size. However, when the agent has a size close to or slightly above the pore size, such as 80-200 nm in this example, the feature of thermally reducing size can help to improve the transportability. This can be seen from FIG. 7I in which the difference of the concentration change between the two types of agents is obvious for agents with a size between 80 and 200 nm. This result indicates that if the agent size is within the range of 80-200 nm, using a temperature-sensitive agent should have a higher transportability than a temperature-insensitive agent. However, when temperature-sensitive agents have been selected, using a size within the range of 10-120 nm will be a better choice compared with using an agent with a size>120 nm because the absolute value of the concentration change is higher.

FIG. 7J shows the effect of the apparent module on the tissue recovery rate. From this, it can be concluded that the higher H will lead to a faster initial recovery rate. In addition, FIG. 7J shows a higher concentration change than FIG. 5J. This further indicates that a temperature-sensitive agent has a higher transportability than a temperature-insensitive agent if the agent diameter is appropriate.

In summary, using temperature-sensitive agents, such as USF agents, with an appropriate size (e.g., close to or slightly above the tissue pore size) can significantly improve the transportability of the agent in tissue via SIF-TUM. More importantly, if USF agents are adopted, the same system and agent can be simultaneously used for two purposes: controlling agent distribution and USF imaging for monitoring the agent re-distribution in centimeters-deep tissues. USF has high-spatial resolution in centimeters-deep tissue. Thus, the agent redistribution can be accurately monitored in real time without adding additional costs or devices. The acquired USF images can provide feedback about the agent redistribution and provide guidance for the entire procedure. With feedback and guidance, the accuracy of controlling agent distribution in deep tissue can be significantly improved.

Experimental Parameters that can Significantly Affect Agent Concentration Change

Based on equations in Table 1, the following experimental parameters can significantly affect the agent concentration change.

Pressure gradient (∇P̅₁): The gradient of P̅₁ along the z direction is a contributor to

$$[ ∇ {\overline{P}}_{1} \cdot ∇ {\overline{P}}_{1}^{*} ] .$$

Therefore, reducing the ultrasound wavelength or increasing the frequency can significantly increase the driving force

$$( i . e . [ ∇ {\overline{P}}_{1} \cdot ∇ {\overline{P}}_{1}^{*} ] ) .$$

In addition, it is known that both later and axial focal sizes depend on the f-number of the ultrasound transducer. The f-number of a transducer is defined as a ratio of the focal length to the diameter of the transducer. Therefore, reducing the f-number can reduce the later and axial focal size, which can increase the gradient of P̅₁ along x, y, and z directions. However, this increase may be relatively limited compared with that via increasing frequency. However, increasing frequency can lead to higher absorption of ultrasound energy, and therefore higher temperature rise and smaller penetration depth.

The up-limit of the allowed temperature increase (ΔT₀): It is a user pre-set value for each ultrasound exposure and is mainly for safety concern. It can be adjustable based on FDA-suggested thermal index. Increasing ΔT₀ will allow increasing either |P̅₁| or to or both. Therefore, it can increase the agent concentration change when other conditions remain the same.

Balance between |P̅₁| and t₀ when ΔT₀ remains constant: When ΔT₀ is a fixed value, the ultrasound pressure amplitude |P̅₁| and the exposure time t₀ can be balanced to achieve an agent concentration change. Increasing the pressure strength |P̅₁| can be paired with reducing the exposure time t₀ or vice versa. Reducing |P̅₁| and increasing to is able to increase the agent concentration change. First, |P̅₁| in some cases can be weak enough to avoid a situation in which the pressure will quickly increase ΔT to reach the upper limit of ΔT₀ in a short time to, which can sacrifice the concentration change (see the situation (1) in FIG. 8A; the thin line). Second, |P̅₁| in some cases can be sufficiently strong so as to avoid the situation in which ΔT will reach a stable final value because of the existing of thermal diffusion that is much smaller than the upper limit of ΔT₀, which can waste the allowed space of ΔT₀ and provide a low amount of the concentration change (the situation (2) in FIG. 8A; the dot dashed line). Third, |P̅₁| in some cases can be a value that can increase the tissue temperature close or equal to ΔT₀ (the situation (3) in FIG. 8A; the bold line). Thus, the exposure time t₀ can be reasonably extended to improve the concentration change compared with situation (1) and (2).

By maintaining ΔT₀ of 4.8° C., FIG. 8B shows the simulated data of the normalized concentration of the agent (C/C₀) at the center of the ultrasound focus as a function of the pressure amplitude |P̅₁| at different exposure time to. Clearly, by reducing the pressure |P̅₁| from 2 to 0.8 MPa, the exposure time t₀ can be extended from 0.2 to 80 seconds (see the line with squares in FIG. 8B). Accordingly, the agent concentration change (ΔC/C₀) at the center of the ultrasound focus can increase from 0.17% to 7% (see the line with circles in FIG. 8B). Reducing the pressure well below 0.8 MPa can further allow for increasing the exposure time to, but ΔT will have difficulty reaching 4.8° C. because of the existing of thermal diffusion. FIG. 8C shows a similar example when ΔT₀ of 2.5° C. In general, a similar trend can be found. However, a lower ΔT₀ will lead to a much smaller ΔC/C₀. For example, for ΔT₀ of 2.5° C. and |P̅₁| of 0.8 MPa, the exposure time t₀ can only be extended to 4.2 seconds, which leads to the ΔC/C₀ can only achieve 0.58% that is much smaller than 7% when ΔT₀ of 4.8° C. and |P̅₁| of 0.8 MPa.

The agent parameters include but not limited the following: (1) size, (2) charge, (3) shape, (4) type, (5) temperature sensitivity, and (6) others. The molecule is assumed to have a spherical shape, and the diameter is used to measure the size. The size effect on the agent concentration change is mainly through its effect on hydraulic conductivity (K) and retardation factor (R_{f}), instead of the diffusion coefficient (D_{BD}).

### Re-Distribution Efficiency

Two outcome parameters can be defined to quantitatively compare the re-distribution efficiency: local (E_{L}) and global (E_{G}) re-distribution efficiencies. The former (E_{L}) is defined as the time that is needed for the maximum concentration of the agent reducing to 50% of the original concentration per unit volume at a specific location in a tumor. It can expressed as

$${E}_{L} = ( {t}_{L}^{50 \%} ) / {V}_{0}$$

when ΔC/C₀=50%, in which V₀ is the approximated ultrasound focal volume in SIF-TUM.

E_{G} is defined as the total time that is needed for the averaged concentration of the agent reduced to 50% of the original averaged concentration per cm³. These two parameters are final outcome parameters and have different indications. The global re-distribution efficiency E_{G} is a macroscopic and an accumulated change of the agent concentration in a tumor. It reflects a significantly large transport of the agents in a tumor (50%).

The local re-distribution efficiency E_{L} is the agent concentration change at each scanning position, which is a local and instant parameter. It is important to understand the mechanism but may not be clinical relevant as E_{G}. Compared with other technologies, SIF-TUM much more efficiently controls the agent distribution in tumors in a reasonable time period by using safe ultrasound exposures. Under selected conditions, the local (E_{L}) and global (E_{G}) re-distribution efficiencies can be within a meaningful range that can be used for human tumors in a clinical setting.

### D. Devices and Systems

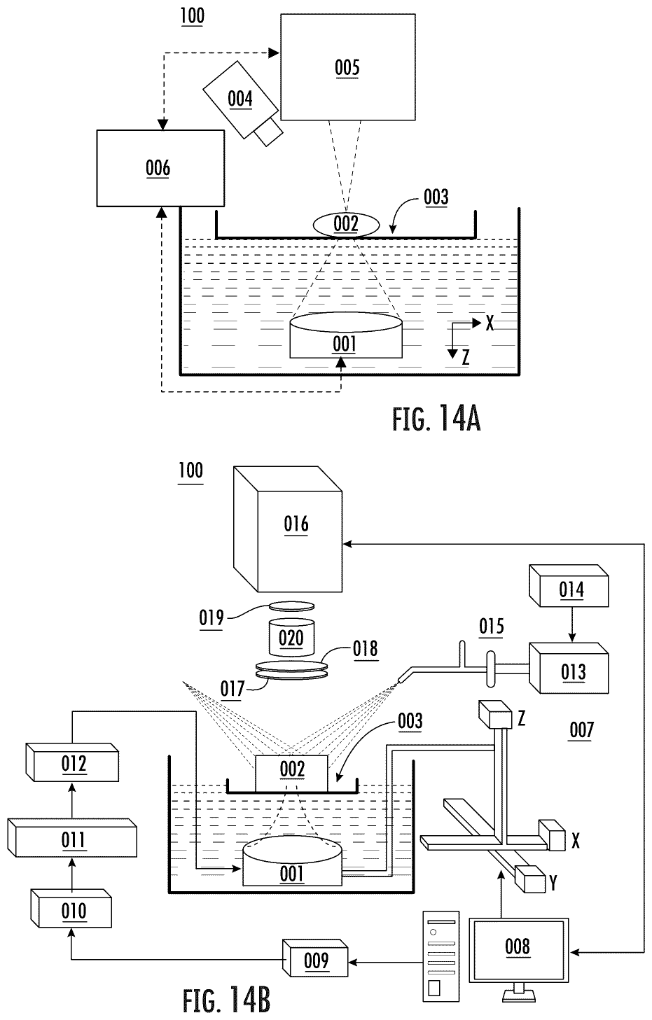

Examples of designs for SIF-TUM systems (100) are shown in FIG. 14A-B. In general, three sub-systems are included (FIG. 14A): an ultrasound system (001) submerged in water as indicated by the dashed lines, a fluorescence imaging system (005), and a control system (006). In some embodiments, the SIF-TUM can include an infrared camera (IRC) (004), as discussed below. The sample (002) sits on a stage (003).

The ultrasound system (001) is used to generate an ultrasound beam is focused in the targeted tissue for controlling the agent distribution and can be scanned 3-dimensionally. The fluorescence imaging system (005) is used to dynamically monitor the agent distribution changes caused by the ultrasound beam in the tissue. Based on different application scenarios, the fluorescence imaging system (005) can be a 3D imaging system, such as an ultrasound-switchable fluorescence (USF) imaging system for sub-millimeter-resolution and centimeters-deep tissue imaging; a fluorescence diffusion tomographic imaging system for millimeters-resolution and centimeters-deep tissue imaging; a fluorescence laminar tomographic imaging system for sub-millimeter-resolution and millimeters-deep tissue imaging; or a fluorescence microscopic imaging system, such as a confocal or multiphoton microscope, for micron-resolution and sub-millimeter-deep tissue imaging. On the other hand, for simplicity, the fluorescence imaging system (005) can also be a 2D imaging system such as a system with a regular camera or an intensified camera or an electron multiplying camera for some applications in which 3D information is not required. A control system (006) can communicate with the other systems and is used to accurately control the ultrasound exposure pressure strength (|P̅₁|) and time (t₀) via different electronic devices, control the scanning of the focus, control the fluorescence imaging and data acquisition, and so on.

In some applications, when the ultrasound-induced temperature is required be monitored, there are at least three possible ways to achieve this goal: non-invasive deep tissue temperature imaging technology, such as USF-based deep tissue thermometry or other imaging based thermometry, such as magnetic resonance imaging (MRI) or ultrasound or photoacoustic based methods; a IRC for tissue surface temperature imaging as shown in FIG. 14A; or other invasive methods, such as thermocouple- or optical fiber-based temperature sensors.

As an example, FIG. 14B shows another embodiment of a SIF-TUM-based system. In this embodiment, an ultrasound system (001) submerged in water (as indicated by the dashed lines) is present. The ultrasound is movable using a 3-D translation stage (007) and a sample (002) sits on a stage (003). A computer (008) controls the 3-D stage (007). The ultrasound system further comprises a data acquisition card (009), a function generator (010), a radio-frequency amplifier (011), and a matching network (012). The optical portion of the system includes a laser (013) with a function generator (014) and an excitation filter (015) and a gain-controlled EMCCD camera (016) with emission filters (017, 018, and 019) and a lens (020) to image the fluorescence signals.

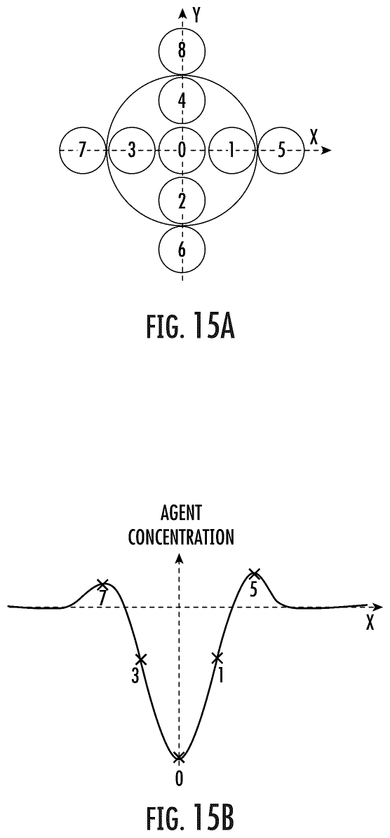

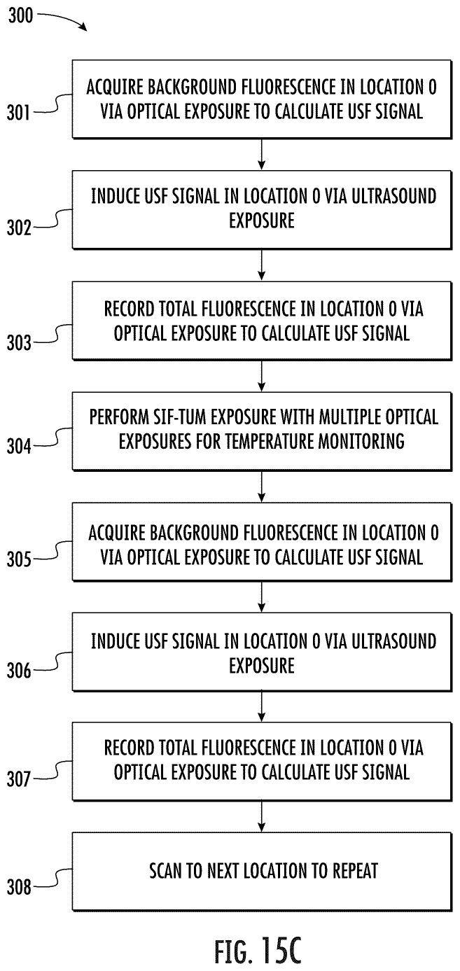

When both SIF-TUM and USF imaging are conducted in one system with the third function of temperature monitoring via USF thermometry, the system should align and accommodate the multiple functions. For example, the focal size of the ultrasound beam can be selected for accommodating each purpose. The optical exposure can be well designed relative to the ultrasound exposures and the firing sequence of the ultrasound pulses for different purposes can be designed appropriately (FIG. 15). To explain this, FIG. 15A-B show an example of how to spatially arrange the focuses for both SIF-TUM and USF imaging. Typically, the focal size of SIF-TUM is larger than the acoustic focal size of the ultrasound transducer because of the diffusion property of the hydrostatic pressure (P₂, FIG. 2B) and the thermal energy (FIG. 4). Therefore, the focal size of SIF-TUM is bigger and is represented as the larger circle in FIG. 15A. The focal size of USF imaging is small and represented as the smaller circles in FIG. 15A with a specific number to indicate the location. The USF focal size is usually close to the ultrasound acoustic focal size because the exposure time is very short and the thermal confinement is satisfied.

In practice, it is also possible to use a small focus for USF imaging and a relatively large focus for SIF-TUM via the same ultrasound transducer array. No matter which way, USF imaging can be conducted at multiple locations in and surrounding the SIF-TUM focal area, as indicated in FIG. 15A. The agent concentration change in SIF-TUM can also be estimated by only imaging the center location (position 0) via the USF method. Therefore, the focal size of the ultrasound transducer is not limiting. To further understand this, FIG. 15B shows the USF imaging locations along the x-axis on which the locations 0, 1, 3, 5 and 7 are indicated on a curve that represents the agent concentration variation induced by SIF-TUM.

FIG. 15C shows an example of the sequence of the ultrasound and optical exposures for agent re-distribution via SIF-TUM, USF imaging, and temperature monitoring, respectively. In method 300 of FIG. 15C, USF imaging is conducted at the focal volume of the SIF-TUM, location 0. Initially, in step 301, an optical exposure to acquire the background fluorescence that will be used as a baseline for that specific USF image. Right after that, in step 302, a short ultrasound exposure is conducted, which is used to induce USF signal by switching on the USF contrast agents, the same agents to be re-distributed by SIF-TUM. Immediately after that, in step 303, a second optical exposure occurs. The acquired fluorescence signal includes both the background signal and the USF signal. Thus, subtracting the first signal from the second one, only USF signal remains, which represents the signal only coming from the agents in that specific location. This signal is the USF signal that it is proportional to the agent concentration at that specific location and will be used to reflect the agent concentration changes in SIF-TUM. In step 304, a relative long ultrasound exposure is conducted to re-distribute the agents via SIF-TUM. During this exposure, it is possible to further conduct multiple optical exposures to detect the temperature changes. After the SIF-TUM exposure, the system allows the temperature fall for a very short time period, and then USF imaging will be conducted again to image the agent concentration change in steps 305, 306, and 307. Once this second USF imaging is done, the focus will be scanned to the next position in step 308 to repeat the above procedures until all the interested areas are observed. This method can be applied to all locations 1-8 in FIG. 15B.

### E. Experimental Data

### Sif-TUM Experimental Data in Live Tumors

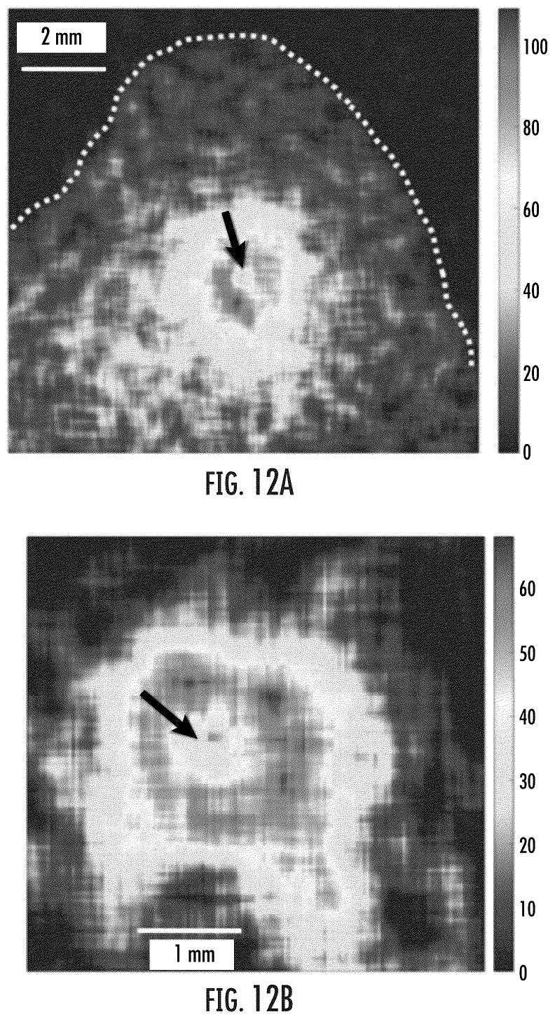

A nude mouse was subcutaneously implanted with a BXPC3 pancreatic tumor and intravenously injected with a USF contrast agent, indocyanine green (ICG)-encapsulated nanoparticles (approximately 65 nm). The ultrasound focus had a size of 0.5×0.5×3.5 mm and was positioned inside the tumor at a depth of approximately 3.5 mm. FIG. 12A-B show the ultrasound-induced change of fluorescence from the nanoparticles at two different tumor locations. The nanoparticles were temperature sensitive and should emit stronger fluorescence when temperature was increased by ultrasound. However, the fluorescence signal in the ultrasound focal area, indicated by arrows, is much less increased compared with that in the surrounding area (see the ring structures). This is because the nanoparticles are “pushed” away from the focal volume via SIF-TUM. This gives provides a unique opportunity to control the agent distribution in a tumor. Because the images were acquired right after ultrasound exposure, it avoided the possible effect of the mechanical vibration on the fluorescence signal.

SIF-TUM Data Acquired from U87 Brain Tumors

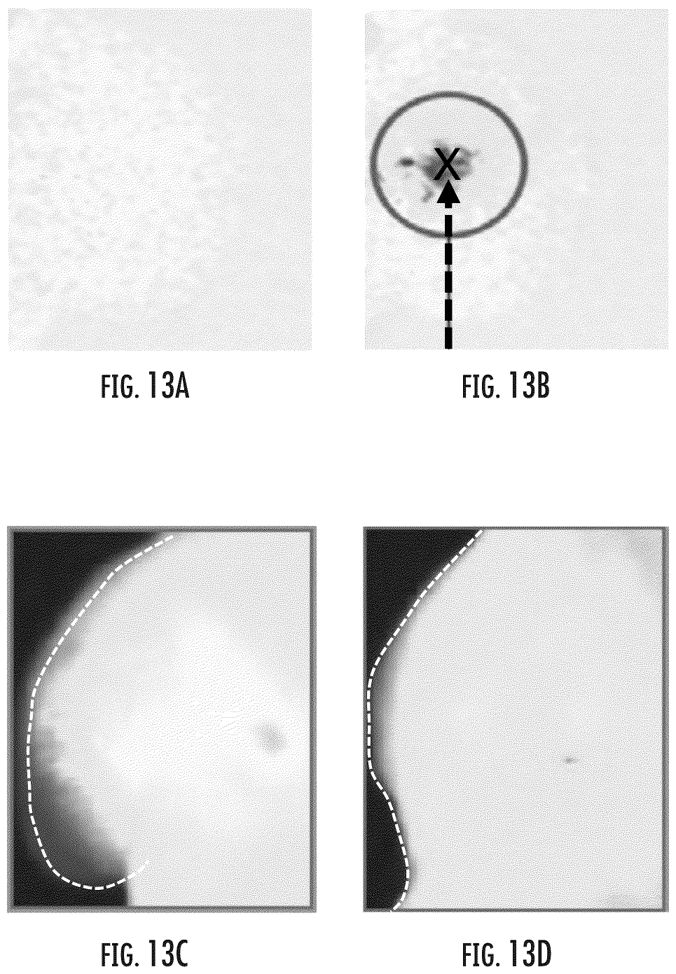

U87 brain tumors were tested similarly with USF particles of approximately 36 nm. FIG. 13A shows the background fluorescence image of the tumor without ultrasound exposure. FIG. 13B shows the fluorescence image after a 0.4-s ultrasound exposure. The circled area shows an obvious signal reduction with a much larger area than those in the ring structures in the BXPC3 tumors. To control the distribution in the entire tumor, the ultrasound focus was scanned in 3D inside the tumor. FIGS. 13C and 13D respectively show the results before and after ultrasound scanning. Clearly, the distribution changed and was relatively more uniform after ultrasound scanning.

### F. Conclusion

This Example discloses results for the monitoring of the distribution, re-distribution, feedback, and control of agents using SIF-TUM. This application will be helpful for critical applications such as diagnostic efficacy and efficiency by externally controlling agent distribution in tumors.

### G. References

1 Yao, T., Yu, S., Liu, Y. & Yuan, B. In vivo ultrasound-switchable fluorescence imaging. Sci Rep-Uk 9, 9855, doi:10.1038/s41598-019-46298-2 (2019).
2 Pei, Y. B. et al. High resolution imaging beyond the acoustic diffraction limit in deep tissue via ultrasound-switchable NIR fluorescence. Sci Rep-Uk 4, 4690, doi:Artn 4690 Doi 10.1038/Srep04690 (2014).
3 Cheng, B. B. et al. High-Resolution Ultrasound-Switchable Fluorescence Imaging in Centimeter-Deep Tissue Phantoms with High Signal-To-Noise Ratio and High Sensitivity via Novel Contrast Agents. Plos One 11, doi:ARTN e016596310.1371/journal.pone.0165963 (2016).
4 Yuan, B., Uchiyama, S., Liu, Y., K., N. T. & Alexandrakis, G. High-resolution imaging in a deep turbid medium based on an ultrasound-switchable fluorescence technique. Appl Phys Lett 101 033703 (2012).
5 Yu, S., Yao, T., Liu, Y. & Yuan, B. In vivo ultrasound-switchable fluorescence imaging using a camera-based system. Biomed Opt Express 11, 1517-1538, doi:10.1364/BOE.385996 (2020).
6 Liu, Y. et al. A Biocompatible and Near-Infrared Liposome for In Vivo Ultrasound-Switchable Fluorescence Imaging. Advanced Healthcare Materials 9, 1901457 (2020).
7 Yao, T., Liu, Y., Ren, L. & Yuan, B. Improving sensitivity and imaging depth of ultrasound-switchable fluorescence via an EMCCD-gain-controlled system and a liposome-based contrast agent. Quantitative imaging in medicine and surgery 11, 957-968 (2020).
8 Yu, S., Wang, Z., Yao, T. & Yuan, B. Near-infrared temperature-switchable fluorescence nanoparticles. Quantitative imaging in medicine and surgery 11, 1010-1022 (2020).
9 Bartelink, I. H. et al. Tumor Drug Penetration Measurements Could Be the Neglected Piece of the Personalized Cancer Treatment Puzzle. Clinical pharmacology and therapeutics 106, 148-163, doi:10.1002/cpt.1211 (2019).
10 Heldin, C.-H., Rubin, K., Pietras, K. & Östman, A. High interstitial fluid pressure—an obstacle in cancer therapy. Nature Reviews Cancer 4, 806-813, doi:10.1038/nrc1456 (2004).
11 Barua, S. & Mitragotri, S. Challenges associated with penetration of nanoparticles across cell and tissue barriers: A review of current status and future prospects. Nano Today 9, 223-243, doi:https://doi.org/10.1016/j.nantod.2014.04.008 (2014).
12 Shi, J., Kantoff, P. W., Wooster, R. & Farokhzad, O. C. Cancer nanomedicine: progress, challenges and opportunities. Nature Reviews Cancer 17, 20-37, doi:10.1038/nrc.2016.108 (2017).
13 Wilhelm, S. et al. Analysis of nanoparticle delivery to tumours. Nature Reviews Materials 1, 16014, doi:10.1038/natrevmats.2016.14 (2016).
14 Swabb, E., Wei, J. & Gullino, P. Diffusion and Convection in Normal and Neoplastic Tissues. Cancer Res 34, 2814-2822 (1974).
15 Jang, S. H., Wientjes, M. G., Lu, D. & Au, J. L. S. Drug Delivery and Transport to Solid Tumors. Pharmaceutical Research 20, 1337-1350, doi:10.1023/A: 1025785505977 (2003).
16 Jain, R. Physiological Barriers to Delivery of Monoclonal Antibodies and Other Macromolecules in Tumors. Cancer Res 50, 814s-819s (1990).
17 Baxter, L. T. & Jain, R. K. Transport of fluid and macromolecules in tumors. I. Role of interstitial pressure and convection. Microvasc Res 37, 77-104, doi:https://doi.org/10.1016/0026-2862 (89) 90074-5 (1989).
18 Jain, R. K. Transport of Molecules, Particles, and Cells in Solid Tumors. Annu Rev Biomed Eng 1, 241-263, doi:10.1146/annurev.bioeng.1.1.241 (1999).
19 Giordano, S. et al. 3D Mass Spectrometry Imaging Reveals a Very Heterogeneous Drug Distribution in Tumors. Sci Rep-Uk 6, 37027, doi:10.1038/srep37027 (2016).
20 Boucher, Y., Brekken, C., Netti, P. A., Baxter, L. T. & Jain, R. K. Intratumoral infusion of fluid: estimation of hydraulic conductivity and implications for the delivery of therapeutic agents. British Journal of Cancer 78, 1442-1448, doi:10.1038/bjc. 1998.705 (1998).
21 Swartz, M. A. & Fleury, M. E. Interstitial Flow and Its Effects in Soft Tissues. Annu Rev Biomed Eng 9, 229-256, doi:10.1146/annurev.bioeng.9.060906.151850 (2007).
22 Baxter, L. T. & Jain, R. K. Transport of fluid and macromolecules in tumors. II. Role of heterogeneous perfusion and lymphatics. Microvasc Res 40, 246-263, doi:https://doi.org/10.1016/0026-2862 (90) 90023-K (1990).
23 Di Paolo, A. & Bocci, G. Drug distribution in tumors: mechanisms, role in drug resistance, and methods for modification. Current oncology reports 9, 109-114, doi:10.1007/s11912-007-0006-3 (2007).
24 Hong, W. X. et al. Intratumoral Immunotherapy for Early-stage Solid Tumors. Clin Cancer Res 26, 3091-3099, doi:10.1158/1078-0432.ccr-19-3642 (2020).
25 Perrin, J. et al. Cell Tracking in Cancer Immunotherapy. Frontiers in Medicine 7, doi:10.3389/fmed.2020.00034 (2020).
26 Bell, J. & McFadden, G. Viruses for Tumor Therapy. Cell Host and Microbe 15, 260-265 (2014).
27 Dolmans, D. E. J. G. J., Fukumura, D. & Jain, R. K. Photodynamic therapy for cancer. Nature Reviews Cancer 3, 380-387, doi:10.1038/nrc1071 (2003).
28 Li, Y. et al. Phototherapy using immunologically modified carbon nanotubes to potentiate checkpoint blockade for metastatic breast cancer. Nanomedicine: Nanotechnology, Biology and Medicine 18, 44-53, doi:https://doi.org/10.1016/j.nano.2019.02.009 (2019).
29 Chen, W., Adams, R., Higgins, A. K., Bartels, K. & Nordquist, R. Photothermal effects on murine mammary tumors using indocyanine green and an 808-nm diode laser: an in vivo efficacy study. Cancer letters 98 2, 169-173 (1996).
30 Ren, L., Liu, Y., Yao, T. & Yuan, B. Tumor ultrasound-switchable fluorescence imaging. In preparation (2021).
31 Curley, C. T. et al. Augmentation of brain tumor interstitial flow via focused ultrasound promotes brain-penetrating nanoparticle dispersion and transfection. Science Advances 6, eaay 1344, doi:10.1126/sciadv.aay 1344 (2020).
32 Arvanitis, C. D. et al. Mechanisms of enhanced drug delivery in brain metastases with focused ultrasound-induced blood-tumor barrier disruption. Proceedings of the National Academy of Sciences 115, E8717-E8726, doi:10.1073/pnas. 1807105115 (2018).
33 O'Neill, B. E. et al. Pulsed High Intensity Focused Ultrasound Mediated Nanoparticle Delivery: Mechanisms and Efficacy in Murine Muscle. Ultrasound in Medicine & Biology 35, 416-424 (2009).
34 Khaibullina, A. et al. Pulsed High-Intensity Focused Ultrasound Enhances Uptake of Radiolabeled Monoclonal Antibody to Human Epidermoid Tumor in Nude Mice. Journal of Nuclear Medicine 49, 295-302, doi:10.2967/jnumed. 107.046888 (2008).
35 Wang, S. et al. Pulsed high intensity focused ultrasound increases penetration and therapeutic efficacy of monoclonal antibodies in murine xenograft tumors. Journal of Controlled Release 162, 218-224, doi:https://doi.org/10.1016/j.jconrel.2012.06.025 (2012).
36 O'Neill, B. E. & Li, K. C. in Encyclopedia of Nanotechnology (ed Bharat Bhushan) 2181-2186 (Springer Netherlands, 2012).
37 Nieminen, H. J. et al. in 2012 IEEE International Ultrasonics Symposium. 1869-1872.
38 Larina, I. V. et al. Enhancement of Drug Delivery in Tumors by Using Interaction of Nanoparticles with Ultrasound Radiation. Technology in Cancer Research & Treatment 4, 217-226, doi:10.1177/153303460500400211 (2005).
39 Pitt, W. G., Husseini, G. A. & Staples, B. J. Ultrasonic drug delivery—a general review. Expert Opinion on Drug Delivery 1, 37-56, doi:10.1517/17425247.1.1.37 (2004).
40 Couture, O., Foley, J., Kassell, N. F., Larrat, B. & Aubry, J.-F. Review of ultrasound mediated drug delivery for cancer treatment: updates from pre-clinical studies. Translational Cancer Research 3, 494-511 (2014).
41 Tharkar, P., Varanasi, R., Wong, W. S. F., Jin, C. T. & Chrzanowski, W. Nano-Enhanced Drug Delivery and Therapeutic Ultrasound for Cancer Treatment and Beyond. Frontiers in Bioengineering and Biotechnology 7, doi:10.3389/fbioe.2019.00324 (2019).
42 El Ghamrawy, A. et al. Acoustic Streaming in a Soft Tissue Microenvironment. Ultrasound in Medicine & Biology 45, 208-217, doi:https://doi.org/10.1016/j.ultrasmedbio.2018.08.026 (2019).
43 Løvmo, M. K. et al. Effect of Acoustic Radiation Force on Displacement of Nanoparticles in Collagen Gels. IEEE Transactions on Ultrasonics, Ferroelectrics, and Frequency Control 68, 416-431, doi:10.1109/TUFFC.2020.3006762 (2021).
44 Yeh, H.-L. & Juárez, J. J. Ultrasound-enhanced diffusion and streaming of colloids in porous media. Experimental Thermal and Fluid Science 121, 110282, doi:https://doi.org/10.1016/j.expthermflusci.2020.110282 (2021).
45 Nieminen, H., Ylitalo, T., Suuronen, J.-P. & Serimaa, R. Delivering Agents Locally into Articular Cartilage by Intense MHz Ultrasound. Ultrasound in Medicine & Biology 41, 2259-2265 (2015).
46 Ranjan, A. et al. Image-guided drug delivery with magnetic resonance guided high intensity focused ultrasound and temperature sensitive liposomes in a rabbit Vx2 tumor model. Journal of Controlled Release 158, 487-494, doi:https://doi.org/10.1016/j.jconrel.2011.12.011 (2012).
47 Yuh, E. L. et al. Delivery of Systemic Chemotherapeutic Agent to Tumors by Using Focused Ultrasound: Study in a Murine Model. Radiology 234, 431-437, doi:10.1148/radiol.2342030889 (2005).
48 Ziadloo, A., Xie, J. & Frenkel, V. Pulsed focused ultrasound exposures enhance locally administered gene therapy in a murine solid tumor model. The Journal of the Acoustical Society of America 133, 1827-1834, doi:10.1121/1.4789390 (2013).
49 Lee, E. S. et al. Pulsed high-intensity focused ultrasound enhances apoptosis of pancreatic cancer xenograft with gemcitabine. Ultrasound in Medicine & Biology 39, 1991-2000 (2013).
50 Pitt, W. G. Ultrasound in Drug and Gene Delivery. Adv Drug Deliver Rev 60, 1095-1096 (2008).
51 Frenkel, V. Ultrasound mediated delivery of drugs and genes to solid tumors. Adv Drug Deliver Rev 60, 1193-1208, doi:https://doi.org/10.1016/j.addr.2008.03.007 (2008).
52 Tebebi, P. A. et al. Improving the therapeutic efficacy of mesenchymal stromal cells to restore perfusion in critical limb ischemia through pulsed focused ultrasound. Sci Rep-Uk 7, 41550, doi:10.1038/srep41550 (2017).
53 Hancock, H. et al. Investigations into Pulsed High-Intensity Focused Ultrasound-Enhanced Delivery: Preliminary Evidence for a Novel Mechanism. Ultrasound in Medicine & Biology 35, 1722-1736 (2009).
54 de Smet, M., Heijman, E., Langereis, S., Hijnen, N. M. & Grüll, H. Magnetic resonance imaging of high intensity focused ultrasound mediated drug delivery from temperature-sensitive liposomes: An in vivo proof-of-concept study. Journal of Controlled Release 150, 102-110, doi:https://doi.org/10.1016/j.jconrel.2010.10.036 (2011).
55 Deckers, R. & Moonen, C. T. W. Ultrasound triggered, image guided, local drug delivery. Journal of Controlled Release 148, 25-33, doi:https://doi.org/10.1016/j.jconrel.2010.07.117 (2010).
56 Park, S. M. et al. Novel temperature-triggered liposome with high stability: Formulation, in vitro evaluation, and in vivo study combined with high-intensity focused ultrasound (HIFU). Journal of Controlled Release 170, 373-379, doi:https://doi.org/10.1016/j.jconrel.2013.06.003 (2013).
57 Dromi, S. et al. Pulsed-High Intensity Focused Ultrasound and Low Temperature-Sensitive Liposomes for Enhanced Targeted Drug Delivery and Antitumor Effect. Clin Cancer Res 13, 2722, doi:10.1158/1078-0432.CCR-06-2443 (2007).
58 Grüll, H. & Langereis, S. Hyperthermia-triggered drug delivery from temperature-sensitive liposomes using MRI-guided high intensity focused ultrasound. Journal of Controlled Release 161, 317-327, doi:https://doi.org/10.1016/j.jconrel.2012.04.041 (2012).
59 Sarvazyan, A., Rudenko, O. & Nyborg, W. Biomedical Applications of Radiation Force of Ultrasound: Historical Roots and Physical Basis. Ultrasound in Medicine & Biology 36, 1379-1394 (2010).
60 Yuan, B., Yao, T., Ren, L. & Liu, Y. Theoretical studies of externally controlling the distribution of molecules and nanoparticles in live tumors via a tightly focused ultrasound. In preparation (2021).
61 Yao, T., Ren, L. & Yuan, B. Experimental studies of externally controlling the distribution of molecules and nanoparticles in live tumors via a tightly focused ultrasound. In preparation (2021).
62 Sapozhnikov, O. A. An exact solution to the Helmholtz equation for a quasi-Gaussian beam in the form of a superposition of two sources and sinks with complex coordinates. Acoustical Physics 58, 41-47, doi:10.1134/S1063771012010216 (2012).
63 Aglyamov, S., Karpiouk, A. B., Linskii, Y., Zabolotskaya, E. & Emelianov, S. Motion of a solid sphere in a viscoelastic medium in response to applied acoustic radiation force: Theoretical analysis and experimental verification. The Journal of the Acoustical Society of America 122, 1927-1936 (2007).
64 Sarvazyan, A., Rudenko, O., Swanson, S., Fowlkes, J. B. & Emelianov, S. Y. Shear wave elasticity imaging: a new ultrasonic technology of medical diagnostics. Ultrasound in Medicine & Biology 24, 1419-1435 (1998).
65 Wu, J. Acoustic Streaming and Its Applications. Fluids 3, doi:10.3390/fluids3040108 (2018).
66 Doherty, J. R., Trahey, G. E., Nightingale, K. R. & Palmeri, M. L. Acoustic radiation force elasticity imaging in diagnostic ultrasound. IEEE Transactions on Ultrasonics, Ferroelectrics, and Frequency Control 60, 685-701, doi:10.1109/TUFFC.2013.2617 (2013).
67 Bruus, H. Acoustofluidics 2: Perturbation theory and ultrasound resonance modes. Lab on a Chip 12, 20-28, doi:10.1039/C1LC20770A (2012).
68 Bruus, H. Acoustofluidics 1: Governing equations in microfluidics. Lab on a Chip 11, 3742-3751, doi:10.1039/C1LC20658C (2011).
69 Bruus, H. Acoustofluidics 7: The acoustic radiation force on small particles. Lab on a Chip 12, 1014-1021, doi:10.1039/C2LC21068A (2012).
70 Zhang, Y., Czerwonka, L., Tao, C. & Jiang, J. J. A biphasic theory for the viscoelastic behaviors of vocal fold lamina propria in stress relaxation. The Journal of the Acoustical Society of America 123, 1627-1636, doi:10.1121/1.2831739 (2008).
71 Kwan, M. K., Michael Lai, W. & Van Mow, C. Fundamentals of fluid transport through cartilage in compression. Annals of Biomedical Engineering 12, 537-558, doi:10.1007/BF02371448 (1984).
72 Ehlers, W. & Markert, B. A Linear Viscoelastic Biphasic Model for Soft Tissues Based on the Theory of Porous Media. Journal of Biomechanical Engineering 123, 418-424, doi:10.1115/1.1388292 (2001).
73 Mansour, J. M. Biomechanics of Cartilage. http://web.mit.edu/cortiz/www/3.052/3.052CourseReader/27_BiomechanicsofCartilage.pdf 66-79.
74 Wongsuwarn, S. et al. Giant thermophoresis of poly(N-isopropylacrylamide) microgel particles. Soft Matter 8, 5857-5863, doi:10.1039/C2SM25061F (2012).
75 Vyas, R. & Rustgi, M. L. Green's function solution to the tissue bioheat equation. Medical Physics 19, 1319-1324 (1992).
76 Anand, A. & Kaczkowski, P. J. NONINVASIVE MEASUREMENT OF LOCAL THERMAL DIFFUSIVITY USING BACKSCATTERED ULTRASOUND AND FOCUSED ULTRASOUND HEATING. 34, 1449-1464 (2008).
77 Liu, R. et al. Temperature-sensitive polymeric nanogels encapsulating with β-cyclodextrin and ICG complex for high-resolution deep-tissue ultrasound-switchable fluorescence imaging. Nano Research 13, 1100-1110, doi:10.1007/s12274-020-2752-6 (2020).
78 Raghavan, R. Theory for acoustic streaming in soft porous matter and its applications to ultrasound-enhanced convective delivery. Journal of therapeutic ultrasound 6, 6, doi:10.1186/s40349-018-0114-6 (2018).
79 Yuan, B. et al. A system for high-resolution depth-resolved optical imaging of fluorescence and absorption contrast. Rev Sci Instrum 80, 043706, doi:10.1063/1.3117204 (2009).
80 Burgess, S. A., Bouchard, M. B., Yuan, B. & Hillman, E. M. Simultaneous multiwavelength laminar optical tomography. Opt Lett 33, 2710-2712, doi:174796 [pii] (2008).
81 Yuan, B. Radiative transport in the delta-P1 approximation for laminar optical tomography Journal of Innovative Optical Health Sciences 2, 149-163 (2009).

### EXAMPLE 3

### Assessment of the Effect of Ultrasound Frequency on Concentration Change and Transport Efficiency

For the application of SIF-TUM, increasing frequency can lead to the higher absorption of ultrasound energy and a higher temperature increase. For safety reasons in clinical applications, and if desired, the maximum temperature increase ΔT can be limited to a preset value of ΔT₀, which is ultimately regulated by a thermal index, which should be below the AIUM-required safety threshold for medicinal purposes (TI<6; American Institute of Ultrasound in Medicine). This Example presents an assessment of the effects of ultrasound on transport efficiency. The selection of frequency, which may affect the selection of other variables such as exposure time and the maximum temperature increase induced by ultrasound, also affects the efficiency of the transport of USF agents. This work will be meaningful in the establishment of parameters for the clinical application of SIF-TUM.

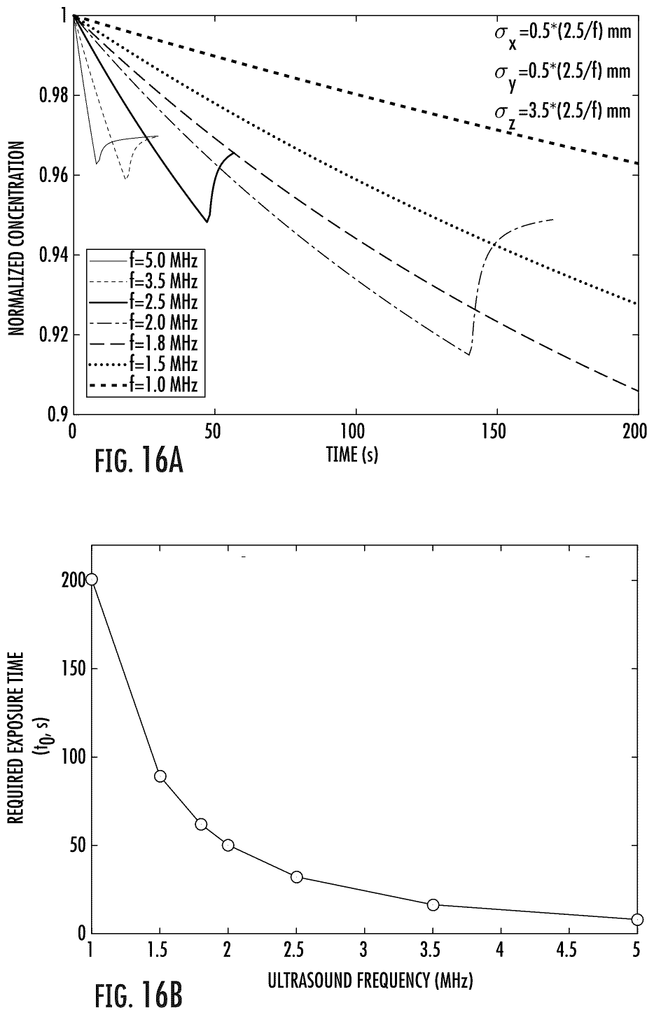

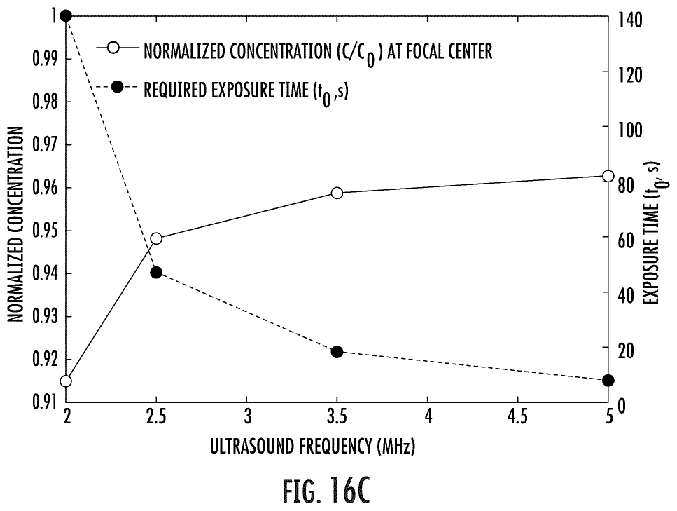

FIG. 16A displays a plot of the normalized concentration at the ultrasound focal center as a function of time(s) for different frequency from 1 to 5 MHZ. Thus, it is a study of how the ultrasound frequency affects the concentration change at the ultrasound focal center as a function of time. In this study, the maximum temperature increase induced by ultrasound (ΔT₀) is limited to 4.3° C. when the frequency is 2 MHZ, 2.5 MHZ, 3.5 MHz and 5 MHz. Thus, the ultrasound will be turned off once ΔT₀ reaches 4.3° C. When the frequency is respectively reduced to 1.0, 1.5 and 1.8 MHz, the temperature dynamic change reaches a plateau, which is 2.79° C. for 1 MHZ, 3.57° C. for 1.5 MHz, and 4.08° C. for 1.8 MHz, and always lower than the limit of 4.3° C. Thus, theoretically, the exposure time (to) of the ultrasound at the three frequencies of 1.0 MHz, 1.5 MHz, and 1.8 MHz can be as long as a user wants. However, practically, one has to consider another factor, the efficiency of concentration changes, will be sacrificed significantly when frequency is reduced, which can be seen from the figure. In addition, because the FWHMs of the focal size are inversely propositional to the frequency, the FWHMs at 2.5 MHz were used as the baseline (i.e. σₓ=σ_{y}=0.5 mm and σ_{z}=3.5 mm) and were scaled for each frequency by dividing them by the frequency. For example, the FWHMs at 5 MHz is

$${σ}_{x} = {σ}_{y} = \frac{0.5}{5} = 0.1 mm and {σ}_{z} = \frac{3.5}{5} = 0.07 mm .$$

FIG. 16A indicates that the speed or efficiency of the concentration change is highly frequency-dependent, which is indicated by the slope of each curve. In general, increasing the frequency can dramatically increase the concentration change speed or efficiency. For example, the curve at 5 MHz is much sharper than that at 1 MHZ, and therefore, it requires a much shorter time to achieve the same concentration change compared with the curve at 1 MHz. As an example, FIG. 16B shows the required exposure time (t₀) to make the normalized concentration reduced from 1 to 0.9628 at different frequencies while maintaining ΔT₀≤4.3° C. Clearly, the required exposure time exponentially decays from 200.8 s to 8 s when increasing the frequency from 1 to 5 MHz. Therefore, using higher frequency can increase the transport efficiency. This is because the driving force of SIF-TUM is proportional to the term of [∇P̅₁·∇P̅₁*], which is related to the gradient of the ultrasound pressure wave. When increasing the frequency, the pressure gradient along the axial direction (i.e. z-direction) will be increased, and therefore, the term of [∇P̅₁·∇P̅₁*] will be increased even more because of the dot product of ∇P̅₁ and ∇P̅₁*.

In practice, it is possible that one may care more about the total concentration change in a reasonable time period than the transport efficiency. The total concentration change not only depends on the transport efficiency, but also the allowed exposure time (to). A higher frequency has a higher tissue absorption coefficient and will lead to a higher temperature increase. Therefore, the total exposure time may be limited. In contrast, by increasing the exposure time, a lower frequency can achieve a higher concentration change, as shown in FIG. 16A. As an example, when ΔT₀ remained at 4.3° C., FIG. 16C shows that for 2 MHz, the concentration is reduced from 1 to 0.9149 with an exposure time of 140 s, while it can only reduce to 0.9628 for 5 MHz with an exposure time of 8 s (limited by the ΔT₀=4.3° C.). Therefore, a lower frequency has less limitation in ΔT₀ and exposure time, which is beneficial for achieving a higher concentration change. In addition, a higher frequency, such as 5 MHz in this study, will also sacrifice the penetration depth of ultrasound in tissue because of its higher tissue absorption compared with a lower frequency, such as 1-2.5 MHz. This is an important fact when considering a deep tissue intervention.

More factors can be considered when trying to select a frequency. Significantly lowering the frequency may make the transport efficiency so low that a very long exposure time is needed to achieve a targeted concentration change. Thus, using a lower frequency may be less attractive when compared with using a higher frequency with multiple exposures, as long as waiting long enough time to allow the temperature falls down between two adjacent exposures. More critically, when the frequency is too low to generate a high enough driving force and thus flow speed in tissue compared with the tissue natural flow speed, the goal of controlling the distribution of agents at these locations will fail. When a large volume is needed to scan, one may not only consider the time being spent at each location but also the total number of positions to be scanned. A larger focal size can reduce the total number of the scanned positions. Accordingly, the fact that a lower frequency has a larger focal size can help to reduce the total scanning time.

Finally, it can be seen that it is possible to adopt a relatively low frequency with a relatively long exposure time and a relatively large focal size or a relatively high frequency with multiple short exposures and a relatively small focal size. It will be understood based on the present disclosure that a selection can be made by the skilled person based on a specific application. For example, a high frequency (>3 MHz) with multiple short exposures may be preferred when the targeted intervention region is small, not too deep, weakly absorbing ultrasound, or has a high natural flow speed of interstitial fluid, such as in the tumor periphery area, or a high spatial resolution is needed to avoid interacting with surrounding vital tissues, such as nerves, blood vessels, important functional tissue, etc. In contrast, a low frequency (<1.8 MHz) with a long exposure may be selected when the volume is very large, too deep, or highly absorbing to ultrasound or has a low natural flow speed in tissue (estimated or otherwise known). A balanced frequency range (1.8-2.5 MHz) may be used when the situation is between the two extremes or not enough tissue information is available. This is because both the temporal and spatial properties are balanced in this range.

This Example discloses results that show how the choice of ultrasound frequency affects concentration change and transport efficiency. Considerations for other variables in a clinical setting for the application of SIF-TUM, such as exposure time and the maximum temperature increase induced by ultrasound, are closely related. This study will make a useful contribution to clinical applications of SIF-TUM.

### EXAMPLE 4

Measuring Recovery Time Constant as an Indicator of Cellularity in Model Systems using SIF-TUM

### A. Introduction

Early assessment of the response of a tumor to a treatment, such as chemotherapy, radiotherapy or targeted therapy, is very important and beneficial for tailoring patient's treatment plans to achieve better outcomes. While responsive patients may continue the treatment with an appropriate dose, switching to other available treatments for non- or less-responsive patients is needed to avoid the unnecessary side effects caused by an ineffective treatment. Accordingly, monitoring and assessing cancer early response to a treatment is highly desirable.

To accurately monitor and assess tumor early response, it is known that detecting cancer cellularity is important. This is because the majority of current chemotherapeutic agents adopt a therapeutic mechanism of directly killing cancer cells via cytotoxicity, and only a few are targeting tumor blood vessels via the mechanism of anti-angiogenesis. Unfortunately, most current imaging technologies, such as ultrasound and X-ray imaging, target the changes in the physical or physiological properties of tumors, such as tumor size, shape, structure, stiffness, blood vessels, and so on. However, these changes are usually much later than the change of cancer cellularity (i.e., density), which limits the sensitivity and specificity of these technologies.

Nuclear imaging, such as positron emission tomography (PET), detects cell metabolic rate via radioactive tracers. Magnetic resonance (MRI)-based diffusion-weighted imaging (DWI) detects the diffusivity of water molecules that is related to cancer cellularity. Both PET and DWI are widely believed more specific than ultrasound and x-ray technologies. Unfortunately, neither PET nor DWI is ideal for monitoring purposes because of the adoption of radioactive materials (PET), high cost and bulky equipment. Also, DWI is limited by its low signal-to-noise ratio and spatial resolution.

As a monitoring system, in the in vivo USF imaging of mouse tumors, squeezing interstitial fluid via transfer of ultrasound momentum (SIF-TUM) was applied. Compared with USF imaging, increasing the ultrasound exposure time, the exposure strength, or both, it was found that a small portion of USF contrast agents can be pushed away from the ultrasound focal volume. SIF-TUM uses a focused ultrasound to gently “squeeze” tissue in a small ultrasound focal volume from all the directions (˜0.7 mm³) via momentum transfer from the ultrasound wave to the interstitial fluid. It can be imagined as a scenario of “3-dimensionally compressing and relaxing a small internal portion of a water-filled sponge.” When ultrasound is turned on, a portion of interstitial fluid is “squeezed” out of focal volume and the nanoagents dissolved in the fluid is also “squeezed” out via convection. This phenomenon can be measured from the reduction of USF signal. When ultrasound is turned off, a portion of these “pumped-out” nanoagents can flow back into the original ultrasound focal volume. This phenomenon can be measured from the recovery of USF signal after ultrasound exposure.

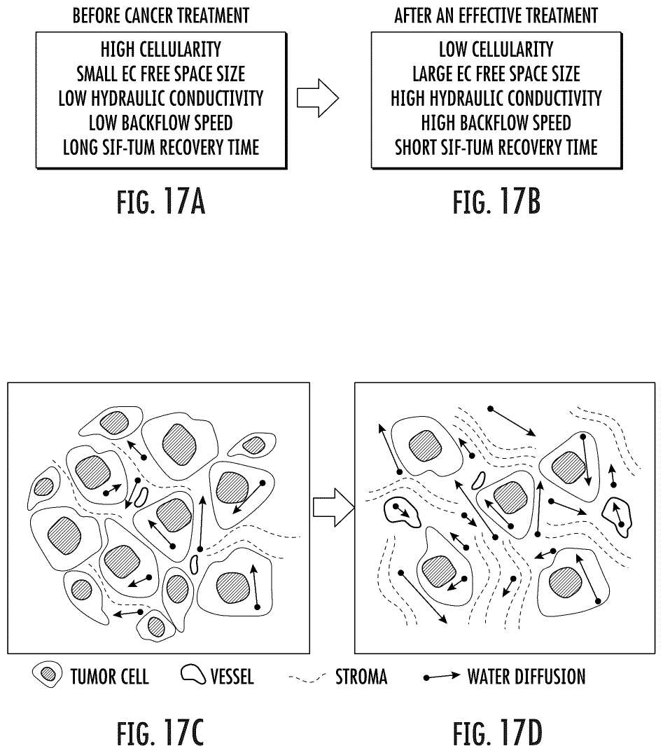

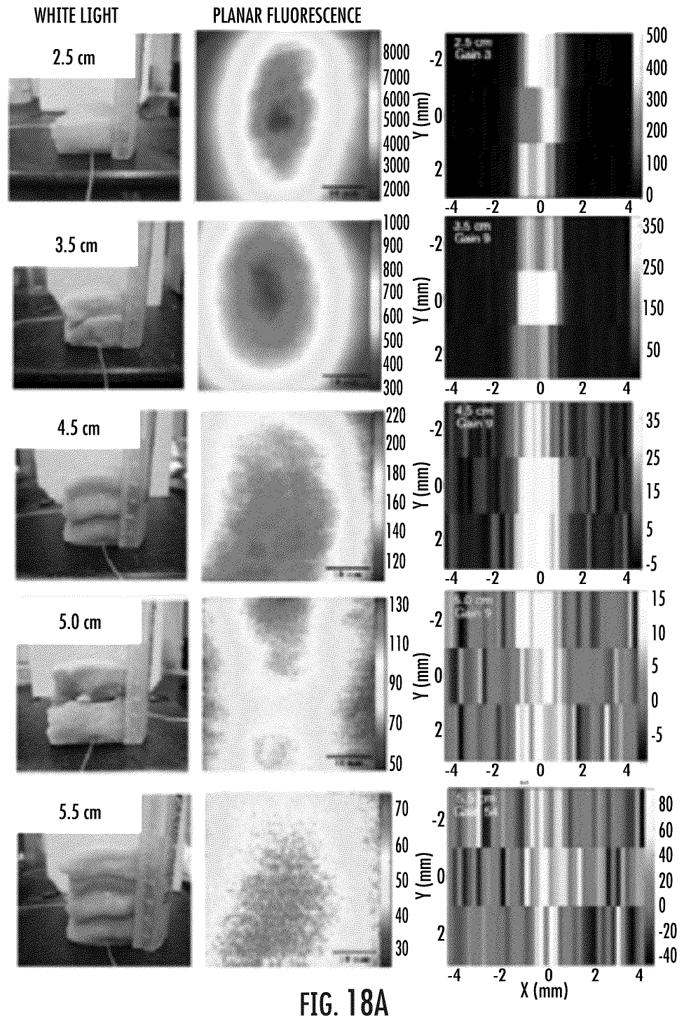

A recovery time constant (τ) of the USF signal can be defined to quantify the average time the fluid takes to flow back to the original position via an exponential decay function of y=−A*exp (−t/τ)+y₀. In this equation, y is the dynamic USF fluorescence signal during the recovery period, τ is the recovery time constant, which is independent of the absolute value of the signal strength, and A and y₀ are two constants related to signal strength but their values are independent of τ. The longer τ is, the slower the backflow speed is. FIG. 17A-B summarize the principle, and FIG. 17C-D illustrate how cellularity changes upon cancer treatment. Briefly, τ mainly depends upon tissue's properties, including cellularity (i.e., cell density or the free space size of the extracellular matrix (ECM)) via tissue's hydraulic conductivity, the interstitial fluid viscosity, and tissue's apparent modulus (related to elasticity of ECM). Most current therapies are either cytotoxic or target cancer cells, rather than ECM. Thus, for a cytotoxic chemotherapy, not intending to be bound by theory, the cellularity changes to the treatment should be much more significant than the change of the fluid viscosity and elasticity of ECM. Thus, again, not intending to be bound by theory, the recovery time constant t of USF signal is mainly affected by the change of tumor cellularity via a change in the hydraulic conductivity.

In this Example, this disclosure presents data supporting and methods to establish the recovery time constant τ of USF signal after SIF-TUM as an indicator of a change in tumor cellularity via a change in hydraulic conductivity in a collagen model system and mouse models of tumor growth. This work is helpful in the foundation of the use of the recovery time constant τ as a clinical indicator of early tumor response to cancer treatment.

### B. Data

### USF Images

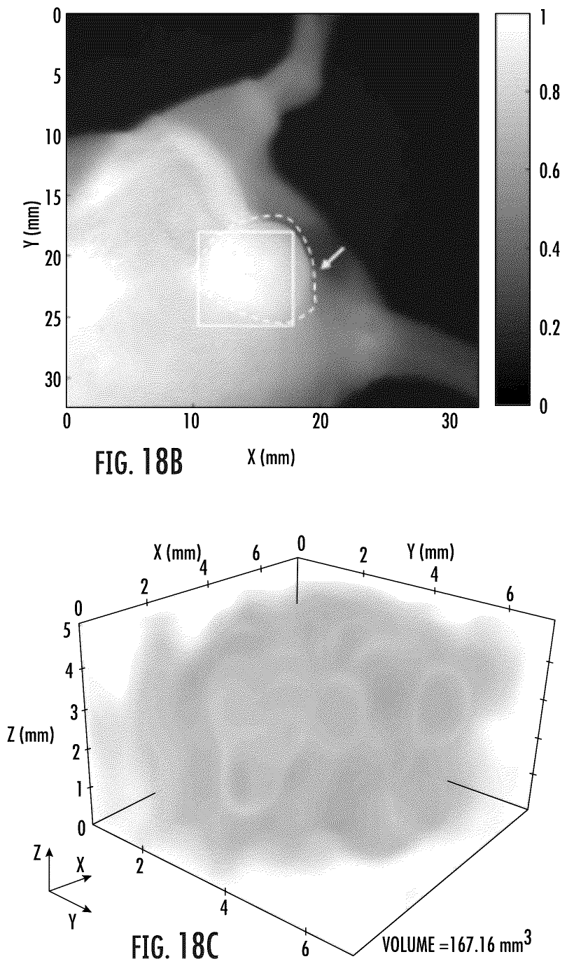

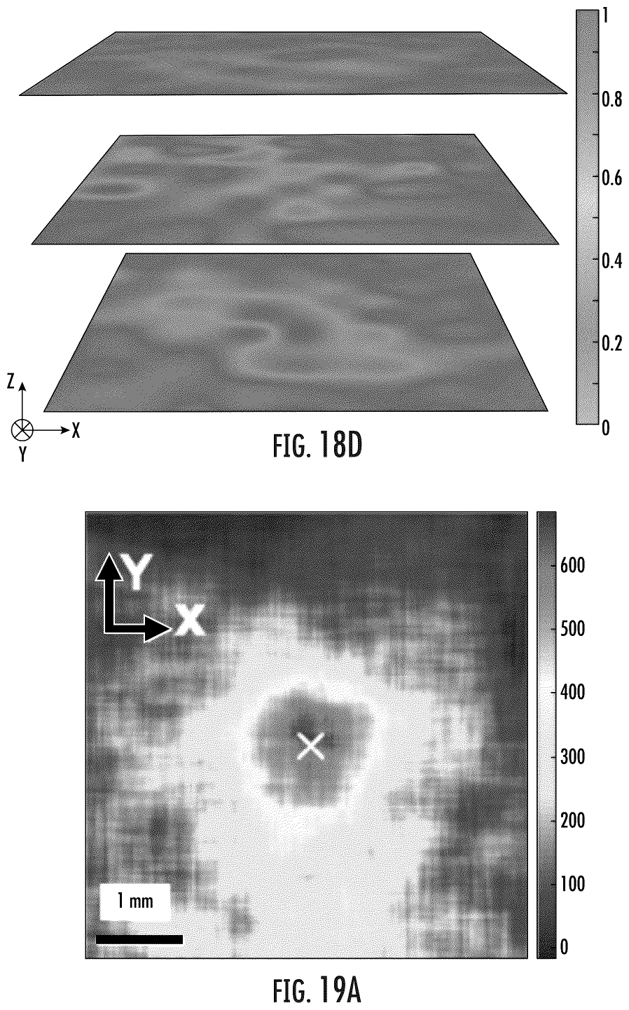

FIG. 18A shows white light, planar fluorescence, and USF images of a micro-tube containing a USF agent (inner diameter=0.76 mm) embedded in chicken breast tissue. FIG. 18B shows planar fluorescence image of the BXPC3 tumor taken at 6 hours after the intravenous injection of the same USF contrast agents. FIG. 18C is 3D USF images that show the heterogeneous distribution of the USF contrast agents in the tumor of FIG. 18B. FIG. 18D is three 2D image slices at three different depth (z) to show the distribution of the USF agents from FIG. 18C. A near infrared USF contrast agent, indocyanine green (ICG)-encapsulated temperature-sensitive nanoparticles, was adopted in both cases. Clearly, USF significantly improves spatial resolution compared with planar fluorescence imaging. Also, the distribution of the USF agents is heterogeneous in the tumor, which cannot be seen from the conventional planar image. Therefore, USF not only significantly improves the spatial resolution but also provides the 3D distribution of the USF agents. It can monitor the dynamic change of the nanoagent distribution changes induced by SIF-TUM.

### SIF-TUM-Induced In Vivo Concentration and USF Signal Reduction

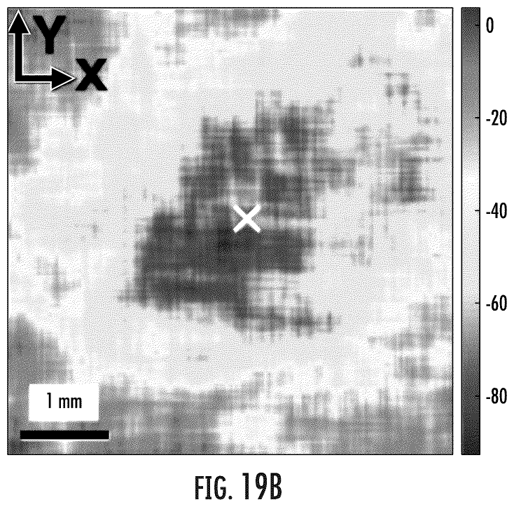

FIG. 1 displays the experimental setup. In 200, the set up includes a focused ultrasound transducer (201), the tumor (202), two excitation light fibers (203, 204), and a EMCCD camera (205). ICG-based USF contrast agents with a diameter ˜32 nm were intravenously injected into a nude mouse with a pancreatic tumor (BXPC3). Before an ultrasound exposure, a background fluorescence image was acquired. Then, an ultrasound exposure was applied using a focused ultrasound transducer (201). Immediately after the ultrasound exposure, another fluorescence image was acquired. By subtracting the 1^{st} image from the 2^{nd} image, the subtracted image reflects the fluorescence change caused by the ultrasound exposure. FIG. 19A-B respectively show a positive and a negative fluorescence signal. In USF imaging, ultrasound exposure is so short (≤0.4 s) that nanoagents do not have enough time to move. Thus, the ultrasound-induced tissue temperature increase in the focal volume results in a fluorescence increase from the nanoagents (FIG. 19A). When ultrasound exposure is longer and/or stronger (or the nanoagents have a lower concentration), SIF-TUM becomes dominant so that a portion of the nanoagents is squeezed out of the focal volume. This leads to a reduction in concentration and USF signal (i.e., negative signal area in FIG. 19B).

Fluorescence Signal Recovery after SIF-TUM Induction

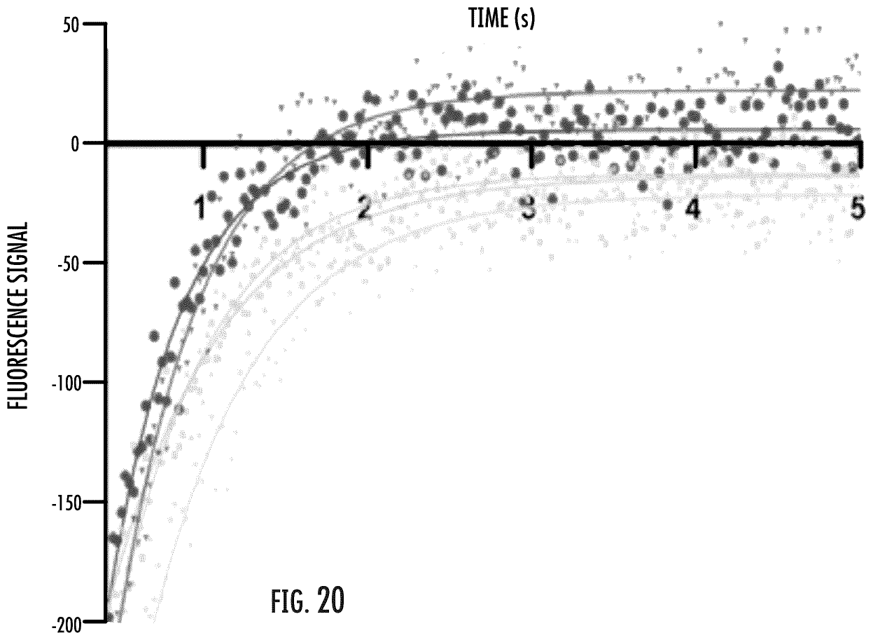

FIG. 20 shows a set of dynamic recoveries of the fluorescence signal after SIF-TUM induction. The recovery of the signal indicates that the agents are brought back to the ultrasound focal volume via the backflow of the interstitial fluid. Five sets of repeated data are shown. Although the strength of each signal is different, the recovery time constant (τ) is very close (0.58±0.09 s).

### C. Methods

### Investigating the Relationship Between t and Collagen Concentration in Gel Phantoms Via SIF-TUM Collagen Gels

Collagen gels provide a model to test the system by characterizing the relationship between t and the collagen concentration. Protocols of making collagen gels to mimic tissue with controlled concentration have been widely reported. The collagen type I solution is commercially available, and the pH and ionic strength is adjusted by NaOH and 10× phosphate-buffered saline (PBS). The polymerization of collagen solution results in the formation of fibers and filaments. Scanning electron microscopy (SEM) is used to quantify the average pore size and density.

### Fluorophore and Liposome Selection

ICG is an FDA-approved near infrared (NIR) fluorophore. ICG has been widely used in clinical settings for different diseases and has been used to image human breast cancer with a tissue penetration depth at a range of ˜5-10 cm. Liposomes have been widely used for enhancing drug delivery for human. ICG-encapsulated liposomes are biocompatible and commonly used as USF contrast agents. This type of USF agent has been well characterized. The size can be accurately controlled, and a size in the range of 50-80 nm for this study is used because they can quickly penetrate blood vessel wall and enter into tumor interstitial space.

### Imaging System Setup

FIG. 14B shows a diagram of the imaging system. Briefly, a tightly focused ultrasound transducer (001) with a focal size of 0.5×0.5×3.5 mm³ at 2.5 MHz is used for both SIF-TUM and USF exposures at different times. The ultrasound transducer is scanned using a 3D translation stage (007). The optical system includes an excitation laser (013) at 808 nm and a gain-controlled EMCCD camera with filters >830 nm (016) and is used to image the fluorescence signals. The entire system is synchronized and controlled by an in-house-made MATLAB GUI program on a computer (008). The ultrasound exposure strength and time are below the FDA-required safety thresholds of mechanical index (MI<1.9) and thermal index (TI<6). The potential temperature increase of a sample caused by ultrasound is monitored via a calibrated infrared thermal camera to remain TI<6. Ultrasound pressure is measured in samples by inserting a calibrated needle hydrophone and remains MI<1.9.

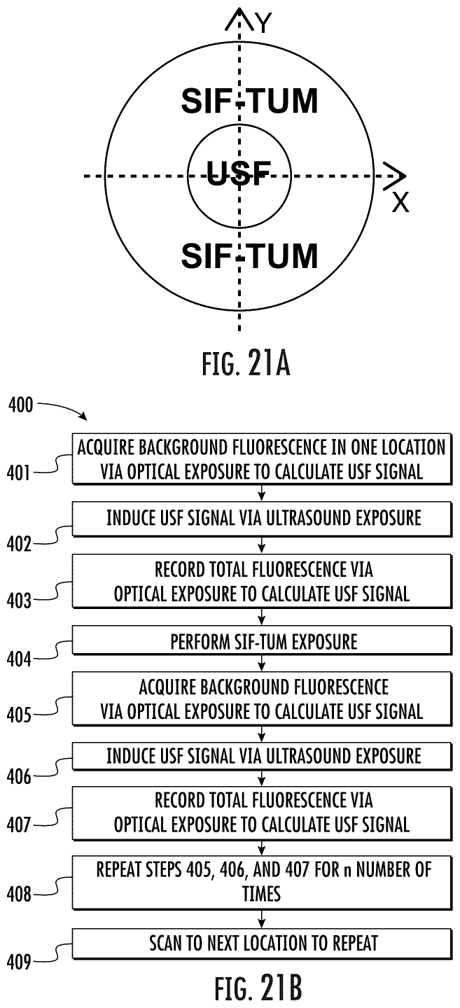

FIG. 21A shows the spatial relationship between the focal areas of the SIF-TUM and USF. The same ultrasound transducer is used for both SIF-TUM induction and USF measurements. The spot size of the SIF-TUM is bigger than the ultrasound focal size because of the diffusion property of the SIF-TUM (see equations 1-2 in Table 1). In contrast, the focal size of USF is close to the ultrasound focal size because its exposure time is so short that the thermal diffusion is limited, which is a requirement in USF.

FIG. 21B displays the sequence of the SIF-TUM and USF exposures. At each location, in method 300, initially, a USF measurement of the initial nanoagent concentration before the SIF-TUM exposure is taken. The initial USF measurement comprises an optical exposure (step 401) that records background fluorescence; a USF ultrasound exposure, which is step 401, is much shorter than the SIF-TUM exposure, and induces USF signal; and another optical exposure, which is step 303 and records the total fluorescence (i.e, background fluorescence plus USF signal). The optical image acquired from the 1^{st} optical exposure is subtracted from the optical image acquired from the 2^{nd} optical image. The spatial average of a selected area on this subtracted image is calculated and used as the strength of the USF signal to indicate the concentration of the nanoagent at this specific location. After this, at step 404, SIF-TUM exposure, which will squeeze nanoagents out of the focal volume and induce the reduction of the nanoagent concentration in the focal volume, is done. Then, in step 405, background fluorescence is acquired again. In step 406, USF signal is induced, and in step 407, the total fluorescence is recorded. In step 408, these steps can be repeated an n number of times to measure the dynamic recovery of the nanoagent concentration at different time after the SIF-TUM exposure. If the SIF-TUM effect is obvious, the USF signal reduction should be generated, and its recovery can be measured. In step 409, the scan can move to the next location to repeat the process.

After fitting the measured recovery data to the exponential decay equation of y=−A*exp (−t/τ)+y₀, the recovery time constant (τ) is extracted. Then, the ultrasound transducer is scanned to another location to repeat the same procedures. At each location, a single value for the time constant τ is established. Multiple τ for different locations can be measured by scanning the transducer. The average and standard deviation of these τ can be used to represent the tissue cellularity. By measuring the τ of a tumor before and after a treatment, the statistical change in τ reflects cancer cellularity response to the treatment. The spatial resolution is determined by the adopted ultrasound focal size and the step size of scanning. The temporal resolution depends on how long it takes at each point and the total points to be acquired.

For each gel sample, ICG-based USF nanoagent solution is locally injected into a gel. The sample is imaged via the system as shown in FIG. 6. Ultrasound strength, ultrasound exposure time, gain, and exposure time of the EMCCD are optimized to obtain the highest signal-to-noise ratio (SNR) under the safety thresholds mentioned above by varying these parameters. The data are processed to extract τ. Scanning the ultrasound focus is done at different locations to repeat and eventually achieve multiple τ values. Multiple gel samples with different collagen concentrations (i.e. different pore size and density) are measured to study the relationship of τ vs collagen concentration. The samples are imaged by SEM for quantifying the average pore size and density.

All τ data at different collagen concentrations are statistically analyzed and compared to investigate their relationship. The analysis of variance (ANOVA) test is conducted, and a p-value of less than 0.05 is considered as a significant difference. Standard deviation is used for replicated data.

### Investigating the Relationship Between τ and Tumor Cellularity in Live Mice During Tumor Growth

The commercially-available MDA-MB-231 breast cancer cell line and athymic nude female mice, which have been widely used for growing tumors, are used in this study. Two groups are included in this study: tumor and control (i.e. healthy) groups. To be able to compare the results among different groups with a confidence index (CI) 80% (a standard number), a power analysis is done to calculate the sample size. The power (CI) is set to 0.80, and the alpha is set to 0.05 (a standard number). The necessary sample size for each group is 10 for standard deviations of 0.15 (one tumor per animal).

For the tumor group, the tumor cell suspensions are injected into one of the hind legs of a mouse to grow a subcutaneous tumor. The tumor size is measured by a caliper or an ultrasound imager if the tumor grows significantly in deep tissue. Experimental parameters of ultrasound and optical are selected based on trials in 4 extra animals or tumors at different time points when the tumor diameter reaches ˜4, ˜7, ˜10, and ˜13 mm. After completion, the tissue samples are harvested and evaluated for potential damages via conventional H&E staining histopathological methods.

For the tumor group, after an intravenous injection of the liposome solution of ICG-encapsulated liposomes with a diameter between 50-80 nm (˜100 μL) via a systemic injection through the tail vein, each tumor of each mouse is imaged via the proposed method at different time points based on the circulation dynamics of the adopted ICG-encapsulated biocompatible liposomes when the diameter reaches ˜4, ˜7, ˜10, and ˜13 mm. Animals are anesthetized by isoflurane inhalation (˜3%) with oxygen and are positioned on a holder that has a hole to allow the ultrasound wave passing through and coupled into the tumor. The skin of the animal is wetted with water for ultrasound coupling. The water bath is maintained at 37° C. Ultrasound is focused into the tumor for conducting USF and SIF-TUM experiments. The optical system measures the dynamic change of the USF signal for fitting a value of τ. For each tumor, multiple t is measured at different locations in the tumor. A spatial average of t in a tumor is calculated for each tumor size.

A biopsy via a fine needle guided by ultrasound imaging is conducted after the experiment and the tissue sample is used to calculate cellularity via the conventional histopathological method. For a large tumor, the number of biopsy samples is slightly increased to avoid tumor spatial heterogeneity.

The control group aims to provide a scenario that the cellularity remains relatively unchanged during the experiment period. For the control group, the liposomes are locally injected into the muscle of a hind leg of the mouse. Similar procedures are repeated except no tumors are implanted. When a mouse with a tumor is imaged, a control mouse is paired, and similar procedures are conducted. Similar methods to those used for collagen gel studies are used to statistically analyze all data of t and cellularity in different groups. The results are compared to investigate their relationship.

Investigating how τ is Correlated to the Change of Tumor's Cellularity Induced by a Cytotoxic Chemotherapy and Whether τ is an Earlier and More Specific Indicator than Others

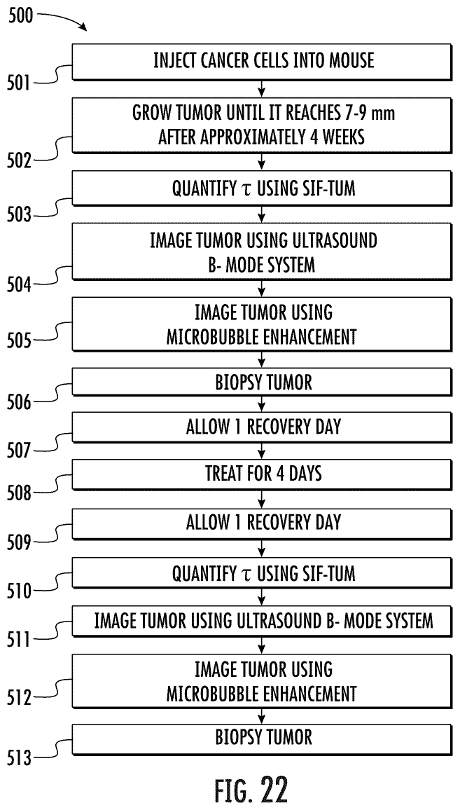

The main procedures, time sequence, and duration are listed in FIG. 22. First, at step 501, a mouse is injected with the MDA-MB-231 breast cancer cell suspensions to grow a tumor. At step 502, the tumor size grows until it reaches ˜7-9 mm (˜4 weeks). Then, before the 1^{st} treatment, at step 503, the tumor is measured via the SIF-TUM method for quantifying τ. After that, at step 504, the tumor is imaged via a commercial ultrasound (US) B-mode system. These US images are used as the background images for next step. The three dimensional sizes (x, y, z) of the tumor are quantified from this step. At step 505, a bolus of microbubbles (MB), a type of ultrasound-enhancement contrast agent, is injected via a tail vein, and a second ultrasound image is acquired after the injection. The previous background ultrasound images are subtracted from this 2^{nd} image, and the resultant images represent tumor blood perfusion. At step 506, a biopsy is conducted to evaluate cancer cellularity through conventional H&E staining. Steps 504-506 are finished in one day.

Concerning therapy selection, doxorubicin is a commonly used cytotoxic chemotherapeutic drug for many different types of cancer and is used in this study. Selecting a chemotherapy that targets cancer cells, rather than targets blood vessels, indicates that it directly kills cancer cells and changes cell density, therefore changing τ. When a cytotoxic chemotherapy is adopted, it may be unfair to compare the blood vessel related parameters, such as blood perfusion and vessel density, with t and cellularity. This is because the adopted drug is targeting cancer cells, rather than the blood vessels. However, not intending to be bound by theory, it is possible that blood vessel related parameters may also change during the cytotoxic chemotherapy. In addition, currently many researchers are investigating these blood related parameters as potential indictors even when a cytotoxic chemotherapy is used.

After the mouse recovers to a normal condition (˜1 day, step 507), the tumor is treated with an appropriate dose of doxorubicin via an injection in the experimental group. No treatment is given for animals in the control group as the treatment. Treatment lasts 4 days (step 508), with one dose being injected each day. One day after the treatment (step 509), the tumor is imaged again by following the same procedures (step 510-513) on one day. Ideally, three cycles of treatment are as described above for each mouse (or tumor).

Similar methods described for collagen gels are used to analyze values of the four indicators at different time points for both control and experiment groups, including t (via SIF-TUM), tumor size (via ultrasound), blood perfusion (via microbubble-enhanced ultrasound), and cellularity (via pathological method). Statistical comparisons among indicators and different groups are conducted.

### D. Conclusion

This Example presents data supporting and methods to establish the recovery time constant τ of USF signal after SIF-TUM as an indicator of a change in tumor cellularity via a change in hydraulic conductivity in a collagen model system and mouse models of tumor growth. This work supports the use of the recovery time constant τ as a clinical indicator of early tumor response to cancer treatment.

### E. References

1 Graham, L. J. et al. Current Approaches and Challenges in Monitoring Treatment Responses in Breast Cancer. Journal of Cancer 5, 58-68, doi:10.7150/jca.7047 (2014).
2 Gao, W., Guo, N. & Dong, T. Diffusion-weighted imaging in monitoring the pathological response to neoadjuvant chemotherapy in patients with breast cancer: a meta-analysis. World Journal of Surgical Oncology 16, 145, doi:10.1186/s12957-018-1438-y (2018).
3 Liu, S. et al. Diffusion-weighted imaging in assessing pathological response of tumor in breast cancer subtype to neoadjuvant chemotherapy. J. Magn. Reson. Imaging, 779-787 (2015).
4 Yuan, B. System and methods for improved transport and distribution of agents in biological tissue. US Provisional Patent Application Application number: U.S. 63/340,171 (May 10, 2022).
5 Avril, N., Sassen, S. & Roylance, R. Response to Therapy in Breast Cancer. Journal of Nuclear Medicine 50, 55S-63S, doi:10.2967/jnumed.108.057240 (2009).
6 Rajan, R. et al. Change in tumor cellularity of breast carcinoma after neoadjuvant chemotherapy as a variable in the pathologic assessment of response. Cancer 100, 1365-1373, doi:https://doi.org/10.1002/cncr.20134 (2004).
7 Yoshikawa, M. I. et al. Relation between cancer cellularity and apparent diffusion coefficient values using diffusion-weighted magnetic resonance imaging in breast cancer. Radiation Medicine 26, 222-226, doi:10.1007/s11604-007-0218-3 (2008).
8 Moll, U. M. & Chumas, J. Morphologic Effects of Neoadjuvant Chemotherapy in Locally Advanced Breast Cancer. Pathology-Research and Practice 193, 187-196, doi:https://doi.org/10.1016/S0344-0338 (97) 80076-3 (1997).
9 Sethi, D. et al. Histopathologic changes following neoadjuvant chemotherapy in various malignancies. International Journal of Applied and Basic Medical Research 2, 111-116, doi:10.4103/2229-516x.106353 (2012).
10 https://www.hopkinsvasculitis.org/vasculitis-treatments/cyclophosphamide-cytoxan/.
11 https://www.cancerresearchuk.org/about-cancer/cancer-in-general/treatment/cancer-drugs/drugs/carboplatin.
12 https://www.cancer.gov/publications/dictionaries/cancer-terms/def/anthracycline.
13 Wang, B. et al. Evaluation of the response of breast cancer patients to neoadjuvant chemotherapy by combined contrast-enhanced ultrasonography and ultrasound elastography. Exp Ther Med 17, 3655-3663, doi:10.3892/etm.2019.7353 (2019).
14 Sannachi, L. et al. Breast Cancer Treatment Response Monitoring Using Quantitative Ultrasound and Texture Analysis: Comparative Analysis of Analytical Models. Translational Oncology 12, 1271-1281, doi:https://doi.org/10.1016/j.tranon.2019.06.004 (2019).
15 Zhu, Q. et al. Breast Cancer: Assessing Response to Neoadjuvant Chemotherapy by Using US-guided Near-Infrared Tomography. Radiology 266, 433-442, doi:10.1148/radiol.12112415 (2013).
16 Pereira, N. P. et al. Diffusion-Weighted Magnetic Resonance Imaging of Patients with Breast Cancer Following Neoadjuvant Chemotherapy Provides Early Prediction of Pathological Response—A Prospective Study. Sci Rep-Uk 9, 16372, doi:10.1038/s41598-019-52785-3 (2019).
17 Heid, I. et al. Co-clinical Assessment of Tumor Cellularity in Pancreatic Cancer. Clin Cancer Res 23, 1461-1470, doi:10.1158/1078-0432.ccr-15-2432 (2017).
18 Guhaniyogi, S., Chu, M.-L., Chang, H.-C., Song, A. W. & Chen, N.-k. Motion immune diffusion imaging using augmented MUSE for high-resolution multi-shot EPI. Magnetic resonance in medicine 75, 639-652, doi:https://doi.org/10.1002/mrm.25624 (2016).
19 Nnewihe, A. N. et al. Custom-fitted 16-channel bilateral breast coil for bidirectional parallel imaging. Magnetic resonance in medicine 66, 281-289, doi:https://doi.org/10.1002/mrm.22771 (2011).
20 Chen, N.-k., Guidon, A., Chang, H.-C. & Song, A. W. A robust multi-shot scan strategy for high-resolution diffusion weighted MRI enabled by multiplexed sensitivity-encoding (MUSE). Neuroimage 72, 41-47, doi:https://doi.org/10.1016/j.neuroimage.2013.01.038 (2013).
21 Yao, T., Yu, S., Liu, Y. & Yuan, B. In vivo ultrasound-switchable fluorescence imaging. Sci Rep-Uk 9, 9855, doi:10.1038/s41598-019-46298-2 (2019).
22 Pei, Y. B. et al. High resolution imaging beyond the acoustic diffraction limit in deep tissue via ultrasound-switchable NIR fluorescence. Sci Rep-Uk 4, 4690, doi:Artn 4690 Doi 10.1038/Srep04690 (2014).
23 Cheng, B. B. et al. High-Resolution Ultrasound-Switchable Fluorescence Imaging in Centimeter-Deep Tissue Phantoms with High Signal-To-Noise Ratio and High Sensitivity via Novel Contrast Agents. Plos One 11, doi:ARTN e016596310.1371/journal.pone. 0165963 (2016).
24 Yuan, B., Uchiyama, S., Liu, Y., K., N. T. & Alexandrakis, G. High-resolution imaging in a deep turbid medium based on an ultrasound-switchable fluorescence technique. Appl Phys Lett 101 033703 (2012).
25 Yu, S., Yao, T., Liu, Y. & Yuan, B. In vivo ultrasound-switchable fluorescence imaging using a camera-based system. Biomed Opt Express 11, 1517-1538, doi:10.1364/BOE.385996 (2020).
26 Liu, Y. et al. A Biocompatible and Near-Infrared Liposome for In Vivo Ultrasound-Switchable Fluorescence Imaging. Advanced Healthcare Materials 9, 1901457 (2020).
27 Yao, T., Liu, Y., Ren, L. & Yuan, B. Improving sensitivity and imaging depth of ultrasound-switchable fluorescence via an EMCCD-gain-controlled system and a liposome-based contrast agent. Quantitative imaging in medicine and surgery 11, 957-968 (2021).
28 Yu, S., Wang, Z., Yao, T. & Yuan, B. Near-infrared temperature-switchable fluorescence nanoparticles. Quantitative imaging in medicine and surgery 11, 1010-1022 (2020).
29 Yuan, B. Interstitial fluid streaming in deep tissue induced by ultrasound momentum transfer for accelerating nanoagent transport and controlling its distribution. Phys. Med. Biol. Submitted (2022).
30 Saremi, B., Yao, T. & Yuan, B. Thermo- and pH-sensitive nanoparticles of poly(N-isopropylacrylamide-decenoic acid-1-vinylimidazole) for ultrasound switchable fluorescence imaging. Experimental Biology and Medicine 0, 15353702221087648, doi:10.1177/15353702221087648 (2022).
31 Ren, L., Liu, Y., Yao, T. & Yuan, B. In vivo tumor ultrasound-switchable fluorescence imaging via intravenous injections of size-controlled thermosensitive nanoparticles. Nano Research (Submitted) (2022).
32 Henke, E., Nandigama, R. & Ergün, S. Extracellular Matrix in the Tumor Microenvironment and Its Impact on Cancer Therapy. Frontiers in Molecular Biosciences 6, doi:10.3389/fmolb.2019.00160 (2020).
33 Liu, R. et al. Temperature-sensitive polymeric nanogels encapsulating with β-cyclodextrin and ICG complex for high-resolution deep-tissue ultrasound-switchable fluorescence imaging. Nano Research 13, 1100-1110, doi:10.1007/s12274-020-2752-6 (2020).
34 Sarvazyan, A., Rudenko, O., Swanson, S., Fowlkes, J. B. & Emelianov, S. Y. Shear wave elasticity imaging: a new ultrasonic technology of medical diagnostics. Ultrasound in Medicine & Biology 24, 1419-1435 (1998).
35 Zhang, Y., Czerwonka, L., Tao, C. & Jiang, J. J. A biphasic theory for the viscoelastic behaviors of vocal fold lamina propria in stress relaxation. The Journal of the Acoustical Society of America 123, 1627-1636, doi:10.1121/1.2831739 (2008).
36 Kwan, M. K., Michael Lai, W. & Van Mow, C. Fundamentals of fluid transport through cartilage in compression. Annals of Biomedical Engineering 12, 537-558, doi:10.1007/BF02371448 (1984).
37 Ehlers, W. & Markert, B. A Linear Viscoelastic Biphasic Model for Soft Tissues Based on the Theory of Porous Media. Journal of Biomechanical Engineering 123, 418-424, doi:10.1115/1.1388292 (2001).
38 Mansour, J. M. Biomechanics of Cartilage. http://web.mit.edu/cortiz/www/3.052/3.052CourseReader/27_BiomechanicsofCartilage.pdf 66-79.
39 Vyas, R. & Rustgi, M. L. Green's function solution to the tissue bioheat equation. Medical Physics 19, 1319-1324 (1992).
40 Anand, A. & Kaczkowski, P. J. NONINVASIVE MEASUREMENT OF LOCAL THERMAL DIFFUSIVITY USING BACKSCATTERED ULTRASOUND AND FOCUSED ULTRASOUND HEATING. 34, 1449-1464 (2008).
41 Wongsuwarn, S. et al. Giant thermophoresis of poly(N-isopropylacrylamide) microgel particles. Soft Matter 8, 5857-5863, doi:10.1039/C2SM25061F (2012).
42 Ramanujan, S. et al. Diffusion and Convection in Collagen Gels: Implications for Transport in the Tumor Interstitium. Biophysical Journal 83, 1650-1660, doi:10.1016/S0006-3495 (02) 73933-7 (2002).
43 Veys, I. et al. ICG fluorescence imaging as a new tool for optimization of pathological evaluation in breast cancer tumors after neoadjuvant chemotherapy. Plos One 13, e0197857, doi:10.1371/journal.pone.0197857 (2018).
44 Bourgeois, P. et al. Near-Infrared Fluorescence Imaging of Breast Cancer and Axillary Lymph Nodes After Intravenous Injection of Free Indocyanine Green. Frontiers in Oncology 11, doi:10.3389/fonc.2021.602906 (2021).
45 Intes, X. et al. In vivo continuous-wave optical breast imaging enhanced with Indocyanine Green. Medical Physics 30, 1039-1047, doi:https://doi.org/10.1118/1.1573791 (2003).
46 Corlu, A. et al. Three-dimensional in vivo fluorescence diffuse optical tomography of breast cancer in humans. Opt. Express 15, 6696-6716 (2007).
47 Ntziachristos, V., Yodh, A. G., Schnall, M. & Chance, B. Concurrent MRI and diffuse optical tomography of breast after indocyanine green enhancement. Proceedings of the National Academy of Sciences 97, 2767-2772, doi:10.1073/pnas. 040570597 (2000).
48 Schupper, A. J. et al. Fluorescence-Guided Surgery: A Review on Timing and Use in Brain Tumor Surgery. Frontiers in Neurology 12, doi:10.3389/fneur.2021.682151 (2021).
49 DSouza, A., Lin, H., Henderson, E., Samkoe, K. & Pogue, B. Review of fluorescence guided surgery systems: identification of key performance capabilities beyond indocyanine green imaging. J Biomed Opt 21, 080901 (2016).
50 Pogue, B., Rosenthal, E., Achilefu, S. & van Dam, G. Perspective review of what is needed for molecular-specific fluorescence-guided surgery. J Biomed Opt 23, 100601 (2018).
51 Pogue, B. & Rosenthal, E. Review of successful pathways for regulatory approvals in open-field fluorescence-guided surgery. J Biomed Opt 26, 030901 (2021).
52 Hagen, A. et al. Late-fluorescence mammography assesses tumor capillary permeability and differentiates malignant from benign lesions. Opt. Express 17, 17016-17033, doi:10.1364/OE.17.017016 (2009).
53 Liu, Y., Yao, T., Ren, L. & Yuan, B. Size effect of liposomes on centimeter-deep ultrasound-switchable fluorescence imaging and ultrasound-controlled release. Biomaterials Advances Submitted (2022).
54 Lankelma, J. et al. Doxorubicin Gradients in Human Breast Cancer. Clin Cancer Res 5, 1703-1707 (1999).
55 Lao, J. et al. Liposomal Doxorubicin in the Treatment of Breast Cancer Patients: A Review. Journal of Drug Delivery 2013, 456409, doi:10.1155/2013/456409 (2013).
56 Lee, S. C. et al. Accuracy of Contrast-Enhanced Ultrasound Compared With Magnetic Resonance Imaging in Assessing the Tumor Response After Neoadjuvant Chemotherapy for Breast Cancer. Journal of Ultrasound in Medicine 36, 901-911, doi:https://doi.org/10.7863/ultra.16.05060 (2017).

### EXAMPLE 5

### Deriving and Determining the Transportability Coefficient of an Agent

### A. Introduction

Drug delivery barriers in a tumor are a challenge and can significantly limit treatment efficacy and efficiency. Transport barriers are complicated because tumors usually have very complicated microenvironments, and many factors can contribute to transport barriers. For example, the proliferation of cancer cells can invade and occupy the extracellular space and degrade drug transportability. The elevated interstitial fluid (IFP) can stop natural convection transport mechanisms. The extracellular matrix (ECM) in some tumors may have limited porosity or permeability. The interstitial fluid in some tumors may be more viscous than in normal tissues. Unfortunately, it is almost impossible to quantify these factors individually.

Because of the complexity of transport barriers, there is no simple way to define the transportability of a drug or delivery vehicle in a tumor, and no imaging technology is available to quantify it. Tumor transport barriers are usually caused by different mechanisms simultaneously, and solving one of them may not overcome others. To better understand transport barriers for different cancers and further overcome them by developing new drugs, delivery vehicles, or technologies accelerating transport, defining a widely applicable parameter to quantify tumor transportability is highly desirable.

In this Example, this disclosure presents the mathematical and experimental derivation of the transportability coefficient (M), a “diffusion-like” coefficient, of a fluorophore-labeled agent. In fluorescence recovery after ultrasound squeezing (FRAUS), a focused ultrasound beam squeezes tissue in its focal volume and “pumps” fluorophore-labeled drug-delivery agents out of the focal volume via the SIF-TUM mechanism. After squeezing the tissue, fluorophore-labeled agents flow back into the original focal volume from the surrounding areas because of tissue recovery. This procedure can be monitored by the recovery of the USF signal. The recovered USF signal is used to calculate the transportability coefficient of the agent. M is used to represent the transportability of the agent in the focal volume. M includes all the major transport barrier factors and can be applied to different cancers as a widely applicable parameter. If multiple points or an image is needed, scanning the ultrasound focus 2- or 3-dimensionally and repeating the above procedures can be conducted.

### B. Mathematically Deriving the Transportability Coefficient M

Inserting the equations 1 and 2 (2a and/or 2b) into equation 4 (4a and/or 4b) listed in Table 1 in Example 1, parameter M can be derived as M=R_{f}KH=R_{f} (k/μ)H, which represents the transportability of an agent in tissue. M depends on four factors: (1) tissue permeability k, which is related to tissue porosity; (2) tissue fluid viscosity (μ); (3) the retardation factor between interstitial fluid and the agent R_{f}, which is related to the size difference between tissue pores and nanoagent, and also the surface charge effect between the agent and tissue can be incorporated here; and (4) tissue apparent modulus H, which is related to tissue elasticity. These factors are unmeasurable individual factors.

### C. Physical Understanding of the Transportability Coefficient M

The unit of M is meter²/s, which is a unit describing a diffusion process. Therefore, it can also be called “agent recovery diffusion coefficient.” It represents the capability of agents to flow back into the ultrasound focal volume after ultrasound squeezing via a “diffusion-like” behavior, rather than being caused by a concentration gradient.

### D. Determination of the Transportability Coefficient M from Experimental Data

The time derivative of the measured USF signal at the central location of the ultrasound focal volume (i.e., r=0) is the backflow velocity and denoted as vₜ=dS_{USF}/dt|_{r=0,t>t}_{₀}. Here, to is the time when SIF-TUM ultrasound exposure ends. To eliminate the effect of any unknown experimental parameters, such as optical-, ultrasound-, tissue-, and agent-related parameters, v₀=dS_{USF}/dt|_{r=0,t=t}_{₀} is used as a normalized factor, which is the initial backflow velocity right after SIF-TUM squeezing. Once vₜ/v₀ is obtained, it can be fitted to the numerical solution of equations 2 and 4 in Table 1 to extract M.

This method has the following advantages: (1) M is positively correlated with transportability; (2) vₜ/v₀ is a ratio, eliminating the effects from unknown experimental factors; and (3) M is the only parameter that can affect the shape of vₜ/v₀ in the fitting. Thus, not intending to be bound by theory, the fitting is likely a single solution problem. All these advantages make the method robust.

### E. Conclusion

The recovered USF signal of FRAUS can be used to calculate the transportability coefficient, a “diffusion-like” coefficient (M), of fluorophore-labeled drug-delivery agents. M can be determined from experimental data. The use of M is helpful in understanding the tumor transportability of agents

Many different arrangements of the various components and/or steps depicted and/or described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A method of selectively transporting an agent within a porous material, the method comprising:
disposing a population of agents in the porous material;
applying a focused ultrasound beam to a first target region of the porous material to compress at least a portion of the first target region; and
removing the focused ultrasound beam from the first target region, wherein the focused ultrasound beam has a duty cycle greater than 5%.

2. The method of claim 1, wherein applying the focused ultrasound beam creates a first order ultrasound oscillation wave having a pressure gradient (∇P₁) in one, two, or three dimensions of 0.1 MPa/mm to 10 MPa/mm.

3. The method of claim 1, wherein:
the focal volume of the focused ultrasound beam has a peak pressure of 1 kPa to 10 MPa; and/or
the focused ultrasound beam has a frequency of 1-30 MHz.

4. The method of claim 1, wherein the porous material comprises solid matrix material and fluid material.

5. The method of claim 4, wherein the porous material comprises a biological compartment.

6. The method of claim 5, wherein the solid matrix material comprises cells or tissue.

7. The method of claim 5, wherein the fluid material comprises interstitial fluid.

8. The method of claim 1, wherein applying the focused ultrasound beam to the first target region forces at least a portion of the population of agents out of the first target region.

9. The method of claim 1, further comprising:
applying the focused ultrasound beam to a second target region of the porous material to compress at least a portion of the second target region;
removing the focused ultrasound beam from the second target region,
wherein the second target region is different than the first target region;
applying the focused ultrasound beam to n additional target regions of the porous material to compress at least a portion of the n additional target regions; and
removing the focused ultrasound beam from the n additional target regions,
wherein n is an integer up to 10,000.

10. (canceled)

11. The method of claim 1, wherein exposure of the porous material to the focused ultrasound beam is associated with a mechanical index (MI) of less than 1.9.

12. The method of claim 1, wherein the agent changes size when exposed to the focused ultrasound beam and/or when exposed to a temperature change.

13. The method of claim 1, wherein the agent comprises a USF contrast agent.

14. The method of claim 1, wherein the population of agents has an average size, prior to applying the focused ultrasound beam, that is smaller than a pore size of the porous material.

15. The method of claim 1, wherein the population of agents has an average size, prior to applying the focused ultrasound beam, of less than 300 nm.

16. The method of claim 15, wherein the population of agents has an average size between 10 nm and 40 nm.

17. The method of claim 1, wherein applying the focused ultrasound beam to the first target region increases the temperature of the first target region by less than 5° C.

18. (canceled)

19. The method of claim 1, further comprising:
imaging the population of agents.

20. The method of claim 1, further comprising:
releasing a payload from the population of agents.

21. An ultrasound system comprising:
one or more ultrasound sources;
a control system;
optionally, a fluorophore excitation source; and
optionally, an image recording device,
wherein the control system carries out one or more steps of the method of claim 1.

22. A method of characterizing a biological tissue, the method comprising:
disposing a population of ultrasound-switchable fluorophores in a first region of the biological tissue;
applying a focused ultrasound beam to the first region to switch at least one fluorophore of the population from an off state to an on state;
applying a beam of electromagnetic radiation to the first region to excite at least one fluorophore of the population in the on state;
removing the focused ultrasound beam from the first region; and
detecting a dynamic ultrasound fluorescence (USF) signal emitted by the population of fluorophores during a recovery period after removal of the focused ultrasound beam from the first region;
measuring at least one of the following:
a recovery time constant (τ) for the first region;
a transportability coefficient (M) for the first region.

23-37. (canceled)
